(19) United States
(12) Patent Application Publication
(10) Pub. No.: US 2010/0076005 A1
(43) Pub. Date: Mar. 25, 2010

(54) ISOCARBOSTYRIL ALKALOID DERIVATIVES HAVING ANTI-PROLIFERATIVE AND ANTI-MIGRATORY ACTIVITIES

(75) Inventors: Laurent Ingrassia, Braine L'Alleud (BE); Lise Wlodarczak, Faimes (BE); Stephanie Thomas, Boneffe (BE); Eric Van Quaquebeke, Bruxelles (BE); Laurent Van den Hove, Forest (BE); Robert Kiss, St-Pieters-Leeuw (BE); Francis Darro, Waterloo (BE)

Correspondence Address: KNOBBE MARTENS OLSON & BEAR LLP, 2040 MAIN STREET, FOURTEENTH FLOOR, IRVINE, CA, 92614, US

(73) Assignee: Unibioscreen SA, Brussels (BE)

(21) Appl. No.: 12/445,405

(22) PCT Filed: Oct. 12, 2007
(86) PCT No.: PCT/EP2007/060906

§ 371 Date: Apr. 13, 2009

(30) Foreign Application Priority Data
Oct. 13, 2006 (EP) PCT/EP2006/009922

(51) Int. Cl. A61K 31/4355; C07D 491/02; A61P 35/04

(52) U.S. Cl. 514/280; 546/65

(57) ABSTRACT

Isocarbostyril alkaloid derivatives having an anti-proliferative and anti-migratory activities are disclosed. In particular, compounds of formula (I) or (II), as well as stereoisomers, tautomers, racemates, prodrugs, metabolites thereof, pharmaceutically acceptable salt and/or solvate are encompassed which are useful in the treatment and prophylaxis of cancer. Methods of preparation are also disclosed.

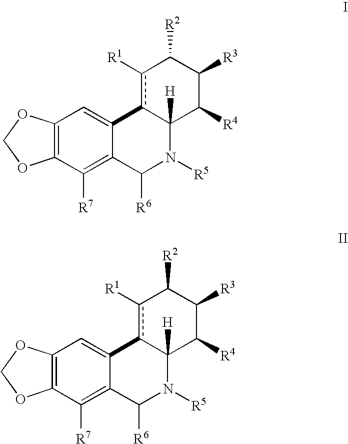

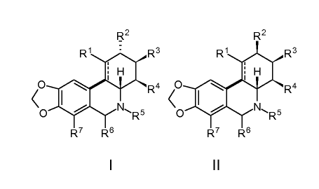

### FIELD OF THE INVENTION

The present invention relates to novel isocarbostyril alkaloid derivatives having a pharmacological activity, in particular an anti-proliferative and anti-migratory activities. The present invention also relates to a method for the preparation of said compounds. The invention further relates to a pharmaceutical composition comprising an effective amount of said compounds and to the use of said compounds as a medicament and/or for the (preparation of a medicament for the) treatment of diseases associated with cell proliferation, and even in particular in the treatment of cancer.

### BACKGROUND OF THE INVENTION

Plants from the Amaryllidaceae family have been known since a long time for their medicinal and toxic properties as well. For instance, the oil from the daffodil Narcissus poeticus L. was already employed in the Ancient Greece as a remedy for the treatment of cancer-related diseases (Hartwell J L, Lloydia. 30: 379, 1967). Among numerous compounds isolated from Amaryllidaceae, the hydroxylated phenantridones, also referred to as the Amaryllidaceae isocarbostyrils, have been under intense scrutiny, mainly because of their promising anti-tumor activity. Lycorine was the first member of this group of compounds to be isolated (Gorter K, Bull. Jard. Bot. 1: 352-8, 1920), and was known to possess antitumor activity as soon as 1958 (Fitzgerald R et al., Natl. Cancer Inst. 20: 763, 1958), well before the identification of other structurally related congeners. Lycoricidine (7-deoxynarciclasine) and narciclasine (lycoricidinol) were isolated from the bulbs of Lycoris radiate in 1967 (Ceriotti G, Nature. 213(76): 595-6, 1967; Okamoto T et al. Chem. Pharm. Bull. (Tokyo): 16(9): 1860-4, 1968). Sixteen years later, in 1984, pancratistatin was purified from the bulbs of Pancratium littorale (Pettit G R et al., J. Nat. Prod. 47(6): 1018-20, 1984; Pettit G R et al., J. Nat. Prod. 49(6): 995-1002, 1986).

Most chemotherapeutic agents currently used in the clinic for the treatment of cancer aim to take advantage of cell division itself and gain much of their selectivity from the fact that cancer cells divide more rapidly than their normal counterparts. In addition, the life duration of cancer cells is longer than that of normal cells in the tissue of which the cancer arose. A major problem with the above cited cytotoxic molecules reside in their lack of specificity: normal cells are themselves being injured. As a consequence, these drugs share in common an elevated toxicity. Therefore, there is nowadays a need for a new generation of molecules that target with more selectivity tumor cells.

In addition, cell migration plays an essential role in various biological processes, including cancer, throughout the life of most organisms. For instance, during development, defects in cell migration are responsible for severe congenital defects and disease. Cell migration is a highly complex process that is mediated by the actin-myosin cytoskeleton as well as microtubules and, in some cells, the intermediate filament system. Cell migration plays a key role in the development of cancer. Indeed, three main characteristics of cancer cells involve their ability to proliferate aggressively in an uncontrolled manner, their increased resistance to death-inducing stimuli and, third, their capacity to leave their primary tissue or organ and migrate towards new sites, leading to the development of metastasis. There is a need nowadays for drugs that target more or less specifically the migration process of cancer cells, therefore preventing them from conquering neighboring or even distant sites and organs, which is disastrous for the final outcome of the disease.

In summary, there remains an urgent need in the art for finding improved anti-cancer drugs, which overcome at least some of the above-mentioned drawbacks.

Consequently, it is a general object of the invention to provide improved anti-proliferative and/or cytotoxic drugs that target more or less specifically cancer cells as compared to normal cells. It is also an object of the present invention to provide improved anti-migratory drugs that aim to combat the migration of cancer cells and, consequently, the development of metastasis. In addition, more in particular, it is an object of the present invention to provide novel anti-cancer drugs and methods for synthesizing these. It is still another object of the invention to provide intermediate compounds as a result of the aforementioned synthesis methods, which have a pharmaceutical utility, e.g. in the treatment of proliferative diseases.

### SUMMARY OF THE INVENTION

In a first aspect the present invention relates to compounds of the formula I or II, stereoisomers, tautomers, racemates, prodrugs, metabolites thereof, or a pharmaceutically acceptable salt and/or solvate thereof,

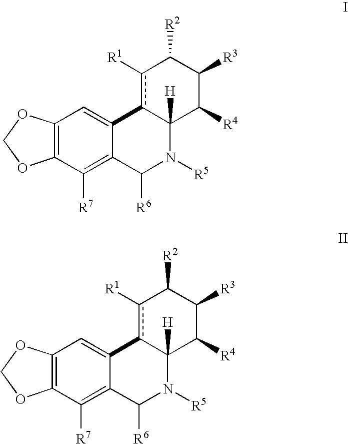

wherein
R¹ is selected from hydrogen or a group selected from hydroxyl, alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, Het¹carbonyloxy, Het²-carbonyloxy, cycloalkylcarbonyloxy, aralkyl, cycloalkyl, Het¹, Het², alkenyl, alkynyl, alkylamino, arylamino, alkylaminoalkyl, alkylcarbonyl, alkylcarbonylamino, amino, cycloalkyloxy, alkylthio, arylthio, formylamino, Het¹alkyl, Het²alkyl, Het¹oxy, Het²oxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, Het²amino, alkenylamino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyl, arylaminothiocarbonyloxy, alkenylcarbonyloxy, Het¹amino, Het²-amino, Het¹carbonyloxy, Het²-carbonyloxy, cycloalkylcarbonyloxy, alkoxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, aryloxy, or saccharyl, each group being optionally substituted with one or more substituents independently selected from halogen, thiol, alkyl, haloalkyl, alkoxy, acyl, carboxyamino, acylamino, acetamido, haloalkoxy, alkylcarbonyl, aryl, aralkyl, Het¹, Het², cycloalkyl, carboxyl, oxo, cyano nitro, amino, aminoalkyl, alkylamino, alkylamido, arylamino, Het¹amino, Het²-amino, alkenylamino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, hydroxyalkyl, or phosphatyl,
R³ is a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²-amino, Het¹carbonyloxy, Het²-carbonyloxy, cycloalkylcarbonyloxy, alkoxy, aryloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano nitro, aminoalkyl, alkylamino, arylamino, Het¹amino, Het²-amino, alkenylamino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
R⁴ is a group selected from hydroxyl, alkylcarbonyloxy, alkoxy, aryloxy, sulfato, arylalkyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²-amino, Het¹carbonyloxy, Het²-carbonyloxy, cycloalkylcarbonyloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, alkyloxycarbonylalkyl, carboxyalkyl, alkylaminoalkyl, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, Het²-amino, alkenylamino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
or wherein R³ and R⁴ together with the carbon atoms to which they are attached form a dioxolane ring, said ring being optionally substituted with one or two substituents selected from alkyl or aryl, R⁵ is hydrogen or a group selected from alkyl, alkyloxycarbonylalkyl, aryloxycarbonylalkyl, aryl, aralkyl, alkenyl, alkynyl, alkylaminoalkyl, alkylcarbonyl, arylcarbonyl, cycloalkyl, Het¹alkyl, Het²alkyl, alkyloxycarbonyl, aminocarbonyl, haloalkylcarbonylaminocarbonyl, alkylcarbonylaminocarbonyl, or cycloalkylalkyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, Het²-amino, alkenylamino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
R⁶ is hydrogen, oxo or thioxo,
R⁷ is hydrogen, hydroxyl or a group selected from alkoxy, alkenyloxy, alkynyloxy, aralkyloxy, alkylcarbonyloxy, alkenylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, Het¹carbonyloxy, Het²-carbonyloxy, aryloxy, sulfato, Het¹oxy, Het²oxy, alkylaminoalkyloxy, cycloalkyloxy, cycloalkylalkyloxy, Het¹alkyloxy, Het²alkyloxy, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, —O—R⁸—O—R⁹, or saccharyl wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, acyl, carboxyamino, acylamino, acetamido, haloalkoxy, alkylcarbonyl, arylcarbonyl, aryl, aralkyl, Het¹, Het², cycloalkyl, carboxyl, oxo, cyano, nitro, amino, aminoalkyl, alkylamino, arylamino, alkylamido, Het¹amino, Het²-amino, or phosphatyl,
and wherein the dotted line represents an optional double bond,
with the proviso that:
when R¹ is hydrogen, R² and R⁴ are hydroxyl, R⁵ is hydrogen or methyl, and R⁶ is an oxo, then R⁷ is not hydroxyl when R³ is hydroxyl,
when R¹ is hydrogen, R² and R⁴ are hydroxyl, R⁵ is hydrogen or methyl, R⁶ is an oxo, and the dotted line represents a double bound, then R⁷ is not hydroxyl or methoxy when R³ is hydroxyl or methoxy,
when R¹, R², R⁴ are acetoxy, R⁵ is hydrogen, R⁶ is an oxo, and R⁷ is hydroxyl, methoxy, or acetoxy, then R³ is not hydroxyl or acetoxy,
when R¹ is hydrogen, R³ and R⁴ together with the carbon atoms to which they are attached form 2,2-dimethyl-1,3-dioxolane, R⁵ is hydrogen, 4-methoxybenzyl, or methyl, and R⁶ is an oxo, then R⁷ is not hydrogen or hydroxyl when R² is hydroxyl, methoxy, ethoxy, benzyloxy or acetoxy, and R⁷ is not acetoxy when R² is acetoxy, and R⁷ is not methoxy when R² is hydroxyl, methoxy, or acetoxy,
when R², R³, and R⁴ are acetoxy, R⁵ is hydrogen, R⁶ is an oxo, and R¹ is hydrogen, benzoyloxy, or acetoxy, then R⁷ is not hydrogen, hydroxyl, acetoxy, or methoxy,
when R², R³, and R⁴ are hydroxyl, R⁵ is hydrogen or 4-methoxybenzyl, R⁶ is an oxo, and R⁷ is hydrogen, methoxy or hydroxyl, then R¹ is not tetrahydropyranyloxy, phenyloxycarbonyl, benzoyloxy, benzyloxy, or 1-O-3-hydroxybutyryl,
when R¹ is hydrogen, R³ and R⁴ are acetoxy, R⁵ is hydrogen, R⁶ is an oxo, and R⁷ is methoxy or acetoxy, then R² is not benzoyloxy or acetoxy,
when R¹ is hydrogen, R³ and R⁴ are methoxy, R⁵ is methyl, R⁶ is an oxo, and R⁷ is methoxy, then R² is not methoxy,
when R², R³, and R⁴ are acetoxy, R⁵ is hydrogen, R⁶ is an oxo, and R⁷ is acetoxy, then R¹ is not hydrogen,
when R², R³, and R⁴ are hydroxyl, R⁵ is hydrogen, R⁶ is an oxo, and R⁷ is hydroxyl or methoxy, then R¹ is not benzoyloxy,
when R¹ is hydroxyl, R³ and R⁴ together with the carbon atoms to which they are attached form 2,2-dimethyl-1,3-dioxolane, R⁵ is hydrogen or 4-methoxybenzyl, and R⁶ is an oxo, then R⁷ is not hydrogen hydroxyl, methoxy, or acetoxy when R² is hydroxyl or acetoxy,
when R¹ is hydroxyl or hydrogen, R², R³, and R⁴ are hydroxyl, R⁵ is hydrogen, R⁶ is an oxo, then R⁷ is not hydrogen, hydroxyl, methoxy, or benzyloxy,
when R¹ is hydrogen, R², R³, and R⁴ are benzoyloxy, R⁵ is hydrogen, benzoyl, or benzoyloxy, R⁶ is an oxo, then R⁷ is not hydrogen or methoxy,
when R¹ is hydrogen, R² and R⁴ and R⁵ are benzoyloxy, R⁶ is an oxo, and R⁷ is methoxy, then R³ is not methoxy,
when R¹, R², R³, and R⁴ are benzoyloxy, R⁵ is hydrogen or benzoyl, and R⁶ is an oxo, then R⁷ is not hydrogen,
when R¹ is acetyloxy, R², R³, and R⁴ are benzoyloxy, R⁵ is hydrogen, R⁶ is an oxo, then R⁷ is not hydrogen,
when R¹, R², R³ and R⁴ are hydroxyl or acetyloxy, R⁵ is hydrogen, R⁶ is oxo, R⁷ is not phosphatyl,
when R¹ is hydrogen, R², R³, and R⁴ are acetoxy, R⁵ is hydrogen, R⁶ is an oxo, then R⁷ is not hydrogen, and
when R⁶ is hydrogen, R⁷ is not hydrogen.

The invention further provides a pharmaceutical composition comprising a pharmaceutically acceptable excipient and a therapeutically effective amount of at least one compound according to the invention.

The invention further provides for the use of a compound according to the invention for the preparation of a medicament for the prevention and/or treatment of cancer, and/or for preventing, treating and/or alleviating complications and/or symptoms and/or inflammatory responses associated therewith.

The invention also provides a method for treating cancer using a medicament comprising at least one compound of formula I or II as described above as an active ingredient, such that the cancer is treated.

The present invention further provides kits for use in treating cancer and related disorders in an individual in need thereof comprising a therapeutically effective amount of the pharmaceutical composition comprising at least one compound of formula I or II, optionally, in combination with a pharmaceutically acceptable carrier.

Other aspects, embodiments, uses and advantages of the invention will become clear from the further description below.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Preferably, the present invention provides compounds of formula I or II, or a stereoisomer, tautomer, racemate, metabolite, pro- or predrug, salt, hydrate, or solvate thereof, wherein

R¹ is hydrogen or a group selected from hydroxyl, C₁₋₆alkyl, C₅₋₈aryl, C₁₋₆alkoxy, C₅₋₈aryloxy, C₁₋₆alkylaminocarbonyloxy, C₅₋₈arylaminocarbonyloxy, C₁₋₆alkylaminothiocarbonyloxy, C₅₋₈arylaminothiocarbonyloxy, C₁₋₆alkylcarbonyloxy, C₅₋₈arylcarbonyloxy, Het¹carbonyloxy, Het²-carbonyloxy, C₃₋₈cycloalkylcarbonyloxy, C₅₋₈arylC₁₋₆alkyl, C₃₋₈cycloalkyl, Het¹, Het², C₂₋₆alkenyl, C₂₋₆alkynyl, C₁₋₆alkylamino, C₅₋₈arylamino, C₁₋₆alkylaminoC₁₋₆alkyl, C₁₋₆alkylcarbonyl, C₁₋₆alkylcarbonylamino, amino, C₃₋₈cycloalkyloxy, C₁₋₆alkylthio, C₅₋₈arylthio formylamino, Het¹C₁₋₆alkyl, Het²C₁₋₆alkyl, Het¹oxy, Het²oxy, or saccharyl, each group being optionally substituted with one or more substituents independently selected from halogen, C₁₋₆alkyl, haloC₁₋₆alkyl, C₁₋₆alkoxy, haloC₁₋₆alkoxy, C₁₋₆alkylcarbonyl, C₅₋₈aryl, C₅₋₈arylC₁₋₆alkyl, Het¹, Het², C₃₋₈cycloalkyl, carboxyl, oxo, cyano, nitro, amino, aminoC₁₋₆alkyl, C₁₋₆alkylamino, C₂₋₆alkenylamino, C₅₋₈arylamino, Het¹amina, Het²amino, C₁₋₆alkylaminosulfonyl, C₁₋₆alkylcarbonylamino, C₁₋₆alkyloxyaminoC₂₋₆alkenyl, C₁₋₆alkoxycarbonyl, C₁₋₆alkylsulfonyl, C₁₋₆alkylsulfonylamino, C₁₋₆alkylthio, C₅₋₈arylcarbonyl, C₅₋₈arylcarbonylamino, C₅₋₈aryloxy, haloC₅₋₈aryl, Het¹C₁₋₆alkyl, Het²C₁₋₆alkyl, or hydroxyC₁₋₆alkyl,
R² is oxo or a group selected from hydroxyl, C₁₋₆alkylcarbonyloxy, C₅₋₈arylcarbonyloxy, amino, C₁₋₆alkylamino, C₅₋₈arylamino, C₁₋₆alkylaminocarbonyloxy, C₅₋₈arylaminocarbonyloxy, C₁₋₆alkylaminothiocarbonyloxy, C₅₋₈arylaminothiocarbonyloxy, Het¹amino, Het²-amino, Het¹carbonyloxy, Het²-carbonyloxy, C₃₋₈cycloalkylcarbonyloxy, C₂₋₆alkenylcarbonyloxy, C₁₋₆alkoxy, C₅₋₈aryloxy, C₂₋₆alkenyloxy, C₂₋₆alkynyloxy, C₁₋₆alkylaminoC₁₋₆alkyl, C₁₋₆alkylcarbonyl, C₁₋₆alkylcarbonylamino, C₃₋₈cycloalkyloxy, C₁₋₆alkylthio, formylamino, Het¹oxy, Het²oxy, Het¹C₁₋₆alkoxy, Het²C₁₋₆alkoxy, C₅₋₈aryloxy, or saccharyl, each group being optionally substituted with one or more substituents independently selected from halogen, thiol, C₁₋₆alkyl, haloC₁₋₆alkyl, C₁₋₆alkoxy, acyl, carboxyamino, acylamino, acetamido, haloC₁₋₆alkoxy, C₁₋₆alkylcarbonyl, C₅₋₈aryl, C₅₋₈arylC₁₋₆alkyl, Het¹, Het², C₃₋₈cycloalkyl, carboxyl, oxo, cyano nitro, amino, aminoC₁₋₆alkyl, C₁₋₆alkylamino, C₂₋₆alkenylamino, C₁₋₆alkylamido, C₅₋₈arylamino, Het¹amino, Het²-amino, C₁₋₆alkylaminosulfonyl, C₁₋₆alkylcarbonylamino, C₁₋₆alkoxyaminoC₂₋₆alkenyl, C₁₋₆alkoxycarbonyl, C₁₋₆alkylsulfonyl, C₁₋₆alkylsulfonylamino, C₁₋₆alkylthio, C₅₋₈arylcarbonyl, C₅₋₈arylcarbonylamino, C₅₋₈aryloxy, haloC₅₋₈aryl, Het¹C₁₋₆alkyl, Het²C₁₋₆alkyl, hydroxyC₁₋₆alkyl, or phosphatyl,
R³ is a group selected from hydroxyl, C₁₋₆alkylcarbonyloxy, C₅₋₈arylcarbonyloxy, amino, C₁₋₆alkylamino, C₅₋₈arylamino, C₁₋₆alkylaminocarbonyloxy, C₅₋₈arylaminocarbonyloxy, C₁₋₆alkylaminothiocarbonyloxy, C₅₋₈arylaminothiocarbonyloxy, Het¹amino, Het²-amino, Het¹carbonyloxy, Het²-carbonyloxy, C₃₋₈cycloalkylcarbonyloxy, C₁₋₆alkoxy, C₅₋₈aryloxy, C₂₋₆alkenyloxy, C₂₋₆alkynyloxy, C₁₋₆alkylaminoC₁₋₆alkyl, C₁₋₆alkylcarbonyl, C₁₋₆alkylcarbonylamino, C₃₋₈cycloalkyloxy, C₁₋₆alkylthio, formylamino, Het¹oxy, Het²oxy, Het¹C₁₋₆alkoxy, Het²C₁₋₆alkoxy, or saccharyl, each group being optionally substituted with one or more substituents independently selected from halogen, C₁₋₆alkyl, haloC₁₋₆alkyl, C₁₋₆alkoxy, haloC₁₋₆alkoxy, C₁₋₆alkylcarbonyl, C₅₋₈aryl, C₅₋₈arylC₁₋₆alkyl, Het¹, Het², C₃₋₈cycloalkyl, carboxyl, oxo, cyano nitro, aminoC₁₋₆alkyl, C₁₋₆alkylamino, C₂₋₆alkenylamino, C₅₋₈arylamino, Het¹amino, Het²-amino, C₁₋₆alkylaminosulfonyl, C₁₋₆alkylcarbonylamino, C₁₋₆alkyloxyaminoC₂₋₆alkenyl, C₁₋₆alkoxycarbonyl, C₁₋₆alkylsulfonyl, C₁₋₆alkylsulfonylamino, C₁₋₆alkylthio, C₅₋₈arylcarbonyl, C₅₋₈arylcarbonylamino, C₅₋₈aryloxy, haloC₅₋₈aryl, Het¹C₁₋₆alkyl, Het²C₁₋₆alkyl, or hydroxyC₁₋₆alkyl,
R⁴ is a group selected from hydroxyl, C₁₋₆alkylcarbonyloxy, C₁₋₆alkoxy, C₅₋₈aryloxy, sulfato, arylC₁₋₆alkoxy, C₅₋₈arylcarbonyloxy, amino, C₁₋₆alkylamino, C₅₋₈arylamino, C₁₋₆alkylaminocarbonyloxy, C₅₋₈arylaminocarbonyloxy, C₁₋₆alkylaminothiocarbonyloxy, C₅₋₈arylaminothiocarbonyloxy, Het¹amino, Het²-amino, Het¹carbonyloxy, Het²-carbonyloxy, C₃₋₈cycloalkylcarbonyloxy, C₂₋₆alkenyloxy, C₂₋₆alkynyloxy, C₁₋₆alkylaminoC₁₋₆alkyl, C₁₋₆alkylcarbonyl, C₁₋₆alkylcarbonylamino, C₃₋₈cycloalkyloxy, C₁₋₆alkylthio, formylamino, Het¹oxy, Het²oxy, Het¹C₁₋₆alkoxy, Het²C₁₋₆alkoxy, C₁₋₆alkyloxycarbonylC₁₋₆alkyl, carboxyC₁₋₆alkyl, C₁₋₆alkylaminoC₁₋₆alkyl, or saccharyl, each group being optionally substituted with one or more substituents independently selected from halogen, C₁₋₆alkyl, haloC₁₋₆alkyl, C₁₋₆alkoxy, haloC₁₋₆alkoxy, C₁₋₆alkylcarbonyl, C₅₋₈aryl, C₅₋₈arylC₁₋₆alkyl, Het¹, Het², C₃₋₈cycloalkyl, carboxyl, oxo, cyano, nitro, amino, aminoC₁₋₆alkyl, C₁₋₆alkylamino, C₂₋₆alkenylamino, C₅₋₈arylamino, Het¹amino, Het²-amino, C₁₋₆alkylaminosulfonyl, C₁₋₆alkylcarbonylamino, C₁₋₆alkyloxyaminoC₂₋₆alkenyl, C₁₋₆alkoxycarbonyl, C₁₋₆alkylsulfonyl, C₁₋₆alkylsulfonylamino, C₁₋₆alkylthio, C₅₋₈arylcarbonyl, C₅₋₈arylcarbonylamino, C₅₋₈aryloxy, haloaryl, Het¹C₁₋₆alkyl, Het²C₁₋₆alkyl, or hydroxyC₁₋₆alkyl,
or wherein R³ and R⁴ together with the carbon atoms to which they are attached form a dioxolane ring, said ring being optionally substituted with one or two substituent selected from C₁₋₆alkyl or C₅₋₆aryl,
R⁵ is hydrogen or a group selected from C₁₋₆alkyl, C₁₋₆alkoxycarbonyC₁₋₆alkyl, C₅₋₈aryloxycarbonylC₁₋₆alkyl, C₅₋₈aryl, C₅₋₈arylC₁₋₆alkyl, C₂₋₆alkenyl, C₂₋₆alkynyl, C₁₋₆alkylaminoC₁₋₆alkyl, C₁₋₆alkylcarbonyl, C₃₋₈cycloalkyl, Het¹C₁₋₆alkyl, Het²C₁₋₆alkyl, C₁₋₆alkyloxycarbonyl, aminocarbonyl, haloC₁₋₆alkylcarbonylaminocarbonyl, C₁₋₆alkylcarbonylaminocarbonyl, or C₃₋₈cycloalkylC₁₋₆alkyl, each group being optionally substituted with one or more substituents independently selected from halogen, C₁₋₆alkyl, haloC₁₋₆alkyl, C₁₋₆alkoxy, haloC₁₋₆alkoxy, C₁₋₆alkylcarbonyl, C₅₋₈arylcarbonyl, C₅₋₈aryl, C₅₋₈arylC₁₋₆alkyl, Het¹, Het², C₃₋₈cycloalkyl, carboxyl, oxo, cyano, nitro, amino, aminoC₁₋₆alkyl, C₁₋₆alkylamino, C₂₋₆alkenylamino, C₅₋₈arylamino, Het¹amino, Het²-amino, C₁₋₆alkylaminosulfonyl, C₁₋₆alkylcarbonylamino, C₁₋₆alkyloxyaminoC₂₋₆alkenyl, C₁₋₆alkoxycarbonyl, C₁₋₆alkylsulfonyl, C₁₋₆alkylsulfonylamino, C₁₋₆alkylthio, C₅₋₈arylcarbonyl, C₅₋₈arylcarbonylamino, C₅₋₈aryloxy, haloC₅₋₈aryl, Het¹C₁₋₆alkyl, Het²C₁₋₆alkyl, or hydroxyC₁₋₆alkyl,
R⁶ is hydrogen, oxo, or thioxo,
R⁷ is hydrogen, hydroxyl or a group selected from C₁₋₆alkyloxy, C₂₋₆alkenyloxy, C₂₋₆alkynyloxy, C₅₋₈arylC₁₋₆alkyloxy, C₁₋₆alkylcarbonyloxy, C₅₋₈arylcarbonyloxy, C₅₋₈arylcarbonylC₁₋₆alkyloxy, carboxyC₁₋₆alkyloxy, C₁₋₆alkoxycarbonylC₁₋₆alkyloxy, C₅₋₈aryloxycarbonylC₁₋₆alkyloxy, Het¹carbonyloxy, Het²-carbonyloxy, C₅₋₈aryloxy, sulfato, Het¹oxy, Het²oxy, C₁₋₆alkylaminoC₁₋₆alkyloxy, C₃₋₈cycloalkyloxy, C₃₋₈cycloalkylC₁₋₆alkyloxy, Het¹C₁₋₆alkyloxy, Het²C₁₋₆alkyloxy, C₁₋₆alkylaminocarbonyloxy, C₅₋₈arylaminocarbonyloxy, C₁₋₆alkylaminothiocarbonyloxy, C₅₋₈arylaminothiocarbonyloxy, hydroxyC₁₋₆alkyloxy, amino, C₁₋₆alkylamino, C₂₋₆alkenylamino, C₁₋₆alkylcarbonylamino, C₅₋₈arylcarbonylamino, formylamino, C₅₋₈arylamino, Het¹amino, Het²amino, hydroxycarbonylC₁₋₆alkoxy, C₁₋₆alkylsulfonylamino, C₃₋₈cycloalkylamino, phosphatyl, thiol, C₁₋₆alkylthio, C₅₋₈arylthio, C₁₋₆alkylsulfonyl, C₅₋₈arylsulfonyl, C₂₋₆alkenylcarbonyloxy, O—R⁸—O—R⁹, or saccharyl wherein is R⁸ is C₁₋₆alkylene or C₂₋₆alkenylene and R⁹ is selected from hydrogen, C₁₋₆alkyl, C₅₋₈aryl, Het¹, Het², or cycloalkyl; each group being optionally substituted with one or more substituents independently selected from halogen, thiol, C₁₋₆alkyl, haloC₁₋₆alkyl, C₁₋₆alkoxy, acyl, carboxyamino, acylamino, acetamido, haloC₁₋₆alkoxy, C₁₋₆alkylcarbonyl, C₅₋₈aryl, C₅₋₈arylC₁₋₆alkyl, Het¹, Het², C₃₋₈cycloalkyl, carboxyl, oxo, cyano, nitro, amino, aminoC₁₋₆alkyl, C₁₋₆alkylamino, C₅₋₈arylammino, C₁₋₆alkylamido, Het¹amino, Het²-amino, or phosphatyl, and wherein the dotted line represents an optional double bond, with the proviso as described above.

Unless a context dictates otherwise, asterisks are used herein to indicate the point at which a mono- or bivalent radical depicted is connected to the structure to which it relates and of which the radical forms part.

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 20 carbon atoms, more preferably from 1 to 10 carbon atoms, still more preferably 1 to 8 carbon atoms, in particular 1 to 6 carbon atoms, preferably 1 to 4 carbon atoms. Alkyl groups may be linear or branched, and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl and its isomers, octyl and its isomers, nonyl and its isomers; decyl and its isomers. C₁-C₆ alkyl includes all linear and branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers.

The term “optionally substituted alkyl” refers to an alkyl group optionally substituted with one or more substituents (for example 1 to 4 substituents, for example 1, 2, 3, or 4 substituents) at any available point of attachment. Non-limiting examples of such substituents include halogen, hydroxyl, carbonyl, nitro, amino, oximes, imines, azido, hydrazino, cyano, alkyl, aryl, Het², cycloalkyl, acyl, alkylamino, alkoxy, thiol, alkylthio, carboxylic acid, acylamino, alkyl esters, carbamates, thioamido, urea, sulphonamido, and the like.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyalkyl” refers to a —R^{a}—OH group wherein R^{a} is alkylene as defined herein. For example, “hydroxyalkyl” includes hydroxymethyl, 2-hydroxyethyl, 1-(hydroxymethyl)-2-methylpropyl, 1-(hydroxymethyl)-2-hydroxyethyl, 3,4-dihydroxybutyl, 1-methyl-1-hydroxyethyl, 2-hydroxy-2-methylpropyl, 1-hydroxypropyl, 2-hydroxypropyl, 3-hydroxypropyl, 2-hydroxybutyl, 3-hydroxybutyl, 4-hydroxybutyl, 2,3-dihydroxypropyl, 2,3-dihydroxybutyl, 2-(hydroxymethyl)-3-hydroxypropyl, and so forth. “Alkoxyalkyl” or “alkyloxyalkyl” refers to an alkyl group substituted with one to two of OR^{b}, wherein R^{b} is alkoxy as defined below. For example, “aralkyl” or “arylalkyl” refers to a substituted alkyl group as defined above wherein at least one of the alkyl substituents is an aryl as defined below, such as benzyl also abbreviated “bn” or “PhCH₂—” which is a methyl group substituted by a phenyl. For example, “Het²alkyl” refers to a substituted alkyl group as defined above, wherein at least one of the alkyl substituents is a heteroaryl (Het²) as defined below such as pyridinyl.

The term “cycloalkyl group” as used herein is a cyclic alkyl group, that is to say, a monovalent, saturated, or unsaturated hydrocarbyl group having 1, 2, or 3 cyclic structures. Cycloalkyl includes all saturated or partially saturated (containing 1 or 2 double bonds) hydrocarbon groups containing 1 to 3 rings, including monocyclic, bicyclic, or polycyclic alkyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. The further rings of multi-ring cycloalkyls may be either fused, bridged, and/or joined through one or more spiro atoms. Examples of cycloalkyl groups are: cyclopropyl, cyclobutyl, cyclopentyl, cyclohexyl, cycloheptyl, cyclooctyl, cyclononyl, and cyclodecyl with cyclopropyl being particularly preferred. An “optionally substituted cycloalkyl” refers to a cycloalkyl having optionally one or more substituents (for example 1 to 3 substituents, for example 1, 2, 3, or 4 substituents), selected from those defined above for substituted alkyl. When the suffix “ene” is used in conjunction with a cyclic group, this is intended to mean the cyclic group as defined herein having two single bonds as points of attachment to other groups.

Where alkyl groups as defined are divalent, i.e., with two single bonds for attachment to two other groups, they are termed “alkylene” groups. Where an alkylene or cycloalkylene biradical is present, connectivity to the molecular structure of which it forms part may be through a common carbon atom or different carbon atom, preferably a common carbon atom. To illustrate this applying the asterisk nomenclature of this invention, a C₃ alkylene group may be for example *—CH₂CH₂CH₂—*, *—CH(—CH₂CH₃)—* or *—CH₂CH(—CH₃)—*.Non-limiting examples of alkylene groups includes methylene, ethylene, methylmethylene, trimethylene, propylene, tetramethylene, ethylethylene, 1,2-dimethylethylene, pentamethylene, and hexamethylene. Similarly, where alkenyl groups as defined above and alkynyl groups as defined above, respectively, are divalent radicals having single bonds for attachment to two other groups, they are termed “alkenylene” and “alkynylene” respectively. The term “alkenyl” as used herein refers to an unsaturated hydrocarbyl group, which may be linear, branched, or cyclic, comprising one or more carbon-carbon double bonds. Alkenyl groups thus comprise two or more carbon atoms, preferably between 2 and 20 carbon atoms, more preferably between 2 and 10 carbon atoms, still more preferably between 2 and 8 carbon atoms, for example, between 2 and 6 carbon atoms. Examples of alkenyl groups are ethenyl, propenyl, 1-butenyl, 2-butenyl, 3-butenyl, 2-methylpropenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, and the like. An optionally substituted alkenyl refers to an alkenyl having optionally one or more substituents (for example 1, 2, or 3 substituents, or 1 to 2 substituents), selected from those defined above for substituted alkyl. Similarly to cycloalkyl groups, cycloalkenyl groups may be considered to be a subset of homocyclic rings discussed hereinafter.

The term “alkynyl” as used herein, similarly to alkenyl, refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Examples alkynyl groups are ethynyl, 2-propynyl, 2-butynyl, 3-butynyl, 2-pentynyl and its isomers, 2-hexynyl and its isomers, 2-heptynyl and its isomers, 2-octynyl and its isomers, and the like. An optionally substituted alkynyl refers to an alkynyl having optionally one or more substituents (for example 1 to 4 substituents, or 1 to 2 substituents), selected from those defined above for substituted alkyl. Similarly to cycloalkyl groups, cycloalkynyl groups may be considered to be a subset of homocyclic rings discussed hereinafter.

The term “Het¹” or “heterocyclyl” alone or in combination, is defined as a saturated or partially unsaturated monocyclic, bicyclic, or polycyclic heterocycle having preferably 3 to 12 ring members, more preferably 5 to 10 ring members, and more preferably 5 to 6 ring members, which contains one or more heteroatom ring members selected from nitrogen, oxygen, or sulfur, and which is optionally substituted on one or more carbon atoms by alkyl, alkoxy, halogen, hydroxyl, oxo, optionally mono- or disubstituted amino, nitro, cyano, haloalkyl, carboxyl, alkoxycarbonyl, cycloalkyl, optionally mono- or disubstituted aminocarbonyl, methylthio, methylsulfonyl, aryl, and a saturated or partially unsaturated monocyclic, bicyclic or tricyclic heterocycle having 3 to 12 ring members which contains one or more heteroatom ring members selected from nitrogen, oxygen, or sulfur, and whereby the optional substituents on any amino function are independently selected from alkyl, alkoxy, Het², Het²alkyl, Het²oxy, Het²oxyalkyl, aryl, aryloxy, aryloxyalkyl, aralkyl, alkyloxycarbonylamino, amino, and aminoalkyl whereby each of the amino groups may optionally be mono- or where possible di-substituted with alkyl.

Exemplary heterocyclic groups include aziridinyl, oxiranyl, thiiranyl, piperidinyl, azetidinyl, imidazolinyl, imidazolidinyl, pyrazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, and morpholinyl.

The term “aryl” as used herein refers to a polyunsaturated, aromatic hydrocarbyl group having a single ring (i.e. phenyl) or multiple aromatic rings fused together (e.g. naphthalene or anthracene), or linked covalently, typically containing 5 to 8 atoms; wherein at least one ring is aromatic. The aromatic ring may optionally include one to three additional rings (either cycloalkyl, Het¹, or Het²) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7-, or 8-azulenyl, 1- or 2-naphthyl, 1-, 2-, or 3-indenyl, 1-, 2-, or 9-anthryl, 1-2-, 3-, 4-, or 5-acenaphtylenyl, 3-, 4-, or 5-acenaphtenyl, 1-, 2-, 3-, 4-, or 10-phenanthryl, 1- or 2-pentalenyl, 1,2-, 3-, or 4-fluorenyl, 4- or 5-indanyl, 5-, 6-, 7-, or 8-tetrahydronaphthyl, 1,2,3,4-tetrahydronaphthyl, 1,4-dihydronaphthyl, dibenzo[a,d]cylcoheptenyl, 1-, 2-, 3-, 4-, or 5-pyrenyl.

The aryl ring can optionally be substituted by one or more substituents. An “optionally substituted aryl” refers to an aryl having optionally one or more substituents (for example 1 to 5 substituents, for example 1, 2, 3, or 4) at any available point of attachment. Non-limiting examples of such substituents are selected from halogen, hydroxyl, oxo, nitro, amino, hydrazine, aminocarbonyl, azido, cyano, alkyl, cycloalkyl, alkenyl, alkynyl, cycloalkylalkyl, alkylamino, alkoxy, —SO₂—NH₂, aryl, heteroaryl, arylalkyl, haloalkyl, haloalkoxy, alkyloxycarbonyl, alkylaminocarbonyl, heteroarylalkyl, alkylsulfonamido, heterocyclyl, alkylcarbonylaminoalkyl, aryloxy, alkylcarbonyl, acyl, arylcarbonyl, aminocarbonyl, alkylsulfoxide, —SO₂R^{c}, alkylthio, carboxyl, and the like, wherein R^{c} is alkyl or cycloalkyl.

The term “arylene” as used herein is intended to include divalent carbocyclic aromatic ring systems such as phenylene, biphenylylene, naphthylene, anthracenylene, phenanthrenylene, fluorenylene, indenylene, pentalenylene, azulenylene, and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene, and the like.

Where a carbon atom in an aryl group is replaced with a heteroatom, the resultant ring is referred to herein as a heteroaryl ring or Het².

The term “Het²” or “heteroaryl” as a group or part of a group is defined as an aromatic monocyclic, bicyclic, or tricyclic heterocycle having preferably 3 to 12 ring members, more preferably 5 to 10 ring members, and more preferably 5 to 6 ring members, which contains one or more heteroatom ring members selected from nitrogen, oxygen, or sulfur, and which is optionally substituted on one or more carbon atoms by alkyl, alkoxy, halogen, hydroxyl, optionally mono- or disubstituted amino, nitro, cyano, haloalkyl, carboxyl, alkoxycarbonyl, cycloalkyl, optionally mono- or disubstituted aminocarbonyl, methylthio, methylsulfonyl, aryl, Het¹, and an aromatic monocyclic, bicyclic, or tricyclic heterocycle having 3 to 12 ring members; whereby the optional substituents on any amino function are independently selected from alkyl, alkoxy, Het¹, Het¹alkyl, Het¹oxy, Het¹oxyalkyl, aryl, aryloxy, aryloxyalkyl, aralkyl, alkyloxycarbonylamino, amino, and aminoalkyl whereby each of the amino groups may optionally be mono- or where possible di-substituted with alkyl. Non-limiting examples of a Het² include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 1,3-benzodioxolyl, quinolinyl, isoquinolinyl, cinnolinyl, quinazolinyl, quinoxalinyl.

In particular, non-limiting examples of heteroaryl can be 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, 1,2,3-triazol-1-, -2-, -4-, or -5-yl, 1,2,4-triazol-1-, -3-, -4-, or -5-yl, 1,2,3-oxadiazol-4- or -5-yl, 1,2,4-oxadiazol-3- or -5-yl, 1,2,5-oxadiazolyl, 1,3,4-oxadiazolyl, 1,2,3-thiadiazol-4- or -5-yl, 1,2,4-thiadiazol-3- or -5-yl, 1,2,5-thiadiazol-3- or -4-yl, 1,3,4-thiadiazolyl, 1- or 5-tetrazolyl, 2-, 3-, or 4-pyridyl, 3- or 4-pyridazinyl, 2-, 4-, 5-, or 6-pyrimidinyl, 2-, 3-, 4-, 5-, or 6-2H-thiopyranyl, 2-, 3-, or 4-4H-thiopyranyl, 2-, 3-, 4-, 5-, 6-, or 7-benzofuryl, 1-, 3-, 4-, or 5-isobenzofuryl, 2-, 3-, 4-, 5-, 6-, or 7-benzothienyl, 1-, 3-, 4-, or 5-isobenzothienyl, 1-, 2-, 3-, 4-, 5-, 6-, or 7-indolyl, 2- or 3-pyrazinyl, 1,4-oxazin-2- or -3-yl, 1,4-dioxin-2- or -3-yl, 1,4-thiazin-2- or -3-yl, 1,2,3-triazinyl, 1,2,4-triazinyl, 1,3,5-triazin-2-, -4, or -6-yl, thieno[2,3-b]furan-2-, -3-, -4-, or -5-yl, 1-, 2-, 4-, or 5-benzimidazolyl, 1-, 3-, 4-, 5-, 6-, or 7-benzopyrazolyl, 3-, 4-, 5-, 6-, or 7-benzisoxazolyl, 2-, 4-, 5-, 6-, or 7-benzoxazolyl, 3-, 4-, 5-, 6-, or 7-benzisothiazolyl, 2-, 4-, 5-, 6-, or 7-benzothiazolyl, 1-, 2-thianthrenyl, 3-, 4- or 5-isobenzofuranyl, 1-, 2-, 3-, 4-, or 9-xanthenyl, 1-, 2-, 3-, or 4-phenoxathiinyl, 2-, 3-pyrazinyl, 1-, 2-, 3-, 4-, 5-, 6-, 7-, or 8-indolizinyl, 2-, 3-, 4-, or 5-isoindolyl, 1-, 2-, 3-, 4-, 5-, 6-, or 7-indazolyl, 2-, 6-, 7-, or 8-purinyl, 4-, 5-, or 6-phthalazinyl, 2-, 3-, or 4-naphthyridinyl, 2-, 5-, or 6-quinoxalinyl, 2-, 4-, 5-, 6-, 7-, or 8-quinazolinyl, 1-, 2-, 3-, or 4-quinolizinyl, 2-, 3-, 4-, 5-, 6-, 7-, or 8-quinolinyl(quinolyl), 2-, 4-, 5-, 6-, 7-, or 8-quinazolyl, 1-, 3-, 4-, 5-, 6-, 7-, or 8-isoquinolinyl(isoquinolyl), 3-, 4-, 5-, 6-, 7-, or 8-cinnolinyl, 2-, 4-, 6-, or 7-pteridinyl, 1-, 2-, 3-, 4-, or 9-carbazolyl, 1-, 2-, 3-, 4-, 5-, 6-, 7-, 8-, or 9-carbolinyl, 1-, 2-, 3-, 4-, 5-, 6-, 7-, 8-, 9-, or 10-phenanthridinyl, 1-, 2-, 3-, or 4-acridinyl, 1-, 2-, 3-, 4-, 5-, 6-, 7-, 8-, or 9-perimidinyl, 2-, 3-, 4-, 5-, 6-, 7-, 8-, 9-, or 10-(1,7)phenanthrolinyl, 1- or 2-phenazinyl, 1-, 2-, 3-, 4-, or 10-phenothiazinyl, 3- or 4-furazanyl, 1-, 2-, 3-, 4-, or 10-phenoxazinyl, or additionally substituted derivatives thereof.

The term “halo” or “halogen” as a group or part of a group is generic for fluoro, chloro, bromo, or iodo.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl, and the like.

The term “alkoxy” as used refers to a radical having the Formula —OR^{d} wherein R^{d} is alkyl as defined herein. Preferably, alkoxy is C₁-C₁₀ alkoxy or C₁-C₆ alkoxy. Non-limiting examples of alkoxy include methoxy, ethoxy, n-propoxy, isopropoxy, n-butoxy, iso-butoxy, sec-butoxy, tert-butoxy, hexanoxy, and the like.

The term “aryloxy” as used herein denotes a group —O-aryl, wherein aryl is as defined above. The term “haloalkoxy” alone or in combination refers to a group of formula —O-alkyl wherein the alkyl group is substituted by 1, 2, or 3 halogen atoms. For example, “haloalkoxy” includes —OCF₃ and —OCHF₂.

The term “alkylcarbonyl” by itself or as part of another substituent refers to an alkanoyl group having 2 to 6 carbon atoms or a phenylalkanoyl group whose alkanoyl moiety has 1 to 4 carbon atoms, i.e. a carbonyl group linked to an alky radical more particularly, the group —COR¹, wherein R¹ is alkyl or substituted alkyl, as defined herein. Preferably, alkylcarbonyl is C₂-C₁₁ alkylcarbonyl or C₂-C₇ alkylcarbonyl. Said alkylcarbonyl can be exemplified by acetyl, propionyl, butyryl, valeryl, pivaloyl, phenylacetyl, phenylpropionyl, and phenylbutylyl.

The term “arylcarbonyl” or “aroyl” by itself or as part of another substituents refers to a group of formula —C(O)-aryl, wherein aryl is as defined above, such as for example benzoyl also abbreviated herein as “bz” or “Ph-CO—”.

The term “alkylcarbonyloxy” by itself or as part of another substituents refers to a group of formula —O—C(═O)R^{e} wherein R^{e} is alkyl as defined before.

The term “alkenylcarbonyloxy” by itself or as part of another substituents refers to a group of formula —O—C(═O)R^{ee} wherein R^{ee} is alkenyl as defined before.

The term “arylcarbonyloxy” by itself or as part of another substituents refers to a group of formula —O—C(═O)R^{f} wherein R^{f} is aryl as defined before.

The term “Het¹carbonyloxy” by itself or as part of another substituents refers to a group of formula —O—C(═O)R^{m} wherein R^{m} is Het¹ as defined before.

The term “Het²-carbonyloxy” by itself or as part of another substituents refers to a group of formula —O—C(═O)R^{e} wherein R^{e} is Het² as defined before.

The term “cycloalkylcarbonyloxy” by itself or as part of another substituents refers to a group of formula O—C(═O)R^{o} wherein R^{o} is cycloalkyl as defined before.

The term “arylalkyloxy” or “aralkoxy” means alkoxy as defined herein, wherein an alkyl hydrogen atom is replaced by an aryl as defined herein. Examples of aralkoxy radicals include 2-phenylethoxy, 2-phenyl-1-propoxy, and the like.

The term “alkylcarbonylalkyl” by itself or as part of another substituents refers to a group of formula —R^{p}—C(═O)—R^{p} wherein R^{p} is alkylene or alkylene substituted by alkyl, and R^{q} is alkyl or substituted alkyl as defined herein, wherein the substituents are the same as that described above for substituted alkyl and substituted aryl.

The term “arylcarbonylalkyl” by itself or as part of another substituents refers to a group of formula —R^{p}—C(═O)—R^{r} wherein R^{p} is alkylene or alkylene substituted by alkyl, and R^{r} is aryl or substituted aryl as defined herein, wherein the substituents are the same as that described above for substituted alkyl and substituted aryl.

The term “hydroxyl” refers to an —OH group.

The term “carboxy” or “carboxyl” refers to the group —CO₂H. Thus, a carboxyalkyl is an alkyl group as defined above having at least one substituent that is —CO₂H.

The term “oxo” as used herein refers to the group ═O, i.e., forms a carbonyl moiety with the carbon atom to which it is attached.

The term “thioxo” as used herein refers to the group ═S.

The term “nitro” refers to a group of formula —NO₂.

The term “cyano” refers to a group of formula —CN.

The term “amino” refers to the group NH₂.

The term “aminoalkyl” by itself or as part of another substituents refers to a group of formula —R^{g}—NR^{h}Rⁱ wherein R⁹ is alkylene or substituted alkylene, R^{h} is hydrogen, alkyl or substituted alkyl as defined herein, and Rⁱ is hydrogen or alkyl as defined herein.

The term “alkylamino” by itself or as part of another substituent refers to a group consisting of an amino groups attached to one or two independently selected and optionally substituted alkyl groups, cycloalkyl groups, arylalkyl, or cycloalkylalkyl groups i.e., alkyl amino refers to N(R^{j})(R^{k}) wherein R^{j} and R^{k} are each independently selected from hydrogen, cycloalkyl, arylalkyl, cycloalkylalkyl, or alkyl. Non-limiting examples of alkylamino groups include methylamino (NHCH₃), ethylamino (NHCH₂CH₃), n-propylamino, isopropylamino, n-butylamino, isobutylamino, sec-butylamino, tert-butylamino, n-hexylamino, and the like.

The term “alkylcarbonylamino” by itself or as part of another substituents refers to a group of formula —NH(C═O)R or —NR'(C═O)R, wherein R and R′ are each independently alkyl or substituted alkyl.

The term “alkylaminocarbonyl” by itself or as part of another substituents refers to a group of formula —(C═O)—NR^{d}R^{e} wherein R^{d} is hydrogen, alkyl, or substituted alkyl as defined herein, and R^{e} is alkyl or substituted alkyl, as defined herein.

The term “alkylaminocarbonyloxy” by itself or as part of another substituents refers to a group of formula —O—(C═O)—NR^{d}R^{e} wherein R^{d} is hydrogen, alkyl, or substituted alkyl as defined herein, and R^{e} is alkyl or substituted alkyl, as defined herein.

The term “arylaminocarbonyloxy” by itself or as part of another substituents refers to a group of formula —O—(C═O)—NR^{d}R^{e} wherein R^{d} is hydrogen, alkyl, or substituted alkyl as defined herein, and R^{e} is aryl or substituted aryl, as defined herein.

The term “arylamino” by itself or as part of another substituent refers to a group consisting of an amino group attached to one or two independently selected aryl groups as described before.

The term “Het¹amino” by itself or as part of another substituent refers to a group consisting of an amino group attached to one or two independently selected Het¹ groups as described before.

The term “Het²-amino” by itself or as part of another substituent refers to a group consisting of an amino group attached to one or two independently selected Het² groups as described before.

The term “alkylthio” by itself or as part of another substituent refers to a group consisting of a sulfur atom attached to one optionally substituted alkyl, cycloalkyl, arylalkyl, or cycloalkylalkyl. Non-limiting examples of alkylthio groups include methylthio(SCH₃), ethylthio(SCH₂CH₃), n-propylthio, isopropylthio, n-butylthio, isobutylthio, sec-butylthio, tert-butylthio, n-hexylthio, and the like.

The term “sulfato” alone or in combination refers to a group of formula —O—SO₂—OR^{p} also written as —SO₄R^{p}, wherein R^{p} is hydrogen or a counter ion such as Na, K Ca, Mg, or a quaternary amine such as quaternary ammonium or pyridinium.

The term “sulfo” alone or in combination refers to a group of formula —SO₂—OR also written as —SO₃R^{p}, wherein R^{p} is hydrogen or a counter ion such as Na, K Ca, Mg, or a quaternary amine such as quaternary ammonium or pyridinium.

The term “alkylsulfonylamino” alone or in combination refers to a group of Formula —NR^{v}—SO₂—R^{w} wherein R^{v} is hydrogen or alkyl as defined herein, and R^{w} is alkyl as defined herein.

The term “thiocarbonyl” by itself or as part of another substituents refers to a group —C(═S)—.

The term “alkylaminothiocarbonyloxy” by itself or as part of another substituents refers to a group of Formula —O—C(═S)—NR⁹R^{h} wherein R⁹ is selected from hydrogen or alkyl, and R^{h} is alkyl as defined herein.

The term “acetyl” by itself or as part of another substituents refers to a —C(═O)—CH₃ and the term “acetoxy” refers to the group —O—C(═O)—CH₃.

The term “Het¹alkyl” by itself or as part of another substituents refers to a group having one of the aforementioned Het¹ group attached to one of the aforementioned alkyl group, i.e., to a group —R^{s}-R^{t} wherein R^{s} is alkylene or alkylene substituted by alkyl group, and R^{t} is a Het¹ group.

The term “Het²alkyl” by itself or as part of another substituents refers to a group having one of the aforementioned Het² group attached to one of the aforementioned alkyl group, i.e., to a group R^{s}-R^{u} wherein R^{s} is alkylene or alkylene substituted by alkyl group, and R^{u} is a Het² group.

The term “alkylsilyloxy” by itself or as part of another substituents refers to a group of Formula —O—Si(R^{v})₂—R^{w} wherein each R^{v} is independently hydrogen or alkyl as defined herein, and R^{N} is alkyl as defined herein.

The term “phosphatyl” or “phosphate” by itself or as part of another substituents refers to a group of formula —O—P(═O)—(OR^{x})₂ wherein R^{x} is hydrogen, alkyl or aryl as defined herein.

The term “saccharyl” by itself or as part of another substituents refers to a mono-, di-, tri-, oligo-, or poly-saccharide moieties made up of n sugar subunits linked to each other by glycosidic bonds, which subunits, when n is grater that 1, may be the same or different in respect to the type of constituent sugar residues (e.g. homo- or heteropolymeric), the localization of axial and equatorial ring substituents, the number of carbon atoms and the ring carbon locations and orientations of hydroxyl groups. Said saccharide moiety can be optionally substituted by one or more substituents selected from alkyl, alkoxy, acyl, carboxy, carboxyamino, amino, acylamino, acetamido, alkylamino, alkylamido, halo, thio, nitro, oxo, and phosphatyl groups, wherein the substitution may be at one or more positions on the saccharyl. Moreover, the saccharyl may also be present as a deoxy saccharyl. The term “saccharyl” also encompasses the amino derivatives thereof (resulting from replacement of any hydroxyl in the saccharyl with an amino), the amido derivatives thereof (resulting from replacement of any hydroxyl in the saccharyl with an amido group), the thio derivatives thereof (resulting from replacement of any hydroxyl of the saccharide with a thiol group), the hydroxyl-protected derivatives thereof such as acetate or benzoyl derivatives thereof, or the carboxy derivatives thereof. The term “saccharyl” as used herein also encompasses stereoisomers, optical isomers, anomers, and epimers of said saccharyl moiety. Thus, a hexose moiety for example can be either an aldose or a ketose moiety, and can be of D- or L-configuration, can assume either an α or β conformation, and can be a dextro- or levo-rotatory with respect to plane-polarized light. The glycosyl terms such as “glucosyl” (also named “glucopyranosyl”) “rhamnosyl” etc refer to the residue formed by detaching the hydrogen from the anomeric hydroxy group of a saccharide. Non-limiting examples of saccharyl include glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, glucosaminyl, N-acetyl-glucosaminyl, octylglucopyranosyl, octylribofuranosyl, cyclohexylglucopyranosyl, cyclohexylxylofuranosyl, benzylg lucopyranosyl, benzylarabinofuranosyl, N-acetyl-lactosaminyl, acosaminyl, amicetosyl, amylosyl, apiosyl, arcanosyl, ascarylosyl, bacillosaminyl, boivinosyl, cellotriosyl, chacotriosyl, chalcosyl, cladinosyl, colitosyl, cymarosyl, daunosaminyl, desosaminyl, d-glycero-L-gulo-heptosyl, diginosyl, digitalosyl, dig itoxosyl, evalosyl, evernitrosyl, forosaminyl, fucosaminyl, garosaminyl, hamamelosyl, isolevoglucosenonyl, kanosaminyl, kansosaminyl, lactosaminyl, lactosediaminyl, fucitolyl, maltulosyl, mannosaminyl, melezitosyl, mycaminosyl, mycarosyl, mycinosyl, mycosaminyl, noviosyl, oleandrosyl, paratosyl, perosaminyl, planteosyl, pneumosaminyl, purpurosaminyl, quinovosaminyl, quinovosyl, rhamnitolyl, rhamnosaminyl, rhodinosyl, rhodosaminyl, sarmentosyl, solatriosyl, stachyosyl, streptosyl, umbelliferosyl, trehalosaminyl, 1,6-anhydro-D-glucopyranosyl, 1-hydroxy-α-D-allopyranosyl, 2,3:5,6-di-O-isopropylid ene-D-mannofuranosyl, 2-amino-2-deoxy-D-galactitolyl, 2-deoxyribosyl, 2-deoxyglucosyl, 5-amino-5-deoxy-D-glucopyranosyl, 6-deoxy-D-galactitolyl, 2-amino-2-deoxy glucosyl, 2-acetamido-2-deoxy-glucosyl, 2-amino-2-deoxy galactosyl, 2-acetamido-2-deoxy-galactosyl, 2-amino-2-deoxy mannosyl, 2-acetamido-2-deoxy-mannosyl, 2-acetamido-2-deoxy-4-O-β-D-galactosyl-D-glucosyl, 2-amino-2-deoxy-4-O-β-D-galactosyl-D-glucosyl, 6′-N-acetylglucosaminyllactosyl, 2-acetamido-2-deoxy-3-O-α-L-fucosyl-D-glucosyl, 6-O(2-acetamido-2-deoxy-β-D-glucosyl)-D-galactosyl, 2-acetamido-2-deoxy-3-O-β-D-galactosyl-D-glucosyl, 2′-acetamido-2′-deoxy-3-O-β-D-glucosyl-D-galactosyl, 3-fucosyl-D-lactosyl, 3-fucosyl-2-acetamido-2-deoxy-4-O-β-D-galactosyl-D-glucosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof.

In an embodiment, said saccharyl moiety, includes monosaccharide, L or D isomers thereof, α or β form thereof, pyranuronic or furanuronic form thereof, pyranose or furanose form thereof, combination thereof, deoxy derivatives thereof, carboxy derivatives thereof, hydroxyl protected derivatives thereof such as hydroxyl-protected acetate or benzoyl derivatives thereof, amino derivatives thereof optionally substituted, amido derivatives thereof, thio derivatives thereof, di-, tri-, oligo-, or polysaccharides thereof optionally substituted by one or more substituents.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

Where groups may be optionally substituted, such groups may be substituted with once or more, and preferably once, twice, or thrice. Substituents may be selected from, for example, the group comprising halo, hydroxyl, oxo, nitro, amido, carboxy, amino, cyano haloalkoxy, and haloalkyl.

As used herein the terms such as “alkyl, aryl, or cycloalkyl, each being optionally substituted with” or “alkyl, aryl, or cycloalkyl, optionally substituted with” refers to optionally substituted alkyl, optionally substituted aryl, and optionally substituted cycloalkyl.

Whenever used in the present invention the term “compounds of the invention” or a similar term is meant to include the compounds of general Formula I or II and any subgroup thereof. This term also refers to the compounds as depicted in Tables 3 to 8 and their derivatives, N-oxides, salts, solvates, hydrates, stereoisomeric forms, racemic mixtures, tautomeric forms, optical isomers, analogues, pro-drugs, esters, and metabolites, as well as their quaternized nitrogen analogues. The N-oxide forms of said compounds are meant to comprise compounds wherein one or several nitrogen atoms are oxidized to the so-called N-oxide.

As used in the specification and the appended claims, the singular forms “a”, “an,” and “the” include plural referents unless the context clearly dictates otherwise. By way of example, “a compound” means one compound or more than one compound.

The compounds of the invention may be in the form of pharmaceutically and/or veterinary acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid, and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I or II above.

The invention also generally covers all pharmaceutically acceptable predrugs and prodrugs of the compounds of Formula I or II, for which general reference is made to the prior art cited hereinbelow.

The term “pro-drug” as used herein means the pharmacologically acceptable derivatives such as esters, amides, and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793, and WO 99/33792 all incorporated herein by reference. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

The compounds of the invention contain one or more asymmetric carbon atoms that serve as a chiral center, which may lead to different optical forms (e.g. enantiomers or diastereoisomers). The invention comprises all such optical forms in all possible configurations, as well as mixtures thereof.

More generally, from the above, it will be clear to the skilled person that the compounds of the invention may exist in the form of different isomers and/or tautomers, including but not limited to geometrical isomers, conformational isomers, E/Z-isomers, stereochemical isomers (i.e. enantiomers and diastereoisomers) and isomers that correspond to the presence of the same substituents on different positions of the rings present in the compounds of the invention. All such possible isomers, tautomers, and mixtures thereof are included within the scope of the invention.

The compounds of Formula I or II may be prepared as described in the experimental section below using methods and chemistries with which those skilled in the art shall be familiar.

It will also be clear that when the desired compounds of the invention, and/or the starting materials, precursors, and/or intermediates used in the preparation thereof, contain functional groups that are sensitive to the reaction conditions used in the preparation of the compounds of the invention (i.e. that would undergo undesired reactions under those conditions if they were not suitably protected) can be protected during said reaction with one or more suitable protective group, which protective group can then be suitably removed after either completion of said reaction and/or as a later or final step in the preparation of the compounds of the invention. Protected forms of the inventive compounds are included within the scope of the present invention. Suitable protective groups, as well as methods and conditions for inserting them and removing them, will be clear to the skilled person and are generally described in the standard handbooks of organic chemistry, such as Greene and Wuts, “Protective groups in organic synthesis”, 3rd Edition, Wiley and Sons, 1999, which is incorporated herein by reference in its entirety. It will also be clear to the skilled person that compounds of the invention in which one or more functional groups have been protected with suitable functional groups can find use as intermediates in the production and/or synthesis of the compounds of the invention, and as such form a further aspect of the invention. The terms described above and others used in the specification are well understood to those in the art.

In an embodiment, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, 1,3-dioxolanyl, 1,4-oxathianyl, 1,4-dithianyl, 1,3,5-trioxanyl, and morpholin-4-yl.

In an embodiment, Het² as a group or part of a group is selected from pyrrolyl, furyl, thienyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl, 1(6H)-yl, 1,3-benzodioxolyl, quinolinyl, isoquinolinyl, cinnolinyl, quinazolinyl, or quinoxalinyl. According to a preferred embodiment, the compounds of formula I or II have one of the structural formula III, IV, V, VI, VII or VIII,

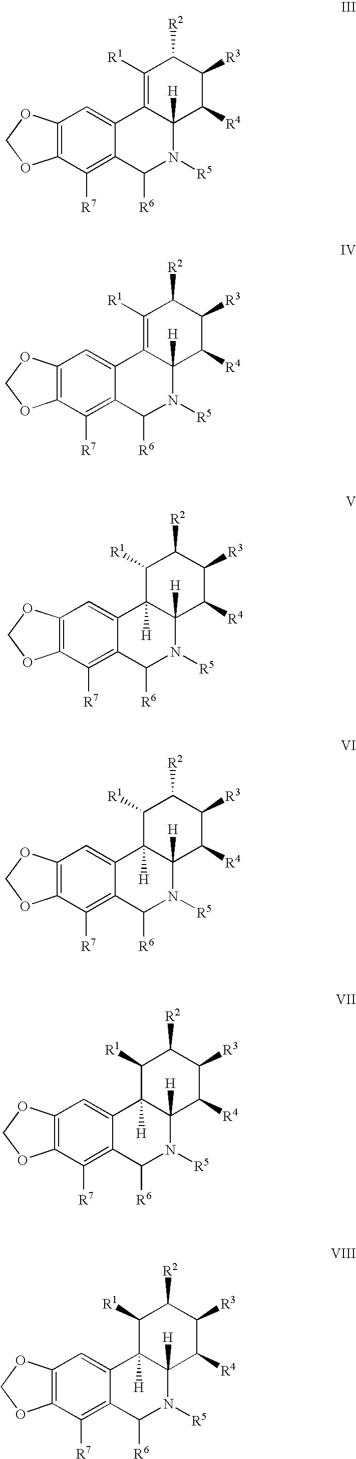

wherein R¹, R², R³, R⁴, R⁵, R⁶, and R⁷ have the same meaning as that defined herein above, with the proviso as described above.

In a particular embodiment, the present invention provides compounds of formula I, II, III, IV, V, VI, VII, or VIII, wherein R³ is a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²-amino, Het¹carbonyloxy, Het²-carbonyloxy, cycloalkylcarbonyloxy, alkoxy, aryloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano nitro, aminoalkyl, alkylamino, alkenylamino, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,

R⁴ is a group selected from hydroxyl, alkylcarbonyloxy, alkoxy, aryloxy, sulfato, arylalkyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²-amino, Het¹carbonyloxy, Het²-carbonyloxy, cycloalkylcarbonyloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, alkyloxycarbonylalkyl, carboxyalkyl, alkylaminoalkyl, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, alkenylamino, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl, and R¹, R², R⁵, R⁶, and R⁷ have the same meaning as that defined above.

In another particular embodiment, the present invention provides compounds of formula I, II, III, IV, V, VI, VII, or VIII, wherein R³ and R⁴ together with the carbon atoms to which they are attached form a dioxolane ring, said ring being optionally substituted with one or two substituents selected from alky or aryl, and R¹, R², R⁵, R⁶, and R⁷ have the same meaning as that defined hereinabove. In yet another particular embodiment, the present invention provides compounds of formula I, II, III, IV, V, VI, VII, or VIII, wherein R¹ is hydrogen or hydroxyl, or a group selected from alkyl, aryl, alkoxy, aryloxy, alkylaminocarbonyloxy, arylaminocarbonyloxy, alkylaminothiocarbonyloxy, arylaminothiocarbonyloxy, arylamino, alkylcarbonyloxy, arylcarbonyloxy, Het¹carbonyloxy, Het²-carbonyloxy, cycloalkylcarbonyloxy, aralkyl, cycloalkyl, cycloalkyloxy, Het¹, Het², Het¹alkyl, Het²alkyl, Het¹oxy, Het²oxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino, and R², R³, R⁴, R⁵, R⁶, and R⁷ have the same meaning as that defined hereinabove.

In yet another particular embodiment, the present invention provides compounds of formula I, II, III, IV, V, VI, VII, or VIII, wherein R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkenylcarbonyloxy, Het¹amino, Het²-amino, Het¹carbonyloxy, Het²-carbonyloxy, cycloalkylcarbonyloxy, alkoxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, aryloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, glucosaminyl, N-acetyl-glucosaminyl, octylglucopyranosyl, octylribofuranosyl, cyclohexylglucopyranosyl, cyclohexylxylofuranosyl, benzylglucopyranosyl, benzylarabinofuranosyl, N-acetyl-lactosaminyl, acosaminyl, amicetosyl, amylosyl, apiosyl, arcanosyl, ascarylosyl, bacillosaminyl, boivinosyl, cellotriosyl, chacotriosyl, chalcosyl, cladinosyl, colitosyl, cymarosyl, daunosaminyl, desosaminyl, d-glycero-L-gulo-heptosyl, diginosyl, dig italosyl, dig itoxosyl, evalosyl, evernitrosyl, forosaminyl, fucosaminyl, garosaminyl, hamamelosyl, isolevoglucosenonyl, kanosaminyl, kansosaminyl, lactosaminyl, lactosediaminyl, fucitolyl, maltulosyl, mannosaminyl, melezitosyl, mycaminosyl, mycarosyl, mycinosyl, mycosaminyl, noviosyl, oleandrosyl, paratosyl, perosaminyl, planteosyl, pneumosaminyl, purpurosaminyl, quinovosaminyl, quinovosyl, rhamnitolyl, rhamnosaminyl, rhodinosyl, rhodosaminyl, sarmentosyl, solatriosyl, stachyosyl, streptosyl, umbelliferosyl, trehalosaminyl, 1,6-anhydro-D-glucopyranosyl, 1-hydroxy-α-D-allopyranosyl, 2,3:5,6-di-O-isopropylidene-D-mannofuranosyl, 2-amino-2-deoxy-D-galactitolyl, 2-deoxyribosyl, 2-deoxyglucosyl, 5-amino-5-deoxy-D-glucopyranosyl, 6-deoxy-D-galactitolyl, 2-amino-2-deoxy glucosyl, 2-acetamido-2-deoxy-glucosyl, 2-amino-2-deoxy galactosyl, 2-acetamido-2-deoxy-galactosyl, 2-amino-2-deoxy mannosyl, 2-acetamido-2-deoxy-mannosyl, 2-acetamido-2-deoxy-4-O-β-D-galactosyl-D-glucosyl, 2-amino-2-deoxy-4-O-β-D-galactosyl-D-glucosyl, 6′-N-acetylglucosaminyllactosyl, 2-acetamido-2-deoxy-3-O-α-L-fucosyl-D-glucosyl, 6-O(2-acetamido-2-deoxy-β-D-glucosyl)-D-galactosyl, 2-acetamido-2-deoxy-3-O-β-D-galactosyl-D-glucosyl, 2′-acetamido-2′-deoxy-3-O-β-D-glucosyl-D-galactosyl, 3-fucosyl-D-lactosyl, or 3-fucosyl-2-acetamido-2-deoxy-4-O-β-D-galactosyl-D-glucosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thio, alkyl, haloalkyl, alkoxy, acyl, carboxyl, carboxyamino, acylamino, acetamido, haloalkoxy, alkylcarbonyl, aryl, aralkyl, Het¹, Het², cycloalkyl, carboxyl, oxo, cyano nitro, amino, aminoalkyl, alkylamino, alkenylamino, alkylamido, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, hydroxyalkyl, or phosphatyl, and R¹, R³, R⁴, R⁵, and R⁶ have the same meaning as that defined hereinabove.

In another particular embodiment, the present invention provides compounds of formula I, II, III, IV, V, VI, VII, or VIII, wherein R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkenylcarbonyloxy, Het¹amino, Het²-amino, Het¹carbonyloxy, Het²-carbonyloxy, cycloalkylcarbonyloxy, alkoxy, cycloalkyloxy, Het¹oxy, Het²oxy, Het¹alkyloxy, Het²alkyloxy, aryloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, arylcarbonyl, aryl, aralkyl, Het¹, Het², cycloalkyl, carboxyl, acyl, carboxyamino, acylamino, acetamido, alkylamido, thiol, oxo, cyano, phosphatyl, nitro, or amino, and R¹, R³, R⁴, R⁵, and R⁶ have the same meaning as that defined hereinabove.

According to a particular embodiment, the present invention provides compounds of formula I, II, III, IV, V, VI, VII, or VIII, wherein R⁵ is hydrogen or a group selected from alkyl, alkyloxycarbonylalkyl, aryloxycarbonylalkyl, aryl, aralkyl, alkenyl, alkynyl, alkylaminoalkyl, alkylcarbonyl, arylcarbonyl, cycloalkyl, Het¹alkyl, Het²alkyl, alkyloxycarbonyl, aminocarbonyl, haloalkylcarbonylaminocarbonyl, alkylcarbonylaminocarbonyl, or cycloalkylalkyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, or amino, and R¹, R², R³, R⁴, R⁷, and R⁶ have the same meaning as that defined hereinabove.

According to another particular embodiment, the present invention provides compounds of formula I, II, III, IV, V, VI, VII, or VIII, wherein R⁷ is hydrogen, hydroxyl, or a group selected from alkoxy, alkenyloxy, alkynyloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, Het¹carbonyloxy, Het²-carbonyloxy, aryloxy, sulfato, Het¹oxy, Het²oxy, alkylaminoalkyloxy, cycloalkyloxy, cycloalkylalkyloxy, Het¹alkyloxy, Het²alkyloxy, sulfato, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, acyl, carboxyamino, acylamino, acetamido, haloalkoxy, alkylcarbonyl, arylcarbonyl, aryl, aralkyl, Het¹, Het², cycloalkyl, carboxyl, oxo, cyano, phosphatyl, nitro, amino, aminoalkyl, alkylamino, arylamino, alkylamido, Het¹amino, or Het²-amino, and R¹, R², R³, R⁴, R⁵, and R⁶ have the same meaning as that defined hereinabove.

According to another particular embodiment, the present invention provides compounds of formula I, II, III, IV, V, VI, VII, or VIII, wherein R⁷ is hydrogen, hydroxyl, or a group selected from alkoxy, haloalkoxy, alkenyloxy, alkynyloxy, aryloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, Het¹carbonyloxy, Het²-carbonyloxy, Het¹oxy, Het²oxy, cycloalkyloxy, cycloalkylalkyloxy, Het¹alkyloxy, Het²alkyloxy, sulfato, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharide thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, Het¹, Het², cycloalkyl, carboxyl, oxo, cyano, nitro, phosphatyl, amino, aminoalkyl, alkylamino, arylamino, acyl, carboxyamino, acylamino, acetamido, alkylamido, Het¹amino, or Het²-amino, preferably R⁷ is hydrogen, hydroxyl, or a group selected from alkoxy, haloalkoxy, alkenyloxy, alkynyloxy, aryloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, Het¹carbonyloxy, Het²-carbonyloxy, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, carboxyl, acetamido, alkylamido, haloalkoxy, alkylcarbonyl, arylcarbonyl, aryl, aralkyl, Het¹, Het², cycloalkyl, carboxyl, oxo, cyano, nitro, phosphatyl, or amino, and

wherein R¹, R², R³, R⁴, R⁵, and R⁶ have the same meaning as that defined hereinabove. Preferred compounds according to the present invention have one of the following structural formula III1, IV1, V1, VI1, VII1, VIII1, III2, IV2, V2, VI2, VII2, VIII2, III3, IV3, V3, VI3, VII3, or VIII3, wherein R¹, R², R³, R⁴, R⁵, and R⁷ have the same meaning as that defined hereinabove,

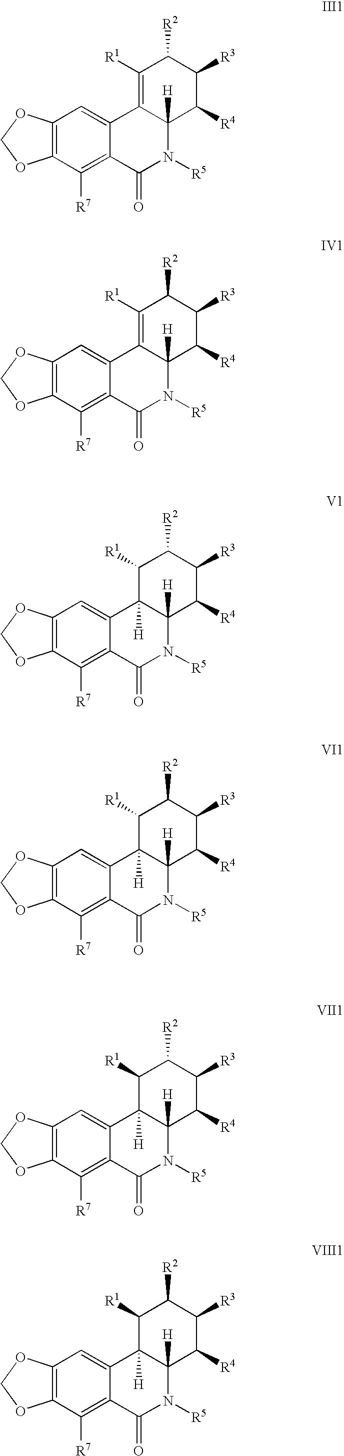

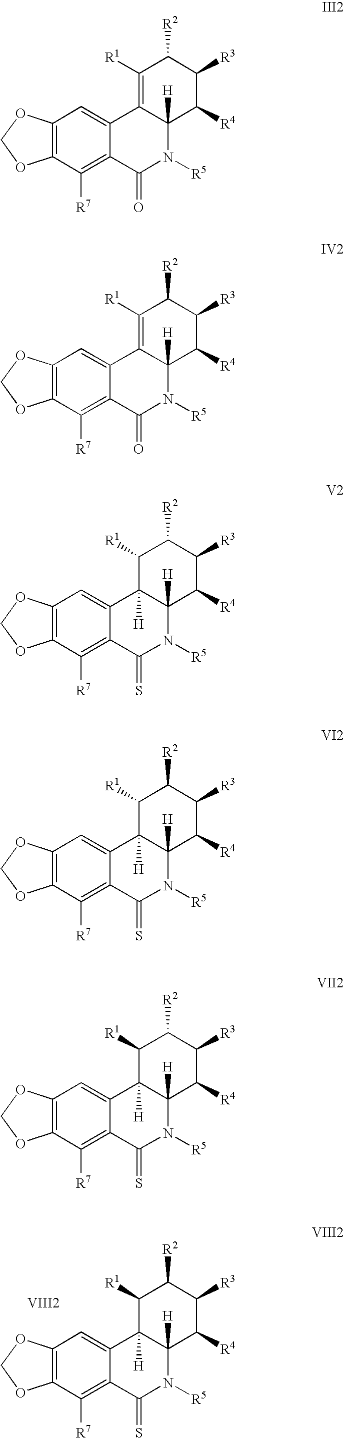

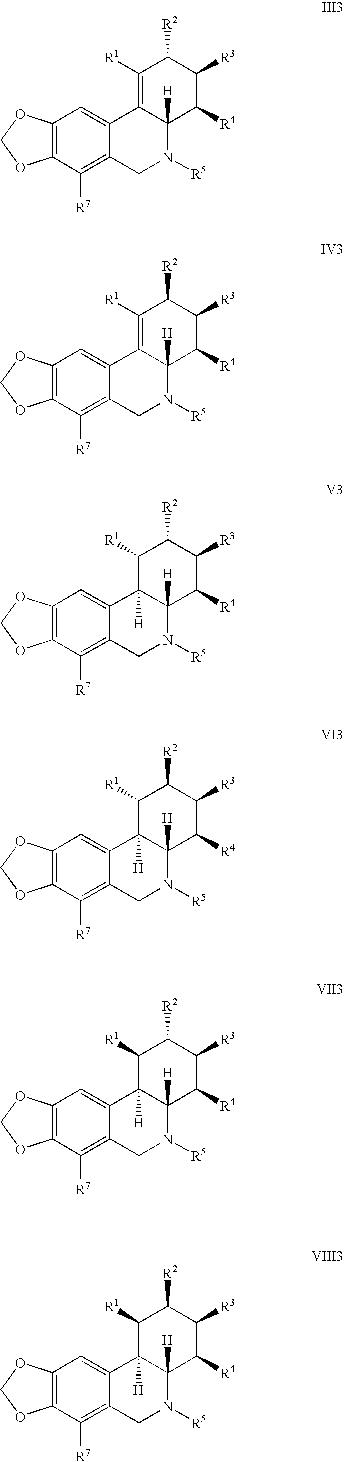

wherein R¹, R², R³, R⁴, R⁵, R⁶, and R⁷ have the same meaning as that defined hereinabove, with the proviso as described herein.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or VIII3, wherein R¹ is hydrogen, hydroxyl, or a group selected from alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, Het¹carbonyloxy, Het²-carbonyloxy, cycloalkylcarbonyloxy, aralkyl, cycloalkyl, arylamino, Het¹, Het², glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino, wherein aryl as a group or part of a group is selected from phenyl or naphthyl, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, indolinyl, tetrahydropyranyl, tetrahydrofuranyl, tetrahydrothiophenyl, thiomorpholin-4-yl, 1,3-dioxolanyl, 1,4-oxathianyl, 1,4-dithianyl, 1,3,5-trioxanyl, and morpholin-4-yl, and Het² as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl, and wherein R⁷, R², R³, R⁴, R⁵, and R⁶ have the same meaning as that defined hereinabove.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or VIII3, wherein R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkoxy, aryloxy, alkenylcarbonyloxy, Het¹amino, Het²-amino, Het¹carbonyloxy, Het²-carbonyloxy, cycloalkylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, Het¹, Het², cycloalkyl, carboxyl, oxo, cyano, phosphatyl, nitro, or amino, wherein aryl as a group or part of a group is selected from phenyl or naphthyl, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, indolinyl, tetrahydropyranyl, tetrahydrofuranyl, tetrahydrothiophenyl, thiomorpholin-4-yl, 1,3-dioxolanyl, 1,4-oxathianyl, 1,4-dithianyl, 1,3,5-trioxanyl, and morpholin-4-yl, and Het² as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl, and wherein R⁷, R¹, R³, R⁴, R⁵, and R⁶ have the same meaning as that defined hereinabove.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or VIII3, wherein R³ is a group selected from hydroxyl, alkylcarbonyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkoxy, aryloxy, alkenyloxy, alkynyloxy, cycloalkyloxy, Het¹oxy, Het²oxy, Het¹alkyloxy, Het²alkyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, or nitro, wherein aryl as a group or part of a group is selected from phenyl or naphthyl, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, indolinyl, tetrahydropyranyl, tetrahydrofuranyl, tetrahydrothiophenyl, thiomorpholin-4-yl, 1,3-dioxolanyl, 1,4-oxathianyl, 1,4-dithianyl, 1,3,5-trioxanyl, and morpholin-4-yl, and Het² as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl, and wherein R⁷, R¹, R², R⁴, R⁵, and R⁶ have the same meaning as that defined hereinabove.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or VIII3, wherein R⁴ is a group selected from hydroxyl, alkylcarbonyloxy, alkoxy, aryloxy, sulfato, arylalkyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkenyloxy, alkynyloxy, cycloalkyloxy, Het¹oxy, Het²oxy, Het¹alkyloxy, Het²alkyloxy, alkyloxycarbonylalkyl, carboxyalkyl, alkylaminoalkyl, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino, wherein aryl as a group or part of a group is selected from phenyl or naphthyl, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, indolinyl, tetrahydropyranyl, tetrahydrofuranyl, tetrahydrothiophenyl, thiomorpholin-4-yl, 1,3-dioxolanyl, 1,4-oxathianyl, 1,4-dithianyl, 1,3,5-trioxanyl, and morpholin-4-yl, and Het² as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl, and wherein R⁷, R¹, R², R³, R⁵, and R⁶ have the same meaning as that defined hereinabove.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or VIII3, wherein R⁵ is hydrogen or a group selected from alkyl, alkyloxycarbonylalkyl, aryloxycarbonylalkyl, aryl, aralkyl, alkenyl, alkynyl, cycloalkyl, Het¹alkyl, Het²alkyl, alkylcarbonyl, arylcarbonyl, alkyloxycarbonyl, aminocarbonyl, haloalkylcarbonylaminocarbonyl, alkylcarbonylaminocarbonyl, or cycloalkylalkyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, or amino, and wherein aryl as a group or part of a group is selected from phenyl or naphthyl, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, indolinyl, tetrahydropyranyl, tetrahydrofuranyl, tetrahydrothiophenyl, thiomorpholin-4-yl, 1,3-dioxolanyl, 1,4-oxathianyl, 1,4-dithianyl, 1,3,5-trioxanyl, and morpholin-4-yl, and Het² as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl, and wherein R⁷, R¹, R², R³, R⁴, and R⁶ have the same meaning as that defined hereinabove.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R⁷ is hydrogen, hydroxyl, or a group selected from alkoxy, haloalkoxy, alkenyloxy, alkynyloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, Het¹carbonyloxy, Het²-carbonyloxy, aryloxy, Het¹oxy, Het²oxy, cycloalkyloxy, cycloalkylalkyloxy, Het¹alkyloxy, Het²alkyloxy, sulfato, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, Het¹, Het², cycloalkyl, carboxyl, oxo, cyano, phosphatyl, nitro, or amino, wherein aryl as a group or part of a group is selected from phenyl or naphthyl, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, indolinyl, tetrahydropyranyl, tetrahydrofuranyl, tetrahydrothiophenyl, thiomorpholin-4-yl, 1,3-dioxolanyl, 1,4-oxathianyl, 1,4-dithianyl, 1,3,5-trioxanyl, and morpholin-4-yl, and Het² as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl, and wherein R⁵, R¹, R², R³, R⁴, and R⁶ have the same meaning as that defined hereinabove.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R³ and R⁴ together with the carbon atoms to which they are attached form a dioxolane ring, said ring being optionally substituted with one or two substituents selected from alkyl or aryl, wherein aryl as a group or part of a group is selected from phenyl or naphthyl, biphenylyl, biphenylenyl, indenyl, anthryl, indanyl, tetrahydronaphthyl, pyrenyl, and wherein R¹, R², R⁵, R⁶, and R⁷ have the same meaning as that defined hereinabove.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein

R¹ is hydrogen, hydroxyl, or a group selected from alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, Het¹carbonyloxy, Het²-carbonyloxy, cycloalkylcarbonyloxy, aralkyl, cycloalkyl, arylamino, Het¹, Het², glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino,
R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkoxy, aryloxy, alkenylcarbonyloxy, Het¹amino, Het²-amino, Het¹carbonyloxy, Het²-carbonyloxy, cycloalkylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, Het¹, Het², cycloalkyl, carboxyl, oxo, cyano, phosphatyl, nitro, or amino,
R³ is a group selected from hydroxyl, alkylcarbonyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkoxy, aryloxy, alkenyloxy, alkynyloxy, cycloalkyloxy, Het¹oxy, Het²oxy, Het¹alkyloxy, Het²alkyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, or nitro,
R⁴ is a group selected from hydroxyl, alkylcarbonyloxy, alkoxy, aryloxy, sulfato, arylalkyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkenyloxy, alkynyloxy, cycloalkyloxy, Het¹oxy, Het²oxy, Het¹alkyloxy, Het²alkyloxy, alkyloxycarbonylalkyl, carboxyalkyl, alkylaminoalkyl, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino,
R⁵ is hydrogen or a group selected from alkyl, alkyloxycarbonylalkyl, aryloxycarbonylalkyl, aryl, aralkyl, alkenyl, alkynyl, cycloalkyl, Het¹alkyl, Het²alkyl, alkylcarbonyl, arylcarbonyl, alkyloxycarbonyl, aminocarbonyl, haloalkylcarbonylaminocarbonyl, alkylcarbonylaminocarbonyl, or cycloalkylalkyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, or amino, and
R⁷ is hydrogen, hydroxyl, or a group selected from alkoxy, haloalkoxy, alkenyloxy, alkynyloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, Het¹carbonyloxy, Het²-carbonyloxy, aryloxy, Het¹oxy, Het²oxy, cycloalkyloxy, cycloalkylalkyloxy, Het¹alkyloxy, Het²alkyloxy, sulfato, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, Het¹, Het², cycloalkyl, carboxyl, oxo, cyano, phosphatyl, nitro, or amino.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein

R¹ is hydrogen, hydroxyl, or a group selected from alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, Het¹carbonyloxy, Het²-carbonyloxy, cycloalkylcarbonyloxy, aralkyl, cycloalkyl, arylamino, Het¹, Het², glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino,
R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkoxy, aryloxy, alkenylcarbonyloxy, Het¹amino, Het²-amino, Het¹carbonyloxy, Het²-carbonyloxy, cycloalkylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thiol, alkyl, haloalkyl, alkoxy, haloalkoxy, alkylcarbonyl, arylcarbonyl aryl, aralkyl, acyl, carboxyamino, acylamino, acetamido, alkylamido, Het¹, Het², cycloalkyl, carboxyl, oxo, cyano, phosphatyl, nitro, or amino,
R³ and R⁴ together with the carbon atoms to which they are attached form a dioxolane ring, said ring being optionally substituted with one or two substituents selected from alkyl or aryl,
R⁵ is hydrogen or a group selected from alkyl, alkyloxycarbonylalkyl, aryloxycarbonylalkyl, aryl, aralkyl, alkenyl, alkynyl, cycloalkyl, Het¹alkyl, Het²alkyl, cycloalkylalkyl, alkylcarbonyl, alkyloxycarbonyl, aminocarbonyl, haloalkylcarbonylaminocarbonyl, alkylcarbonylaminocarbonyl, or arylcarbonyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, or amino, and
R⁷ is hydrogen, hydroxyl, or a group selected from alkoxy, haloalkoxy, alkenyloxy, alkynyloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, Het¹carbonyloxy, Het²-carbonyloxy, aryloxy, Het¹oxy, Het²oxy, cycloalkyloxy, cycloalkylalkyloxy, Het¹alkyloxy, Het²alkyloxy, sulfato, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁹ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, Het¹, Het², cycloalkyl, carboxyl, acyl, carboxyamino, acylamino, acetamido, acetamido, oxo, cyano, phosphatyl, nitro, or amino.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein

R¹ is hydrogen, hydroxyl or a group selected from alkyl, aryl, alkoxy, aryloxy, alkylaminocarbonyloxy, arylaminocarbonyloxy, alkylaminothiocarbonyloxy, arylaminothiocarbonyloxy, aralkyl, cycloalkyl, alkylcarbonyloxy, arylcarbonyloxy, each group being optionally substituted with one or more substituents independently selected from halogen, alkyl, haloalkyl, alkoxy, haloalkoxy, alkylcarbonyl, aryl, aralkyl, Het¹, Het², cycloalkyl, or saccharyl, more preferably R¹ is hydrogen, hydroxyl or a group selected from alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, saccharyl, each group being optionally substituted with one or more substituents independently selected from halogen, alkyl, haloalkyl, alkoxy, haloalkoxy, or alkylcarbonyl,
R² is oxo or a group selected from hydroxyl, alkylcarbonyloxy, arylcarbonyloxy, Het¹carbonyloxy, Het²-carbonyloxy, cycloalkylcarbonyloxy, amino, alkylamino, arylamino, alkylaminocarbonyloxy, arylaminocarbonyloxy, alkylaminothiocarbonyloxy, arylaminothiocarbonyloxy, alkoxy, aryloxy, alkenylcarbonyloxy, Het¹amino, Het²-amino, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, idosyl, gulosyl, altrosyl, allosyl, isomaltosyl, maltosyl, lactosyl, sucrosyl, cellobiosyl, trehalosyl, gentiobiosyl, melibiosyl, isosucrosyl, raffinosyl, lactulosyl, 3-mannobiosyl, 6-mannobiosyl, 3-galactobiosyl, 4-galactobiosyl, maltotriosyl, maltotetraosyl, isomaltotriosyl, inosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, carboxyl, cyano, phosphatyl, nitro, or amino, preferably R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkoxy, aryloxy, alkenylcarbonyloxy, Het¹carbonyloxy, Het²-carbonyloxy, cycloalkylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, idosyl, gulosyl, altrosyl, allosyl, isomaltosyl, maltosyl, lactosyl, sucrosyl, cellobiosyl, trehalosyl, gentiobiosyl, melibiosyl, isosucrosyl, raffinosyl, or lactulosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, carboxyl, phosphatyl, or amino,
R³ is a group selected from hydroxyl, alkylcarbonyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkoxy, aryloxy, alkenyloxy, alkynyloxy, cycloalkyloxy, Het¹oxy, Het²oxy, Het¹alkyloxy, Het²alkyloxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, or nitro, preferably R³ is selected from hydroxyl, alkylcarbonyloxy, alkylaminocarbonyloxy, arylaminocarbonyloxy, alkylaminothiocarbonyloxy, arylaminothiocarbonyloxy, arylcarbonyloxy, Het¹carbonyloxy, Het²-carbonyloxy, or cycloalkylcarbonyloxy, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, or nitro,
R⁴ is a group selected from hydroxyl, alkylcarbonyloxy, alkoxy, aryloxy, sulfato, arylalkyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkenyloxy, alkynyloxy, cycloalkyloxy, Het¹oxy, Het²oxy, Het¹alkyloxy, Het²alkyloxy, alkyloxycarbonylalkyl, carboxyalkyl, alkylaminoalkyl, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino, preferably R⁴ is selected from hydroxyl, alkylcarbonyloxy, arylcarbonyloxy, alkoxy, sulfato, arylalkyloxy, alkylaminocarbonyloxy, arylaminocarbonyloxy, alkylaminothiocarbonyloxy, arylaminothiocarbonyloxy, Het¹carbonyloxy, Het²-carbonyloxy, alkyloxycarbonylalkyl, carboxyalkyl, alkylaminoalkyl, or cycloalkylcarbonyloxy, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, or nitro, or
R³ and R⁴ together with the carbon atoms to which they are attached form a dioxolane ring, said ring being optionally substituted with one or two substituents selected from alkyl or aryl,
R⁵ is hydrogen or a group selected from alkyl, alkyloxycarbonylalkyl, aryloxycarbonylalkyl, aryl, aralkyl, alkylcarbonyl, alkyloxycarbonyl, aminocarbonyl, haloalkylcarbonylaminocarbonyl, alkylcarbonylaminocarbonyl, arylcarbonyl, each group being optionally substituted with one or more substituents independently selected from halogen, alkyl, haloalkyl, alkoxy, haloalkoxy, aryl, carboxyl, oxo, cyano, nitro, or amino, preferably R⁵ is selected from hydrogen or a group selected from alkyl, alkyloxycarbonylalkyl, aryloxycarbonylalkyl, aryl, aralkyl, alkylcarbonyl, alkyloxycarbonyl, aminocarbonyl, haloalkylcarbonylaminocarbonyl, alkylcarbonylaminocarbonyl, arylcarbonyl, each group being optionally substituted with one or more substituents independently selected from halogen, alkyl, haloalkyl, alkoxy, haloalkoxy, aryl, carboxyl, oxo, cyano, nitro, or amino, and
R⁷ is hydrogen, hydroxyl or a group selected from alkoxy, haloalkoxy, alkenyloxy, alkynyloxy aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, aryloxy, Het¹oxy, Het²oxy, sulfato, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, cycloalkylamino, Het¹amino, Het²-amino, alkylsulfonylamino, phosphatyl, thiol, alkylthio, arylthio, alkylsulfonyl, arylsulfonyl, alkenylcarbonyloxy, —O—R⁹—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, Het¹, Het², cycloalkyl, carboxyl, acyl, carboxyamino, acylamino, acetamido, acetamido, oxo, cyano, phosphatyl, nitro, or amino, preferably R⁷ is hydrogen, hydroxyl or a group selected from alkoxy, haloalkoxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, aryloxy, sulfato, alkylaminocarbonyloxy, arylaminocarbonyloxy, alkylaminothiocarbonyloxy, arylaminothiocarbonyloxy, amino, alkylamino, alkenylamino, alkylcarbonylamino, arylcarbonylamino, formylamino, arylamino, Het¹amino, Het²-amino, alkylsulfonylamino, cycloalkylamino, thiol, alkylthio, arylthio, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, or lactulosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁹ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, Het¹, Het², cycloalkyl, carboxyl, acyl, carboxyamino, acylamino, acetamido, acetamido, oxo, cyano, phosphatyl, nitro, or amino,
wherein aryl as a group or part of a group is selected from phenyl, naphthyl, or biphenylyl, Het¹ as a group or part of a group is selected from piperidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, indolinyl, tetrahydropyranyl, tetrahydrofuranyl, tetrahydrothiophenyl, thiomorpholin-4-yl, 1,3-dioxolanyl, 1,4-oxathianyl, 1,4-dithianyl, 1,3,5-trioxanyl, and morpholin-4-yl, and Het² as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein

R¹ is hydrogen or a group selected from hydroxyl, C₁₋₆alkyl, phenyl, biphenylyl, biphenylenyl, naphthyl, indenyl, anthryl, indanyl, tetrahydronaphthyl, pyrenyl, C₁₋₆alkylcarbonyloxy, methylcyclopentylcarbonyloxy, cyclopentylmethylenecarbonyloxy, phenylcarbonyloxy, biphenylylcarbonyloxy, biphenylenylcarbonyloxy, naphthylcarbonyloxy, indenylcarbonyloxy, anthrylcarbonyloxy, indanylcarbonyloxy, piperidinylcarbonyloxy, azetidinylcarbonyloxy, imidazolinylcarbonyloxy, imidazolidinylcarbonyloxy, isoxazolinylcarbonyloxy, oxazolidinylcarbonyloxy, morpholinylcarbonyloxy, furylcarbonyloxy, thienylcarbonyloxy, pyrrolylcarbonyloxy, imidazolylcarbonyloxy, pyrazolylcarbonyloxy, isoxazolylcarbonyloxy, oxazolylcarbonyloxy, isothiazolylcarbonyloxy, thiazolylcarbonyloxy, C₃₋₈cycloalkylcarbonyloxy, C₁₋₆alkoxy, phenyloxy, biphenylyloxy, biphenylenyloxy, naphthyloxy, C₁₋₆alkylaminocarbonyloxy, phenylaminocarbonyloxy, biphenylylaminocarbonyloxy, biphenylenylaminocarbonyloxy, naphthylaminocarbonyloxy, indenylaminocarbonyloxy, phenylaminothiocarbonyloxy, biphenylylaminothiocarbonyloxy, biphenylenylaminothiocarbonyloxy, naphthylaminothiocarbonyloxy, C₁₋₆alkylaminothiocarbonyloxy, C₁₋₆alkylamino, benzyl, naphthylmethyl, indenylmethyl, anthrylmethyl, C₃₋₈cycloalkyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, furyl, thienyl, pyrrolyl, imidazolyl, pyrazolyl, isoxazolyl, oxazolyl, isothiazolyl, thiazolyl, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, C₁₋₆alkyl, chloromethyl, 1-bromoethyl, fluoromethyl, difluoromethyl, trifluoromethyl, 1,1,1-trifluoroethyl, C₁₋₆alkoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, naphthyl, indenyl, anthryl, indanyl, tetrahydronaphthyl, pyrenyl, benzyl, naphthylmethyl, indenylmethyl, anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, furyl, thienyl, pyrrolyl, imidazolyl, pyrazolyl, isoxazolyl, oxazolyl, isothiazolyl, thiazolyl, C₃₋₈cycloalkyl, carboxyl, oxo, cyano, nitro, amino, methylamino, ethylamino, n-propylamino, isopropylamino, n-butylamino, isobutylamino, sec-butylamino, tert-butylamino, n-hexylamino,
   R² is oxo or a group selected from hydroxyl, C₁₋₆alkylcarbonyloxy, methylcyclopentylcarbonyloxy, cyclopentylmethylenecarbonyloxy, phenylcarbonyloxy, biphenylylcarbonyloxy, biphenylenylcarbonyloxy, naphthylcarbonyloxy, indenylcarbonyloxy, anthrylcarbonyloxy, indanylcarbonyloxy, amino, C₁₋₆alkylamino, phenylamino, biphenylylamino, biphenylenylamino, naphthylamine, piperidinylcarbonyloxy, azetidinylcarbonyloxy, imidazolinylcarbonyloxy, imidazolidinylcarbonyloxy, isoxazolinylcarbonyloxy, oxazolidinylcarbonyloxy, morpholinylcarbonyloxy, furylcarbonyloxy, thienylcarbonyloxy, pyrrolylcarbonyloxy, imidazolylcarbonyloxy, pyrazolylcarbonyloxy, isoxazolylcarbonyloxy, oxazolylcarbonyloxy, isothiazolylcarbonyloxy, thiazolylcarbonyloxy, C₃₋₈cycloalkylcarbonyloxy, C₁₋₆alkylaminocarbonyloxy, phenylaminocarbonyloxy, biphenylylaminocarbonyloxy, biphenylenylaminocarbonyloxy, naphthylaminocarbonyloxy, C₁₋₆alkylaminothiocarbonyloxy, phenylaminothiocarbonyloxy, biphenylylaminothiocarbonyloxy, biphenylenylaminothiocarbonyloxy, naphthylaminothiocarbonyloxy, C₁₋₆alkoxy, phenyloxy, biphenylyloxy, biphenylenyloxy, naphthyloxy, C₂₋₆alkenylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, thiol, C₁₋₆alkyl, chloromethyl, 1-bromoethyl, fluoromethyl, difluoromethyl, trifluoromethyl, 1,1,1-trifluoroethyl, C₁₋₆alkoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, naphthyl, indenyl, anthryl, indanyl, tetrahydronaphthyl, pyrenyl, benzyl, naphthylmethyl, indenylmethyl, indenylmethyl, anthrylmethyl, carboxyamino, acetamido, C₁₋₆alkylamino, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, furyl, thienyl, pyrrolyl, imidazolyl, pyrazolyl, isoxazolyl, oxazolyl, isothiazolyl, thiazolyl, benzoyl, C₃₋₈cycloalkyl, carboxyl, oxo, cyano, phosphatyl, nitro, or amino,
   R³ is a group selected from hydroxyl, C₁₋₆alkylcarbonyloxy, methylcyclopentylcarbonyloxy, cyclopentylmethylenecarbonyloxy, phenylcarbonyloxy, biphenylylcarbonyloxy, biphenylenylcarbonyloxy, naphthylcarbonyloxy, indenylcarbonyloxy, anthrylcarbonyloxy, indanylcarbonyloxy, piperidinylcarbonyloxy, azetidinylcarbonyloxy, imidazolinylcarbonyloxy, imidazolidinylcarbonyloxy, isoxazolinylcarbonyloxy, oxazolidinylcarbonyloxy, morpholinylcarbonyloxy, furylcarbonyloxy, thienylcarbonyloxy, pyrrolylcarbonyloxy, imidazolylcarbonyloxy, pyrazolylcarbonyloxy, isoxazolylcarbonyloxy, oxazolylcarbonyloxy, isothiazolylcarbonyloxy, thiazolylcarbonyloxy, C₃₋₈cycloalkylcarbonyloxy, C₁₋₆alkylaminocarbonyloxy, phenylaminocarbonyloxy, biphenylylaminocarbonyloxy, biphenylenylaminocarbonyloxy, naphthylaminocarbonyloxy, C₁₋₆alkylaminothiocarbonyloxy, phenylaminothiocarbonyloxy, biphenylylaminothiocarbonyloxy, biphenylenylaminothiocarbonyloxy, naphthylaminothiocarbonyloxy, C₁₋₆alkoxy, C₂₋₆alkenyloxy, C₃₋₆cycloalkyloxy, phenyloxy, biphenylyloxy, biphenylenyloxy, naphthyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, C₁₋₆alkyl, chloromethyl, 1-bromoethyl, fluoromethyl, difluoromethyl, trifluoromethyl, 1,1,1-trifluoroethyl, C₁₋₆alkoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, naphthyl, indenyl, anthryl, indanyl, tetrahydronaphthyl, pyrenyl, benzyl, naphthylmethyl, indenylmethyl, anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, furyl, thienyl, pyrrolyl, imidazolyl, pyrazolyl, isoxazolyl, oxazolyl, isothiazolyl, thiazolyl, C₁₋₆alkylamino, C₃₋₈cycloalkyl, carboxyl, oxo, cyano, or nitro,
   R⁴ is a group selected from hydroxyl, C₁₋₆alkylcarbonyloxy, C₁₋₆alkoxy, sulfato, C₁₋₆alkyloxycarbonylC₁₋₆alkyl, carboxyC₁₋₆alkyl, C₁₋₆alkylaminoC₁₋₆alkyl, 2-phenylethoxy, 2-phenyl-1-propoxy, naphthylmethoxy, phenylcarbonyloxy, biphenylylcarbonyloxy, biphenylenylcarbonyloxy, naphthylcarbonyloxy, indenylcarbonyloxy, anthrylcarbonyloxy, indanylcarbonyloxy, piperidinylcarbonyloxy, azetidinylcarbonyloxy, imidazolinylcarbonyloxy, imidazolidinylcarbonyloxy, isoxazolinylcarbonyloxy, oxazolidinylcarbonyloxy, morpholinylcarbonyloxy, furylcarbonyloxy, thienylcarbonyloxy, pyrrolylcarbonyloxy, imidazolylcarbonyloxy, pyrazolylcarbonyloxy, isoxazolylcarbonyloxy, oxazolylcarbonyloxy, isothiazolylcarbonyloxy, thiazolylcarbonyloxy, C₃₋₈cycloalkylcarbonyloxy, 2-phenylethoxy, 2-phenyl-1-propoxy, phenyloxy, biphenylyloxy, biphenylenyloxy, naphthyloxy, pyridinium sulfato, sodium sulfato, ammonium sulfato, C₁₋₆alkylaminocarbonyloxy, phenylaminocarbonyloxy, biphenylylaminocarbonyloxy, biphenylenylaminocarbonyloxy, naphthylaminocarbonyloxy, C₁₋₆alkylaminothiocarbonyloxy, phenylaminothiocarbonyloxy, biphenylylaminothiocarbonyloxy, biphenylenylaminothiocarbonyloxy, naphthylaminothiocarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, C₁₋₆alkyl, chloromethyl, 1-bromoethyl, fluoromethyl, difluoromethyl, trifluoromethyl, 1,1,1-trifluoroethyl, C₁₋₆alkoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, naphthyl, indenyl, anthryl, indanyl, tetrahydronaphthyl, pyrenyl, benzyl, naphthylmethyl, indenylmethyl, anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, furyl, thienyl, pyrrolyl, imidazolyl, pyrazolyl, isoxazolyl, oxazolyl, isothiazolyl, thiazolyl, C₃₋₈cycloalkyl, carboxyl, oxo, cyano, nitro, amino, or C₁₋₆alkylamino, or
   R³ and R⁴ together with the carbon atoms to which they are attached form a dioxolane ring, said ring being optionally substituted with one or two substituents selected from C₁₋₆alkyl phenyl, biphenylyl, biphenylenyl, naphthyl, indenyl, anthryl, indanyl, tetrahydronaphthyl, or pyrenyl,
   R⁵ is hydrogen or a group selected from C₁₋₆alkyl, C₁₋₆alkoxycarbonyC₁₋₆alkyl, phenyl, biphenylyl, biphenylenyl, naphthyl, indenyl, anthryl, indanyl, tetrahydronaphthyl, pyrenyl, benzyl, naphthylmethyl, indenylmethyl, anthrylmethyl, C₂₋₆alkenyl, 2-phenylethyl, 2-phenyl-1-propyl, acetyl, propionyl, butyryl, valeryl, pivaloyl, phenylacetyl, phenylpropionyl, phenylbutylyl, phenylcarbonyl, biphenylylcarbonyl, biphenylenylcarbonyl, naphthylcarbonyl, aminocarbonyl, C₁₋₆alkoxycarbonyC₁₋₆alkyl, aminocarbonyl, C₁₋₆alkyloxycarbonyl, C₁₋₆alkylcarbonylaminocarbonyl, haloC₁₋₆alkylcarbonylaminocarbonyl, C₃₋₈cycloalkyl,
each group being optionally substituted with one or more substituents independently selected from fluoro, chloro, bromo, C₁₋₆alkyl, chloromethyl, 1-bromoethyl, fluoromethyl, difluoromethyl, trifluoromethyl, 1,1,1-trifluoroethyl, C₁₋₆alkoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, naphthyl, indenyl, anthryl, indanyl, tetrahydronaphthyl, pyrenyl, benzyl, naphthylmethyl, indenylmethyl, anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, furyl, thienyl, pyrrolyl, imidazolyl, pyrazolyl, isoxazolyl, oxazolyl, isothiazolyl, thiazolyl, C₃₋₈cycloalkyl, carboxyl, oxo, cyano, nitro, or amino,
   R⁶ is hydrogen, oxo or thioxo, and
   R⁷ is hydrogen or a group selected from hydroxyl, C₁₋₆alkyloxy, haloC₁₋₆alkyloxy, C₂₋₆alkenyloxy, benzyloxy, naphthylmethoxy, indenylmethoxy, anthrylmethoxy, acetoxy, propionyloxy, butyryloxy, valeryloxy, pivaloyloxy, phenylacetoxy, phenylpropionyloxy, phenylbutylyloxy, benzoyloxy, pyridinium sulfato, sodium sulfato, 4-methylbenzoxy, 2,5-dimethoxyphenyl-2-oxoethoxy, phenyloxy, biphenylyloxy, biphenylenyloxy, naphthyloxy, indenyloxy, anthryloxy, indanyloxy, tetrahydronaphthyloxy, pyrenyloxy, piperidinyloxy, azetidinyloxy, imidazolinyloxy, imidazolidinyloxy, isoxazolinyloxy, oxazolidinyloxy, isoxazolidinyloxy, thiazolidinyloxy, isothiazolidinyloxy, piperidyloxy, succinimidyloxy, 3H-indolyloxy, indolinyloxy, isoindolinyloxy, 2H-pyrrolyloxy, pyrrolinyloxy, pyrrolidinyloxy, piperazinyloxy, pyranyloxy, dihydro-2H-pyranyloxy, 4H-pyranyloxy, 3,4-dihydro-2H-pyranyloxy, triazinyloxy, cinnolinyloxy, oxetanyloxy, thietanyloxy, 3-dioxolanyloxy, 1,4-dioxanyloxy, indolinyloxy, N-formylpiperazinyloxy, morpholinyloxy, furyloxy, thienyloxy, pyrrolyloxy, imidazolyloxy, pyrazolyloxy, isoxazolyloxy, oxazolyloxy, isothiazolyloxy, thiazolyloxy, C₃₋₈cycloalkyloxy, C₁₋₆alkoxycarbonylC₁₋₆alkyloxy, hydroxycarbonylC₁₋₆alkoxy, C₁₋₆alkylaminocarbonyloxy, phenylaminocarbonyloxy, biphenylylaminocarbonyloxy, biphenylenylaminocarbonyloxy, naphthylaminocarbonyloxy, C₁₋₆alkylaminothiocarbonyloxy, phenylaminothiocarbonyloxy, biphenylylaminothiocarbonyloxy, biphenylenylaminothiocarbonyloxy, naphthylaminothiocarbonyloxy, amino, C₁₋₆alkylamino, C₂₋₆alkenylamino, C₁₋₆alkyloxyC₁₋₆alkyloxy, phenyloxymethoxy, naphthyloxymethoxy, phenyloxyethoxy, napthyloxyethoxy, phenyl-n-propoxy, naphthyloxy-n-propoxy, phenyloxy-1-methylethoxy, phenyloxy-n-butoxy, C₁₋₆alkylcarbonylamino, phenylcarbonylamino, biphenylylcarbonylamino, biphenylenylcarbonylamino, naphthylcarbonylamino, formylamino, phenylamino, biphenylylamino, biphenylenylamino, naphthylamino, C₃₋₈cycloalkylamino, C₂₋₆alkenylcarbonyloxy, phosphatyl, thiol, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
   each group being optionally substituted with one or more substituents independently selected from fluoro, chloro, bromo, thiol, chloromethyl, 1-bromoethyl, fluoromethyl, difluoromethyl, trifluoromethyl, 1,1,1-trifluoroethyl, C₁₋₆alkoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, naphthyl, indenyl, anthryl, indanyl, tetrahydronaphthyl, pyrenyl, benzyl, naphthylmethyl, indenylmethyl, anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, furyl, thienyl, pyrrolyl, imidazolyl, pyrazolyl, isoxazolyl, oxazolyl, isothiazolyl, thiazolyl, C₃₋₈cycloalkyl, carboxyl, carboxyamino, acetamido, oxo, cyano, phosphatyl, nitro, or amino.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein

R¹ is hydrogen or a group selected from hydroxyl, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, methylcarbonyloxy, ethylcarbonyloxy, n-propylcarbonyloxy, i-propylcarbonyloxy, n-butylcarbonyloxy, i-butylcarbonyloxy, t-butylcarbonyloxy, pentylcarbonyloxy, hexylcarbonyloxy, 2-, 3-, or 4-methylcyclopentylcarbonyloxy, cyclopentylmethylenecarbonyloxy, phenylcarbonyloxy, biphenylylcarbonyloxy, biphenylenylcarbonyloxy, 1-naphthylcarbonyloxy, 2-naphthylcarbonyloxy, 1-indenylcarbonyloxy, 2-indenylcarbonyloxy, 3-indenylcarbonyloxy, 1-anthrylcarbonyloxy, 2-anthrylcarbonyloxy, 4-indanylcarbonyloxy, 5-indanylcarbonyloxy, piperidinylcarbonyloxy, azetidinylcarbonyloxy, imidazolinylcarbonyloxy, imidazolidinylcarbonyloxy, isoxazolinylcarbonyloxy, oxazolidinylcarbonyloxy, morpholinylcarbonyloxy, 2- or 3-furylcarbonyloxy, 2- or 3-thienylcarbonyloxy, 1-, 2-, or 3-pyrrolylcarbonyloxy, 1-, 2-, 4-, or 5-imidazolylcarbonyloxy, 1-, 3-, 4-, or 5-pyrazolylcarbonyloxy, 3-, 4-, or 5-isoxazolylcarbonyloxy, 2-, 4-, or 5-oxazolylcarbonyloxy, 3-, 4-, or 5-isothiazolylcarbonyloxy, 2-, 4-, or 5-thiazolylcarbonyloxy, cyclopropylcarbonyloxy, cyclobutylcarbonyloxy, cyclopentylcarbonyloxy, cyclohexylcarbonyloxy, cycloheptylcarbonyloxy, cyclooctylcarbonyloxy, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, phenyloxy, biphenylyloxy, biphenylenyloxy, 1-naphthyloxy, 2-naphthyloxy, methylaminocarbonyloxy, ethylaminocarbonyloxy, n-propylaminocarbonyloxy, i-propylaminocarbonyloxy, n-butylaminocarbonyloxy, i-butylaminocarbonyloxy, t-butylaminocarbonyloxy, pentylaminocarbonyloxy, hexylaminocarbonyloxy, phenylaminocarbonyloxy, biphenylylaminocarbonyloxy, biphenylenylaminocarbonyloxy, 1-naphthylaminocarbonyloxy, 2-naphthylaminocarbonyloxy, 1-indenylaminocarbonyloxy, 2-indenylaminocarbonyloxy, 3-indenylaminocarbonyloxy, phenylaminothiocarbonyloxy, biphenylylaminothiocarbonyloxy, biphenylenylaminothiocarbonyloxy, 1-naphthylaminothiocarbonyloxy, methylaminothiocarbonyloxy, ethylaminothiocarbonyloxy, n-propylaminothiocarbonyloxy, 1-methylethylaminothiocarbonyloxy, n-butylaminothiocarbonyloxy, 1-methylpropylaminothiocarbonyloxy, 2-methylpropylaminothiocarbonyloxy, 1,1-dimethylethylaminothiocarbonyloxy, n-pentylaminothiocarbonyloxy, 1-methylbutylaminothiocarbonyloxy, 2-methylbutylaminothiocarbonyloxy, 3-methylbutylaminothiocarbonyloxy, 2,2-dimethylpropylaminothiocarbonyloxy, 1-ethylpropylaminothiocarbonyloxy, n-hexylaminothiocarbonyloxy, methylamino, ethylamino, n-propylamino, isopropylamino, n-butylamino, isobutylamino, sec-butylamino, tert-butylamino, n-hexylamino, benzyl, 1-naphthylmethyl, 2-naphthylmethyl, 1-indenylmethyl, 2-indenylmethyl, 3-indenylmethyl, 1-anthrylmethyl, 2-anthrylmethyl, cyclopropyl, cyclobutyl, cyclopentyl, cyclohexyl, cycloheptyl, cyclooctyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, 2- or 3-furyl, 2- or 3-thienyl, 1-, 2- or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloromethyl, 1-bromoethyl, fluoromethyl, difluoromethyl, trifluoromethyl, 1,1,1-trifluoroethyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, benzyl, 1-naphthylmethyl, 2-naphthylmethyl, 1-indenylmethyl, 2-indenylmethyl, 3-indenylmethyl, 1-anthrylmethyl, 2-anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, cyclopropyl, cyclobutyl, cyclopentyl, cyclohexyl, cycloheptyl, cyclooctyl, carboxyl, oxo, cyano, nitro, amino, methylamino, ethylamino, n-propylamino, isopropylamino, n-butylamino, isobutylamino, sec-butylamino, tert-butylamino, n-hexylamino,
   R² is oxo or a group selected from hydroxyl, methylcarbonyloxy, ethylcarbonyloxy, n-propylcarbonyloxy, i-propylcarbonyloxy, n-butylcarbonyloxy, i-butylcarbonyloxy, t-butylcarbonyloxy, pentylcarbonyloxy, hexylcarbonyloxy, 2-, 3-, or 4-methylcyclopentylcarbonyloxy, cyclopentylmethylenecarbonyloxy, phenylcarbonyloxy, biphenylylcarbonyloxy, biphenylenylcarbonyloxy, 1-naphthylcarbonyloxy, 2-naphthylcarbonyloxy, 1-indenylcarbonyloxy, 2-indenylcarbonyloxy, 3-indenylcarbonyloxy, 1-anthrylcarbonyloxy, 2-anthrylcarbonyloxy, 4-indanylcarbonyloxy, 5-indanylcarbonyloxy, amino, methylamino, ethylamino, n-propylamino, isopropylamino, n-butylamino, isobutylamino, sec-butylamino, tert-butylamino, n-hexylamino, phenylamino, biphenylylamino, biphenylenylamino, 1-naphthylamino, 2-naphthylamino, piperidinylcarbonyloxy, azetidinylcarbonyloxy, imidazolinylcarbonyloxy, imidazolidinylcarbonyloxy, isoxazolinylcarbonyloxy, oxazolidinylcarbonyloxy, morpholnylcarbonyloxy, 2- or 3-furylcarbonyloxy, 2- or 3-thienylcarbonyloxy, 1-, 2-, or 3-pyrrolylcarbonyloxy, 1-, 2-, 4-, or 5-imidazolylcarbonyloxy, 1-, 3-, 4-, or 5-pyrazolylcarbonyloxy, 3-, 4-, or 5-isoxazolylcarbonyloxy, 2-, 4-, or 5-oxazolylcarbonyloxy, 3-, 4-, or 5-isothiazolylcarbonyloxy, 2-, 4-, or 5-thiazolylcarbonyloxy, cyclopropylcarbonyloxy, cyclobutylcarbonyloxy, cyclopentylcarbonyloxy, cyclohexylcarbonyloxy, cycloheptylcarbonyloxy, cyclooctylcarbonyloxy, methylaminocarbonyloxy, ethylaminocarbonyloxy, n-propylaminocarbonyloxy, i-propylaminocarbonyloxy, n-butylaminocarbonyloxy, butylaminocarbonyloxy, t-butylaminocarbonyloxy, pentylaminocarbonyloxy, hexylaminocarbonyloxy, phenylaminocarbonyloxy, biphenylylaminocarbonyloxy, biphenylenylaminocarbonyloxy, 1-naphthylaminocarbonyloxy, 2-naphthylaminocarbonyloxy, methylaminothiocarbonyloxy, ethylaminothiocarbonyloxy, n-propylaminothiocarbonyloxy, 1-methylethylaminothiocarbonyloxy, n-butylaminothiocarbonyloxy, 1-methylpropylaminothiocarbonyloxy, 2-methylpropylaminothiocarbonyloxy, 1,1-dimethylethylaminothiocarbonyloxy, n-pentylaminothiocarbonyloxy, 1-methylbutylaminothiocarbonyloxy, 2-methylbutylaminothiocarbonyloxy, 3-methylbutylaminothiocarbonyloxy, 2,2-dimethylpropylaminothiocarbonyloxy, 1-ethylpropylaminothiocarbonyloxy, n-hexylaminothiocarbonyloxy, phenylaminothiocarbonyloxy, biphenylylaminothiocarbonyloxy, biphenylenylaminothiocarbonyloxy, 1-naphthylaminothiocarbonyloxy, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, phenyloxy, biphenylyloxy, biphenylenyloxy, 1-naphthyloxy, 2-naphthyloxy, ethenylcarbonyloxy, propenylcarbonyloxy, 1-butenylcarbonyloxy, 2-butenylcarbonyloxy, 3-butenylcarbonyloxy, 2-methylpropenylcarbonyloxy, 1-pentenylcarbonyloxy, 2-pentenylcarbonyloxy, 3-pentenylcarbonyloxy, 4-pentenylcarbonyloxy, 1-methyl-1-butenylcarbonyloxy, 2-methyl-1-butenylcarbonyloxy, 3-methyl-1-butenylcarbonyloxy, 1-methyl-2-butenylcarbonyloxy, 2-methyl-2-butenylcarbonyloxy, 3-methyl-2-butenylcarbonyloxy, 1-methyl-3-butenylcarbonyloxy, 2-methyl-3-butenylcarbonyloxy, 3-methyl-3-butenylcarbonyloxy, 1,1-dimethyl-2-propenylcarbonyloxy, 1,2-dimethyl-1-propenylcarbonyloxy, 1,2-dimethyl-2-propenylcarbonyloxy, 1-ethyl-1-propenylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, thiol, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloromethyl, 1-bromoethyl, fluoromethyl, difluoromethyl, trifluoromethyl, 1,1,1-trifluoroethyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, benzyl, 1-naphthylmethyl, 2-naphthylmethyl, 1-indenylmethyl, 2-indenylmethyl, 3-indenylmethyl, 1-anthrylmethyl, 2-anthrylmethyl, carboxyamino, acetamido, methylamino, ethylamino, n-propylamino, isopropylamino, n-butylamino, isobutylamino, sec-butylamino, tert-butylamino, n-hexylamino, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, benzoyl, cyclopropyl, cyclobutyl, cyclopentyl, cyclohexyl, cycloheptyl, cyclooctyl, carboxyl, oxo, cyano, phosphatyl, nitro, or amino,
   R³ is a group selected from hydroxyl, methylcarbonyloxy, ethylcarbonyloxy, n-propylcarbonyloxy, i-propylcarbonyloxy, n-butylcarbonyloxy, i-butylcarbonyloxy, t-butylcarbonyloxy, pentylcarbonyloxy, hexylcarbonyloxy, 2-, 3-, or 4-methylcyclopentylcarbonyloxy, cyclopentylmethylenecarbonyloxy, phenylcarbonyloxy, biphenylylcarbonyloxy, biphenylenylcarbonyloxy, 1-naphthylcarbonyloxy, 2-naphthylcarbonyloxy, 1-indenylcarbonyloxy, 2-indenylcarbonyloxy, 3-indenylcarbonyloxy, 1-anthrylcarbonyloxy, 2-anthrylcarbonyloxy, 4-indanylcarbonyloxy, 5-indanylcarbonyloxy, piperidinylcarbonyloxy, azetidinylcarbonyloxy, imidazolinylcarbonyloxy, imidazolidinylcarbonyloxy, isoxazolinylcarbonyloxy, oxazolidinylcarbonyloxy, morpholinylcarbonyloxy, 2- or 3-furylcarbonyloxy, 2- or 3-thienylcarbonyloxy, 1-, 2-, or 3-pyrrolylcarbonyloxy, 1-, 2-, 4-, or 5-imidazolylcarbonyloxy, 1-, 3-, 4-, or 5-pyrazolylcarbonyloxy, 3-, 4-, or 5-isoxazolylcarbonyloxy, 2-, 4-, or 5-oxazolylcarbonyloxy, 3-, 4-, or 5-isothiazolylcarbonyloxy, 2-, 4-, or 5-thiazolylcarbonyloxy, cyclopropylcarbonyloxy, cyclobutylcarbonyloxy, cyclopentylcarbonyloxy, cyclohexylcarbonyloxy, cycloheptylcarbonyloxy, cyclooctylcarbonyloxy, methylaminocarbonyloxy, ethylaminocarbonyloxy, n-propylaminocarbonyloxy, i-propylaminocarbonyloxy, n-butylaminocarbonyloxy, butylaminocarbonyloxy, t-butylaminocarbonyloxy, pentylaminocarbonyloxy, hexylaminocarbonyloxy, phenylaminocarbonyloxy, biphenylylaminocarbonyloxy, biphenylenylaminocarbonyloxy, 1-naphthylaminocarbonyloxy, 2-naphthylaminocarbonyloxy, methylaminothiocarbonyloxy, ethylaminothiocarbonyloxy, n-propylaminothiocarbonyloxy, 1-methylethylaminothiocarbonyloxy, n-butylaminothiocarbonyloxy, 1-methylpropylaminothiocarbonyloxy, 2-methylpropylaminothiocarbonyloxy, 1,1-dimethylethylaminothiocarbonyloxy, n-pentylaminothiocarbonyloxy, 1-methylbutylaminothiocarbonyloxy, 2-methylbutylaminothiocarbonyloxy, 3-methylbutylaminothiocarbonyloxy, 2,2-dimethylpropylaminothiocarbonyloxy, 1-ethylpropylaminothiocarbonyloxy, n-hexylaminothiocarbonyloxy, phenylaminothiocarbonyloxy, biphenylylaminothiocarbonyloxy, biphenylenylaminothiocarbonyloxy, 1-naphthylaminothiocarbonyloxy, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, 2-phenylethoxy, 2-phenyl-1-propoxy, ethenyloxy, propenyloxy, 1-butenyloxy, 2-butenyloxy, 3-butenyloxy, 2-methylpropenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, cyclopropyloxy, cyclobutyloxy, cyclopentyloxy, cyclohexyloxy, cycloheptyloxy, cyclooctyloxy, phenyloxy, biphenylyloxy, biphenylenyloxy, 1-naphthyloxy, 2-naphthyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloromethyl, 1-bromoethyl, fluoromethyl, difluoromethyl, trifluoromethyl, 1,1,1-trifluoroethyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, benzyl, 1-naphthylmethyl, 2-naphthylmethyl, 1-indenylmethyl, 2-indenylmethyl, 3-indenylmethyl, 1-anthrylmethyl, 2-anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, methylamino, ethylamino, n-propylamino, isopropylamino, n-butylamino, isobutylamino, sec-butylamino, tert-butylamino, n-hexylamino, cyclopropyl, cyclobutyl, cyclopentyl, cyclohexyl, carboxyl, oxo, cyano, or nitro,
   R⁴ is a group selected from hydroxyl, methylcarbonyloxy, ethylcarbonyloxy, n-propylcarbonyloxy, i-propylcarbonyloxy, n-butylcarbonyloxy, i-butylcarbonyloxy, t-butylcarbonyloxy, pentylcarbonyloxy, hexylcarbonyloxy, 2-, 3-, or 4-methylcyclopentylcarbonyloxy, cyclopentylmethylenecarbonyloxy, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, sulfato, methoxycarbonylmethyl, ethoxycarbonylmethyl, methoxycarbonylmethyl, ethoxycarbonylethyl, carboxymethyl, carboxyethyl, dimethylaminomethyl, methylaminomethyl, dimethylaminoethyl, methylaminoethyl, ethylaminomethyl, diethylaminomethyl, ethylaminoethyl, diethylaminoethyl, 2-phenylethoxy, 2-phenyl-1-propoxy, 1-naphthylmethoxy, 2-naphthylmethoxy, phenylcarbonyloxy, biphenylylcarbonyloxy, biphenylenylcarbonyloxy, 1-naphthylcarbonyloxy, 2-naphthylcarbonyloxy, 1-indenylcarbonyloxy, 2-indenylcarbonyloxy, 3-indenylcarbonyloxy, 1-anthrylcarbonyloxy, 2-anthrylcarbonyloxy, 4-indanylcarbonyloxy, 5-indanylcarbonyloxy, piperidinyl, azetidinylcarbonyloxy, imidazolinylcarbonyloxy, imidazolidinylcarbonyloxy, isoxazolinylcarbonyloxy, oxazolidinylcarbonyloxy, morpholinylcarbonyloxy, 2- or 3-furylcarbonyloxy, 2- or 3-thienylcarbonyloxy, 1-, 2-, or 3-pyrrolylcarbonyloxy, 1-, 2-, 4-, or 5-imidazolylcarbonyloxy, 1-, 3-, 4-, or 5-pyrazolylcarbonyloxy, 3-, 4-, or 5-isoxazolylcarbonyloxy, 2-, 4-, or 5-oxazolylcarbonyloxy, 3-, 4-, or 5-isothiazolylcarbonyloxy, 2-, 4-, or 5-thiazolylcarbonyloxy, cyclopropylcarbonyloxy, cyclobutylcarbonyloxy, cyclopentylcarbonyloxy, cyclohexylcarbonyloxy, cycloheptylcarbonyloxy, or cyclooctylcarbonyloxy, 2-phenylethoxy, 2-phenyl-1-propoxy, phenyloxy, biphenylyloxy, biphenylenyloxy, 1-naphthyloxy, 2-naphthyloxy, pyridinium sulfato, sodium sulfato, ammonium sulfato, methylaminocarbonyloxy, ethylaminocarbonyloxy, n-propylaminocarbonyloxy, i-propylaminocarbonyloxy, n-butylaminocarbonyloxy, i-butylaminocarbonyloxy, t-butylaminocarbonyloxy, pentylaminocarbonyloxy, hexylaminocarbonyloxy, phenylaminocarbonyloxy, biphenylylaminocarbonyloxy, biphenylenylaminocarbonyloxy, 1-naphthylaminocarbonyloxy, 2-naphthylaminocarbonyloxy, methylaminothiocarbonyloxy, ethylaminothiocarbonyloxy, n-propylaminothiocarbonyloxy, 1-methylethylaminothiocarbonyloxy, n-butylaminothiocarbonyloxy, 1-methylpropylaminothiocarbonyloxy, 2-methylpropylaminothiocarbonyloxy, 1,1-dimethylethylaminothiocarbonyloxy, n-pentylaminothiocarbonyloxy, 1-methylbutylaminothiocarbonyloxy, 2-methylbutylaminothiocarbonyloxy, 3-methylbutylaminothiocarbonyloxy, 2,2-dimethylpropylaminothiocarbonyloxy, 1-ethylpropylaminothiocarbonyloxy, n-hexylaminothiocarbonyloxy, phenylaminothiocarbonyloxy, biphenylylaminothiocarbonyloxy, biphenylenylaminothiocarbonyloxy, 1-naphthylaminothiocarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloromethyl, 1-bromoethyl, fluoromethyl, difluoromethyl, trifluoromethyl, 1,1,1-trifluoroethyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, benzyl, 1-naphthylmethyl, 2-naphthylmethyl, 1-indenylmethyl, 2-indenylmethyl, 3-indenylmethyl, 1-anthrylmethyl, 2-anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, cyclopropyl, cyclobutyl, cyclopentyl, cyclohexyl, carboxyl, oxo, cyano, nitro, amino, methylamino, ethylamino, n-propylamino, isopropylamino, n-butylamino, isobutylamino, sec-butylamino, tert-butylamino, or n-hexylamino, or
   R³ and R⁴ together with the carbon atoms to which they are attached form a dioxolane ring, said ring being optionally substituted with one or two substituents selected from methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, or 5-pyrenyl,
   R⁵ is hydrogen or a group selected from methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, ethyloxycarbonylethyl, methoxycarbonylmethyl, ethoxycarbonylmethyl, methoxycarbonylethyl, ethoxycarbonypropyl, benzyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, benzyl, 1-naphthylmethyl, 2-naphthylmethyl, 1-indenylmethyl, 2-indenylmethyl, 3-indenylmethyl, 1-anthrylmethyl, 2-anthrylmethyl, ethenyl, propenyl, 1-butenyl, 2-butenyl, 3-butenyl, 2-methylpropenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 2-phenylethyl, 2-phenyl-1-propyl, acetyl, propionyl, butyryl, valeryl, pivaloyl, phenylacetyl, phenylpropionyl, phenylbutylyl, phenylcarbonyl, biphenylylcarbonyl, biphenylenylcarbonyl, 1-naphthylcarbonyl, 2-naphthylcarbonyl, methoxycarbonylmethyl, ethoxycarbonylethyl, ethoxycarbonylmethyl, ethoxycarbonylmethyl, propoxycarbonylmethyl, methoxycarbonylpropyl, ethoxycarbonylpropyl, propoxycarbonylethyl, aminocarbonyl, methoxycarbonyl, ethoxycarbonyl, propoxycarbonyl, isopropoxycarbonyl, methylcarbonylaminocarbonyl, trichloromethylcarbonylaminocarbonyl, trifluoromethylcarbonylaminocarbonyl, tribromomethylcarbonylaminocarbonyl, cyclopropyl, cyclobutyl, cyclopentyl, or cyclohexyl,
each group being optionally substituted with one or more substituents independently selected from fluoro, chloro, bromo, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloromethyl, 1-bromoethyl, fluoromethyl, difluoromethyl, trifluoromethyl, 1,1,1-trifluoroethyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, benzyl, 1-naphthylmethyl, 2-naphthylmethyl, 1-indenylmethyl, 2-indenylmethyl, 3-indenylmethyl, 1-anthrylmethyl, 2-anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, cyclopropyl, cyclobutyl, cyclopentyl, cyclohexyl, carboxyl, oxo, cyano, nitro, or amino,
   R⁶ is hydrogen, oxo or thioxo, and
   R⁷ is hydrogen or a group selected from hydroxyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, n-hexyloxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy, chloromethoxy, 1-bromoethoxy, fluoromethoxy, difluoromethoxy, trifluoromethoxy, 1,1,1-trifluoroethoxy, ethenyloxy, propenyloxy, 1-butenyloxy, 2-butenyloxy, 3-butenyloxy, 2-methylpropenyloxy, 1-pentenyloxy, 2-pentenyloxy, 3-pentenyoxyl, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyloxy, 1,1-dimethyl-2-propenyloxy, 1,2-dimethyl-1-propenyloxy, 1,2-dimethyl-2-propenyloxy, 1-ethyl-1-propenyloxy, benzyloxy, 1-naphthylmethoxy, 2-naphthylmethoxy, 1-indenylmethoxy, 2-indenylmethoxy, 3-indenylmethoxy, 1-anthrylmethoxy, 2-anthrylmethoxy, acetoxy, propionyloxy, butyryloxy, valeryloxy, pivaloyloxy, phenylacetoxy, phenylpropionyloxy, phenylbutylyloxy, benzoyloxy, pyridinium sulfato, sodium sulfato, 4-methylbenzoxy, 2,5-dimethoxyphenyl-2-oxoethoxy, phenyloxy, biphenylyloxy, biphenylenyloxy, 1-naphthyloxy, 2-naphthyloxy, 1-indenyloxy, 2-indenyloxy, 3-indenyloxy, 1-anthryloxy, 2-anthryloxy, 9-anthryloxy, 4-indanyloxy, 5-indanyloxy, 5-tetrahydronaphthyloxy, 6-tetrahydronaphthyloxy, 7-tetrahydronaphthyloxy, 8-tetrahydronaphthyloxy, 1-pyrenyloxy, 2-pyrenyloxy, 3-pyrenyloxy, 4-pyrenyloxy, 5-pyrenyloxy, piperidinyloxy, azetidinyloxy, imidazolinyloxy, imidazolidinyloxy, isoxazolinyloxy, oxazolidinyloxy, isoxazolidinyloxy, thiazolidinyloxy, isothiazolidinyloxy, piperidyloxy, succinimidyloxy, 3H-indolyloxy, indolinyloxy, isoindolinyloxy, 2H-pyrrolyloxy, 1-pyrrolinyloxy, 2-pyrrolinyloxy, 3-pyrrolinyloxy, pyrrolidinyloxy, piperazinyloxy, pyranyloxy, dihydro-2H-pyranyloxy, 4H-pyranyloxy, 3,4-dihydro-2H-pyranyloxy, triazinyloxy, cinnolinyloxy, oxetanyloxy, thietanyloxy, 3-dioxolanyloxy, 1,4-dioxanyloxy, indolinyloxy, N-formylpiperazinyloxy, morpholinyloxy, 2- or 3-furyloxy, 2- or 3-thienyloxy, 1-, 2-, or 3-pyrrolyloxy, 1-, 2-, 4-, or 5-imidazolyloxy, 1-, 3-, 4-, or 5-pyrazolyloxy, 3-, 4-, or 5-isoxazolyloxy, 2-, 4-, or 5-oxazolyloxy, 3-, 4-, or 5-isothiazolyloxy, 2-, 4-, or 5-thiazolyloxy, cyclopropyloxy, cyclobutyloxy, cyclopentyloxy, cyclohexyloxy, methoxycarbonylmethyloxy, ethoxycarbonylethyloxy, ethoxycarbonylmethyloxy, ethoxycarbonylmethyloxy, propoxycarbonylmethyloxy, methoxycarbonylpropyloxy, ethoxycarbonylpropyloxy, propoxycarbonylethyloxy, hydroxycarbonylmethyloxy, hydroxycarbonylethyloxy, hydroxycarbonylpropyloxy, methylaminocarbonyloxy, ethylaminocarbonyloxy, n-propylaminocarbonyloxy, i-propylaminocarbonyloxy, n-butylaminocarbonyloxy, i-butylaminocarbonyloxy, t-butylaminocarbonyloxy, pentylaminocarbonyloxy, hexylaminocarbonyloxy, phenylaminocarbonyloxy, biphenylylaminocarbonyloxy, biphenylenylaminocarbonyloxy, 1-naphthylaminocarbonyloxy, 2-naphthylaminocarbonyloxy, methylaminothiocarbonyloxy, ethylaminothiocarbonyloxy, n-propylaminothiocarbonyloxy, 1-methylethylaminothiocarbonyloxy, n-butylaminothiocarbonyloxy, 1-methylpropylaminothiocarbonyloxy, 2-methylpropylaminothiocarbonyloxy, 1,1-dimethylethylaminothiocarbonyloxy, n-pentylaminothiocarbonyloxy, 1-methylbutylaminothiocarbonyloxy, 2-methylbutylaminothiocarbonyloxy, 3-methylbutylaminothiocarbonyloxy, 2,2-dimethylpropylaminothiocarbonyloxy, 1-ethylpropylaminothiocarbonyloxy, n-hexylaminothiocarbonyloxy, phenylaminothiocarbonyloxy, biphenylylaminothiocarbonyloxy, biphenylenylaminothiocarbonyloxy, 1-naphthylaminothiocarbonyloxy, amino, methylamino, ethylamino, n-propylamino, i-propylamino, n-butylamino, i-butylamino, t-butylamino, pentylamino, hexylamino, ethenylamino, propenylamino, 1-butenylamino, 2-butenylamino, 3-butenylamino, 2-methylpropenylamino, 1-pentenylamino, 2-pentenylamino, 3-pentenylamino, 4-pentenylamino, 1-methyl-1-butenylamino, 2-methyl-1-butenylamino, 3-methyl-1-butenylamino, 1-methyl-2-butenylamino, 2-methyl-2-butenylamino, 3-methyl-2-butenylamino, 1-methyl-3-butenylamino, 2-methyl-3-butenylamino, 3-methyl-3-butenylamino, 1,1-dimethyl-2-propenylamino, 1,2-dimethyl-1-propenylamino, 1,2-dimethyl-2-propenylamino, 1-ethyl-1-propenylamino, methoxymethoxy, methoxyethoxy, methoxy-n-propoxy, ethoxymethoxy, ethoxyethoxy, ethoxy-n-propoxy, n-propoxymethoxy, n-propoxyethoxy, n-propoxy-n-propoxy, 1-methylethoxymethoxy, n-butoxymethoxy, 1-methylpropoxymethoxy, 2-methylpropoxymethoxy, 1,1-dimethylethoxymethoxy, n-pentoxymethoxy, 1-methylbutoxymethoxy, 2-methylbutoxyethoxy, 3-methylbutoxymethoxy, 2,2-dimethylpropoxymethoxy, 1-ethylpropoxymethoxy, phenyloxymethoxy, 1-naphthyloxymethoxy, 2-napthtyloxymethoxy, phenyloxyethoxy, 1-napthyloxyethoxy, 2-napthyloxyethoxy, phenyl-n-propoxy, 1-naphthyloxy-n-propoxy, 2-naphthyloxy-n-propoxy, phenyloxy-1-methylethoxy, phenyloxy-n-butoxy, methylcarbonylamino, ethylcarbonylamino, n-propylcarbonylamino, i-propylcarbonylamino, n-butylcarbonylamino, i-butylcarbonylamino, t-butylcarbonylamino, pentylcarbonylamino, hexylcarbonylamino, phenylcarbonylamino, biphenylylcarbonylamino, biphenylenylcarbonylamino, 1-naphthylcarbonylamino, 2-naphthylcarbonylamino, formylamino, phenylamino, biphenylylamino, biphenylenylamino, 1-naphthylamino, 2-naphthylamino, cyclopropylamino, cyclobutylamino, cyclopentylamino, cyclohexylamino, cycloheptylamino, cyclooctylamino, ethenylcarbonyloxy, propenylcarbonyloxy, 1-butenylcarbonyloxy, 2-butenylcarbonyloxy, 3-butenylcarbonyloxy, 2-methylpropenylcarbonyloxy, 1-pentenylcarbonyloxy, 2-pentenylcarbonyloxy, 3-pentenylcarbonyloxy, 4-pentenylcarbonyloxy, 1-methyl-1-butenylcarbonyloxy, 2-methyl-1-butenylcarbonyloxy, 3-methyl-1-butenylcarbonyloxy, 1-methyl-2-butenylcarbonyloxy, 2-methyl-2-butenylcarbonyloxy, 3-methyl-2-butenylcarbonyloxy, 1-methyl-3-butenylcarbonyloxy, 2-methyl-3-butenylcarbonyloxy, 3-methyl-3-butenylcarbonyloxy, 1,1-dimethyl-2-propenylcarbonyloxy, 1,2-dimethyl-1-propenylcarbonyloxy, 1,2-dimethyl-2-propenylcarbonyloxy, 1-ethyl-1-propenylcarbonyloxy, thiol, methylthio, ethylthio, n-propylthio, isopropylthio, n-butylthio, isobutylthio, sec-butylthio, tert-butylthio, n-hexylthio, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
each group being optionally substituted with one or more substituents independently selected from fluoro, chloro, bromo, thiol, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, chloromethyl, 1-bromoethyl, fluoromethyl, difluoromethyl, trifluoromethyl, 1,1,1-trifluoroethyl, methoxy, ethoxy, n-propoxy, 1-methylethoxy, n-butoxy, 1-methylpropoxy, 2-methylpropoxy, 1,1-dimethylethoxy, n-pentoxy, 1-methylbutoxy, 2-methylbutoxy, 3-methylbutoxy, 2,2-dimethylpropoxy, 1-ethylpropoxy, trifluoromethoxy, difluoromethoxy, fluoromethoxy, acetyl, propionyl, butyryl, valeryl, pivaloyl, phenylacetyl, phenylpropionyl, phenylbutylyl, phenyl, biphenylyl, biphenylenyl, 1-naphthyl, 2-naphthyl, 1-indenyl, 2-indenyl, 3-indenyl, 1-anthryl, 2-anthryl, 9-anthryl, 4-indanyl, 5-indanyl, 5-tetrahydronaphthyl, 6-tetrahydronaphthyl, 7-tetrahydronaphthyl, 8-tetrahydronaphthyl, 1-pyrenyl, 2-pyrenyl, 3-pyrenyl, 4-pyrenyl, 5-pyrenyl, benzyl, 1-naphthylmethyl, 2-naphthylmethyl, 1-indenylmethyl, 2-indenylmethyl, 3-indenylmethyl, 1-anthrylmethyl, 2-anthrylmethyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, piperazinyl, pyranyl, dihydro-2H-pyranyl, 4H-pyranyl, 3,4-dihydro-2H-pyranyl, triazinyl, cinnolinyl, oxetanyl, thietanyl, 3-dioxolanyl, 1,4-dioxanyl, indolinyl, N-formylpiperazinyl, morpholinyl, 2- or 3-furyl, 2- or 3-thienyl, 1-, 2-, or 3-pyrrolyl, 1-, 2-, 4-, or 5-imidazolyl, 1-, 3-, 4-, or 5-pyrazolyl, 3-, 4-, or 5-isoxazolyl, 2-, 4-, or 5-oxazolyl, 3-, 4-, or 5-isothiazolyl, 2-, 4-, or 5-thiazolyl, cyclopropyl, cyclobutyl, cyclopentyl, cyclohexyl, carboxyl, carboxyamino, acetamido, oxo, cyano, phosphatyl, nitro, or amino.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R¹ is hydrogen, hydroxyl, alkyl, aryl, alkoxy, aryloxy, glucosyl, fructosyl, galactosyl, mannosyl, rhamnosyl, fucosyl, arabinosyl, isomaltosyl, maltosyl, lactosyl, sucrosyl, cellobiosyl, trehalosyl, isosucrosyl, lactulosyl, mannobiosyl, galactobiosyl, maltotriosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, and R², R³, R⁴, R⁵, R⁶, and R⁷ have the same meaning as that defined herein.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R² is oxo, hydroxyl, alkylcarbonyloxy, arylcarbonyloxy, amino, alkylamino, alkylaminocarbonyloxy, alkoxy, aryloxy, glucosyl, fructosyl, galactosyl, mannosyl, rhamnosyl, fucosyl, arabinosyl, isomaltosyl, maltosyl, lactosyl, sucrosyl, cellobiosyl, trehalosyl, isosucrosyl, lactulosyl, mannobiosyl, galactobiosyl, maltotriosyl, maltotetraosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, and R¹, R³, R⁴, R⁵, R⁶, and R⁷ have the same meaning as that defined herein.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R³ is a group selected from hydroxyl, alkylcarbonyloxy, alkylaminocarbonyloxy, alkoxy, aryloxy, glucosyl, fructosyl, galactosyl, mannosyl, rhamnosyl, fucosyl, arabinosyl, isomaltosyl, maltosyl, lactosyl, sucrosyl, cellobiosyl, melibiosyl, trehalosyl, isosucrosyl, lactulosyl, mannobiosyl, galactobiosyl, maltotriosyl, maltotetraosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, and R¹, R², R⁴, R⁵, R⁶, and R⁷ have the same meaning as that defined herein.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R⁴ is a group selected from hydroxyl, alkylcarbonyloxy, alkoxy, aryloxy, sulfato, glucosyl, fructosyl, galactosyl, mannosyl, rhamnosyl, fucosyl, arabinosyl, isomaltosyl, maltosyl, lactosyl, sucrosyl, cellobiosyl, trehalosyl, isosucrosyl, lactulosyl, mannobiosyl, galactobiosyl, maltotriosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, and R¹, R², R³, R⁵, R⁶, and R⁷ have the same meaning as that defined herein.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R⁵ is a group selected from hydrogen, aralkyl, alkyl, alkyloxycarbonylalkyl, aryloxycarbonylalkyl, aryl, alkylcarbonyl, aminocarbonyl, and R¹, R², R³, R⁴, R⁶, and R⁷ have the same meaning as that defined herein.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein R⁷ is a group selected from hydrogen, hydroxyl, alkoxy, haloalkoxy, aralkyloxy, alkylcarbonyloxy, haloalkylcarbonyloxy, arylcarbonyloxy, haloarylcarbonyloxy, arylcarbonylalkyloxy, carboxyalkyloxy, alkyloxycarbonylalkyloxy, haloalkoxycarbonylalkyloxy, aryloxycarbonylalkyloxy, hydroxycarbonylalkyloxy, aryloxy, alkylaminocarbonyloxy, arylaminocarbonyloxy, amino, alkylamino, glucosyl, fructosyl, galactosyl, mannosyl, rhamnosyl, fucosyl, isomaltosyl, maltosyl, lactosyl, sucrosyl, isosucrosyl, lactulosyl, mannobiosyl, galactobiosyl, maltotriosyl, maltotetraosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, and R¹, R², R³, R⁴, R⁶, and R⁵ have the same meaning as that defined herein.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or V III3, wherein

R¹ is a group selected from hydrogen, hydroxyl, alkyl, aryl, alkoxy, aryloxy, glucosyl, fructosyl, galactosyl, mannosyl, rhamnosyl, fucosyl, arabinosyl, isomaltosyl, maltosyl, lactosyl, sucrosyl, cellobiosyl, trehalosyl, isosucrosyl, lactulosyl, mannobiosyl, galactobiosyl, maltotriosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
R² is a group selected from oxo, hydroxyl, alkylcarbonyloxy, arylcarbonyloxy, amino, alkylamino, alkylaminocarbonyloxy, alkoxy, aryloxy, glucosyl, fructosyl, galactosyl, mannosyl, rhamnosyl, fucosyl, arabinosyl, isomaltosyl, maltosyl, lactosyl, sucrosyl, cellobiosyl, trehalosyl, isosucrosyl, lactulosyl, mannobiosyl, galactobiosyl, maltotriosyl, maltotetraosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
R³ is a group selected from hydroxyl, alkylcarbonyloxy, alkylaminocarbonyloxy, alkoxy, aryloxy, glucosyl, fructosyl, galactosyl, mannosyl, rhamnosyl, fucosyl, arabinosyl, isomaltosyl, maltosyl, lactosyl, sucrosyl, cellobiosyl, melibiosyl, trehalosyl, isosucrosyl, lactulosyl, mannobiosyl, galactobiosyl, maltotriosyl, maltotetraosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
R⁴ is a group selected from hydroxyl, alkylcarbonyloxy, alkoxy, aryloxy, sulfato, glucosyl, fructosyl, galactosyl, mannosyl, rhamnosyl, fucosyl, arabinosyl, isomaltosyl, maltosyl, lactosyl, sucrosyl, cellobiosyl, trehalosyl, isosucrosyl, lactulosyl, mannobiosyl, galactobiosyl, maltotriosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
or R³ and R⁴ together with the carbon atoms to which they are attached form a dioxolane ring, said ring being optionally substituted with one or two alkyl substituents,
R⁵ is a group selected from hydrogen, aralkyl, alkyl, alkyloxycarbonylalkyl, aryloxycarbonylalkyl, aryl, alkylcarbonyl, aminocarbonyl, and
R⁷ is a group selected from hydrogen, hydroxyl, haloalkoxy, alkoxy, aralkyloxy, haloalkycarbonyloxy, alkylcarbonyloxy, haloarylcarbonyloxy, arylcarbonyloxy, haloarylcarbonylalkyloxy, arylcarbonylalkyloxy, carboxyalkyloxy, alkyloxycarbonylalkyloxy, aryloxycarbonylalkyloxy, hydroxycarbonylalkyloxy, aryloxy, alkylaminocarbonyloxy, arylaminocarbonyloxy, amino, alkylamino, glucosyl, fructosyl, galactosyl, mannosyl, rhamnosyl, fucosyl, isomaltosyl, maltosyl, lactosyl, sucrosyl, isosucrosyl, lactulosyl, mannobiosyl, galactobiosyl, maltotriosyl, maltotetraosyl, isomaltotriosyl, mannotriosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof.

Preferred compounds according to the present invention have one of the following structural formula I, II, III, IV, V, VI, VII, VIII, III1, IV1, V1, VI1, VII1, VIII1, III2, IV2, V2, VI2, VII2, VIII2, III3, IV3, V3, VI3, VII3, or VIII3, wherein

R¹ is a group selected from hydrogen, hydroxyl, alkoxy, aryloxy, preferably R¹ is hydrogen,
R² is a group selected from oxo, hydroxyl, alkylcarbonyloxy, arylcarbonyloxy, amino, alkylamino, alkylaminocarbonyloxy, glucosyl, galactosyl, mannosyl, rhamnosyl, lactosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
R³ is a group selected from hydroxyl, alkylcarbonyloxy, alkylaminocarbonyloxy, alkoxy, aryloxy, glucosyl, galactosyl, mannosyl, rhamnosyl, lactosyl, sucrosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
R⁴ is a group selected from hydroxyl, alkylcarbonyloxy, alkoxy, aryloxy, sulfato, glucosyl, galactosyl, mannosyl, rhamnosyl, maltosyl, lactosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof,
R³ and R⁴ together with the carbon atoms to which they are attached form a dioxolane ring, said ring being optionally substituted with one or two alkyl substituents,
R⁵ is a group selected from hydrogen, aralkyl, alkyl, alkyloxycarbonylalkyl, aryloxycarbonylalkyl, aryl, alkylcarbonyl, aminocarbonyl,
R⁷ is a group selected from hydrogen, hydroxyl, alkoxy, haloalkoxy aralkyloxy, haloaralkoxy, alkylcarbonyloxy, arylcarbonyloxy, haloalkylcarbonyloxy, haloarylcarbonyloxy, carboxyalkyloxy, alkyloxycarbonylalkyloxy, glucosyl, galactosyl, mannosyl, rhamnosyl, maltosyl, lactosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form of a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters, and the like. Such salts, hydrates, solvates, etc., and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087, and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides, and iodides; dialkyl sulfates like dimethyl, diethyl, dibutyl; and diamyl sulfates, long chain halides such as decyl, lauryl, myristyl, and stearyl chlorides, bromides, and iodides, aralkyl halides like benzyl and phenethyl-bromides, and others. Other pharmaceutically acceptable salts include the sulfate salt ethanolate and sulfate salts.

The compounds of the invention are particularly advantageous since while being anti-proliferative and anti-migratory for cancer cells, the compounds according to the invention exhibit a low toxicity level on healthy cells. The terms “toxicity” or “toxic effects” as used herein refer to the detrimental effect(s) a compound may have on healthy cells, tissues, or organs. The toxicity level of the compounds according to the invention is surprisingly low. The compounds according to the invention combine the essential features of a good cytotoxic activity and a low level of toxicity.

Consequently the compounds according to the invention may be used in pharmaceutical compositions for the treatment of various cancers. In addition, because they have a low level of toxicity the compounds according to the invention may be used during longer periods of treatments. Due to these interesting properties; in particular the antiproliferative properties and the low level of toxicity, the compounds according to the invention are particularly suitable for use as a medicament, preferably in the treatment of cancer. Therefore, in another embodiment, the invention relates to compounds according to the invention for use as a medicament. The invention also encompasses the use of at least one compound of formula I or II as defined above, for the preparation of a medicament for treating cancer.

In another embodiment, the present invention relates to a pharmaceutical composition comprising a pharmaceutically acceptable excipient and a therapeutic amount of at least one compound according to the invention. The invention also encompasses the use of a pharmaceutical composition comprising a pharmaceutically acceptable excipient and a therapeutic amount of at least one compound according to the invention, for the preparation of a medicament for treating cancer.

The term “therapeutically effective amount” or “effective amount” as used herein refers to the quantity of compound or pharmaceutical composition that elicits the biological or medicinal response in a tissue, system, animal, or human that is being sought by a researcher, veterinarian, medical doctor or other clinician, which includes alleviation of the symptoms of the cancer being treated. In particular, these terms refer to the quantity of compound or pharmaceutical composition according to the invention which is necessary to prevent, cure, ameliorate, or at least minimize the clinical impairment, symptoms, or complications associated with cancer in either a single or multiple doses.

The pharmaceutical composition can be prepared in a manner known per se to one of skill in the art. For this purpose, at least one compound having formula I or II, one or more solid or liquid pharmaceutical excipients and, if desired, in combination with other pharmaceutical active compounds, are brought into a suitable administration form or dosage form which can then be used as a pharmaceutical in human medicine or veterinary medicine.

By means of non-limiting examples, such a formulation may be in a form suitable for oral administration, for parenteral administration (such as by intravenous, intramuscular, or subcutaneous injection, or intravenous infusion), for topical administration (including ocular), for administration by inhalation, by a skin patch, by an implant, by a suppository, etc. Such suitable administration forms—which may be solid, semi-solid, or liquid, depending on the manner of administration—as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087, and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying, and suspending agents, dispersing agents, desintegrants, bulking agents, fillers, preserving agents, sweetening agents, flavoring agents, flow regulators, release agents, etc. The compositions may also be formulated so as to provide rapid, sustained, or delayed release of the active compound(s) contained therein, for example using liposomes or hydrophilic polymeric matrices based on natural gels or synthetic polymers.

In order to enhance the solubility and/or the stability of the compounds of a pharmaceutical composition according to the invention, it can be advantageous to employ α-, β-, or γ-cyclodextrins or their derivatives. In addition, co-solvents such as alcohols may improve the solubility and/or the stability of the compounds. In the preparation of aqueous compositions, addition of salts of the compounds of the invention are obviously more suitable due to their increased water solubility.

Appropriate cyclodextrins are α-, β-, or γ-cyclodextrins (CDs) or ethers and mixed ethers thereof wherein one or more of the hydroxyl groups of the anhydroglucose units of the cyclodextrin are substituted with alkyl, particularly methyl, ethyl, or isopropyl, e.g. randomly methylated β-CD; hydroxyalkyl, particularly hydroxyethyl, hydroxypropyl, or hydroxybutyl; carboxyalkyl, particularly carboxymethyl, or carboxyethyl; alkylcarbonyl, particularly acetyl; alkyloxycarbonylalkyl or carboxyalkyloxyalkyl, particularly carboxymethoxypropyl or carboxyethoxypropyl; alkylcarbonyloxyalkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD). The term mixed ether denotes cyclodextrin derivatives wherein at least two cyclodextrin hydroxyl groups are etherified with different groups such as, for example, hydroxypropyl and hydroxyethyl. An interesting way of formulating the compounds in combination with a cyclodextrin or a derivative thereof has been described in EP-A-721,331. Although the formulations described therein are with antifungal active ingredients, they are equally interesting for formulating the compounds. Said formulations may also be rendered more palatable by adding pharmaceutically acceptable sweeteners and/or flavors.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered. The term “a solid dispersion” also comprises dispersions that are less homogenous throughout than solid solutions. Such dispersions are not chemically and physically uniform throughout or comprise more than one phase.

The water-soluble polymer is conveniently a polymer that has an apparent viscosity of 1 to 100 mPa·s when dissolved in a 2% aqueous solution at 20° C. solution. Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

It may further be convenient to formulate the compounds of the invention in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Said beads comprise (a) a central, rounded, or spherical core, (b) a coating film of a hydrophilic polymer and an antiretroviral agent and (c) a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The preparations may be prepared in a manner known per se, which usually involves mixing the at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule, or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300, or 400 mg per unit dosage.

As indicated above, due to the favourable anti-proliferative and anti-migratory properties on cancer cells and the low level of toxicity, the compounds of the invention are particularly useful in the treatment of individuals suffering from cancer. The compounds are also particularly useful in the prevention of metastatis.

The term “anti-migratory” as used herein refers to the ability of a compound of the invention to stop the migration of cells away from the neoplastic tumor tissue and thus to reduce the colonization of new tissues by these cells.

The term “treating” as used herein includes treating any one or more of the conditions underlying or characteristic of cancer. Treatment of cancer means administration of a medicament with the result that cancer is reduced or the patient is cured.

The compounds of the invention may be especially used in (the preparation of a medicament for) the treatment of cancers such as but not limited to leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma.

Preferably, said cancer is selected from non-small cell lung cancer, CNS cancer, melanoma, ovarian cancer, kidney cancer, prostate cancer, breast cancer, colon cancer, bladder cancer, sarcoma, pancreatic cancer, colorectal cancer, head and neck cancer, liver cancer, stomach cancer, oesophageal cancer, or lymphoma.

In an embodiment, the compounds can be used for the treatment of glioma. Said glioma can be in the brain but also in the spinal cord or any other part of the CNS, such as the optic nerves. For example, the compounds can be therefore used for (the preparation of a medicament for) treating ependymomas, astrocytomas, oligodendrogliomas, or mixed gliomas such as oligoastrocytomas. For example, the compounds can be used for (the preparation of a medicament for) treating astrocytomas including but not limited to pilocytic astrocytoma, diffuse astrocytoma, anaplastic (malignant) astrocytoma, and glioblastoma multiforme.

Accordingly, the present invention provides a method for the treatment of cancer comprising administering to an individual an effective amount of at least one compound of formula I or II as defined above as an active ingredient, such that the cancer is treated. By way of example, in an embodiment of the invention, cancer is treated in a subject in need of treatment by administering to the subject a therapeutically effective amount of at least one compound of formula I or II, effective to treat the cancer. The subject is preferably a mammal (e.g., humans, domestic animals, and commercial animals, including cows, dogs, monkeys, mice, pigs, and rats), and is most preferably a human.

For these purposes, the compounds or the pharmaceutical compositions of the present invention may be administered orally, parenterally, i.e. including subcutaneous injections, intravenous, intramuscular, intrasternal injection, or infusion techniques, by inhalation spray, or rectally, in dosage unit formulations containing conventional non-toxic pharmaceutically acceptable carriers, adjuvants, and vehicles. The at least one compound of the invention will generally be administered in an “effective amount”, by which is meant any amount of a compound of the Formula I or II above that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight, more often between 0.1 and 500 mg, such as between 1 and 250 mg, for example about 5, 10, 20, 50, 100, 150, 200, or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778, U.S. Pat. No. 6,369,086, U.S. Pat. No. 6,369,087, and U.S. Pat. No. 6,372,733, and the further prior art mentioned above, as well as to the standard handbooks, such as the latest edition of Remington's Pharmaceutical Sciences.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

Essentially, the primary modes of treatment of solid tumor cancers comprise surgery, radiation therapy, and chemotherapy, separately and in combination. The compounds according to the invention are suitable for use in combination with these medicinal techniques. The compounds of the invention may be useful in increasing the sensitivity of tumor cells to radiation in radiotherapy and also in potentiating or enhancing damage to tumors by chemotherapeutic agents. The compounds and their pharmaceutically acceptable salts and/or solvates may also be useful for sensitizing multidrug-resistant tumor cells. The compounds according to the invention are useful therapeutic compounds for administration in conjunction with DNA-damaging cytotoxic drugs or radiation used in radiotherapy to potentiate their effect.

In another embodiment of the method of the invention, the administration may be performed with food, e.g., a high-fat meal. The term “with food” means the consumption of a meal either during or no more than about one hour before or after administration of a pharmaceutical composition according to the invention.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate, and lactose and/or other excipients, binders, extenders, disintegrants, diluents, and lubricants known in the art.

The oral administration of a pharmaceutical composition comprising at least one compound according to the invention, or a pharmaceutically acceptable salt or ester and/or solvate thereof, is suitably accomplished by uniformly and intimately blending together a suitable amount of said compound in the form of a powder, optionally also including a finely divided solid carrier, and encapsulating the blend in, for example, a hard gelatin capsule. The solid carrier can include one or more substances, which act as binders, lubricants, disintegrating agents, coloring agents, and the like. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Oral administration of a pharmaceutical composition comprising at least one compound according to the invention, or a pharmaceutically acceptable salt or ester and/or solvate thereof can also be accomplished by preparing capsules or tablets containing the desired amount of said compound, optionally blended with a solid carrier as described above. Compressed tablets containing the pharmaceutical composition of the invention can be prepared by uniformly and intimately mixing the active ingredient with a solid carrier such as described above to provide a mixture having the necessary compression properties, and then compacting the mixture in a suitable machine to the shape and size desired. Molded tablets maybe made by molding in a suitable machine, a mixture of powdered compound moistened with an inert liquid diluent.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions, or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compound of the invention, if desired with the substances customary therefor such as solubilizers, emulsifiers, or further auxiliaries, are brought into solution, suspension, or emulsion. The compounds of the invention can also be lyophilized and the lyophilizates obtained used, for example, for the production of injection or infusion preparations. Suitable solvents are, for example, water, physiological saline solution, or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents, or solvents, such as mannitol, 1,3-butanediol, water, Ringer's solution, or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters, or polyethylene glycols, which are solid at ordinary temperatures, but liquidify and/or dissolve in the rectal cavity to release the drug.

The pharmaceutical compositions of this invention can be administered to humans in dosage ranges specific for each compound comprised in said compositions. The compounds comprised in said composition can be administered together or separately.

It will be understood, however, that specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The following examples are meant to illustrate the present invention. These examples are presented to exemplify the invention and are not to be considered as limiting the scope of the invention.

### EXAMPLES

The practice of the present invention will employ, unless otherwise indicated, conventional techniques of synthetic organic chemistry, biological testing, and the like, which are within the skill of the art. Such techniques are explained fully in the literature.

### Example 1

### Non-Limiting Examples of Compounds According to the Invention

As used in Tables 1 to 6 the term “*OAc” (acetoxy) refers to “.O—CO-methyl”, the term “.OBz” (benzoyloxy) refers to “.OCO-phenyl” and the term “Bz” (benzoyl) refers to “.CO-phenyl”.

TABLE 1

| | |
|---|---|
| | III |
| 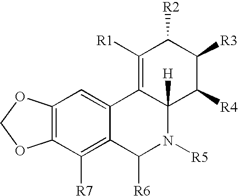 | |

| | Com- | | | | | | | |
|---|---|---|---|---|---|---|---|---|
| Name | pound | R¹ | R² | R³ | R⁴ | R⁵ | R⁶ | R⁷ |
| (2S,3R,4S,4aR)-2,3,7-trihydroxy- | 1 | H | *OH | *OH | *OSO₃Na | H | ═O | *OH |
| (1,3)dioxolo(4,5-j)-6-oxo-2,3,4,4a,5,6- | | | | | | | | |
| hexahydrophenanthridin-4-yl sodium | | | | | | | | |
| sulfate | | | | | | | | |
| ethyl ({(2S,3R,4S,4aR)-2,3,4- | 2 | H | *OAc | *OAc | *OAc | H | ═O | *OCH₂CO₂Et |
| tris[(methylcarbonyl)oxy]- | | | | | | | | |
| (1,3)dioxolo(4,5-j)-6-oxo-2,3,4,4a,5,6- | | | | | | | | |
| hexahydrophenanthridin--7- | | | | | | | | |
| yl}oxy)acetate | | | | | | | | |
| ethyl [(2S,3R,4S,4aR)-2,3,4- | 3 | H | OAc | OAc | OAc | *CH₂CO₂Et | ═O | *OCH₂CO₂Et |
| tris[(methylcarbonyl)oxy]- | | | | | | | | |
| (1,3)dioxolo(4,5-j)-7-(2-ethoxy-2- | | | | | | | | |
| oxoethoxy)-6-oxo-2,3,4,4a,5,6- | | | | | | | | |
| hexahydrophenanthridin--5(3H)- | | | | | | | | |
| yl]acetate | | | | | | | | |
| (2S,3R,4S,4aR)-7-hydroxy- | 4 | H | *OAc | *OAc | *OAc | H | ═S | OH |
| (1,3)dioxolo(4,5-j)-6-thioxo-2,3,4,4a,5,6- | | | | | | | | |
| hexahydrophenanthridin--2,3,4-triyl | | | | | | | | |
| triacetate | | | | | | | | |
| methyl {[(2S,3R,4S,4aR)-2,3,4- | 5 | H | OH | OH | OH | H | ═O | *OCH₂CO₂Me |
| trihydroxy-(1,3)dioxolo(4,5-j)-6-oxo- | | | | | | | | |
| 3,4,4a,5-tetrahydrophenanthridin-6(2H)- | | | | | | | | |
| 7-yl]oxy}acetate | | | | | | | | |
| {[(2S,3R,4S,4aR)-2,3,4-trihydroxy- | 6 | H | OH | OH | OH | H | ═O | *OCH₂CO₂H |
| (1,3)dioxolo(4,5-j)-6-oxo-3,4,4a,5- | | | | | | | | |
| tetrahydrophenanthridin-6(2H)-7- | | | | | | | | |
| yl]oxy}acetic acid | | | | | | | | |
| (2S,3R,4S,4aR)-7-[(2- naphthylmethoxy]-(1,3)dioxolo(4,5-j)-6- oxo-2,3,4,4a,5,6- hexahydrophenanthridin-2,3,4-triyl triacetate | 7 | H | *OAc | *OAc | *OAc | H | ═O | 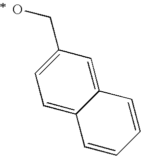 |
| (2S,3R,4S,4aR)-2,3,4-trihydroxy-7-[(2- naphthylmethoxy]-(1,3)dioxolo(4,5-j)- 3,4,4a,5-tetrahydrophenanthridin-6(2H)- one | 8 | H | OH | OH | OH | H | ═O | 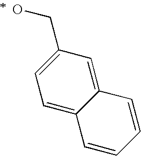 |
| (2S,3R,4S,4aR)-2,3,4-trihydroxy-7- [benzyloxy]-5-benzyl-(1,3)dioxolo(4,5-j)- 3,4,4a,5-tetrahydrophenanthridin-6(2H)- one | 9 | H | OH | OH | OH | 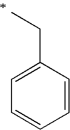 | ═O | 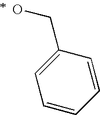 |
| (2S,3S,4S,4aR)-3,4-dihydroxy- | 10 | H | *OAc | OH | OH | H | ═O | *OAc |
| (1,3)dioxolo(4,5-j)-6-oxo-2,3,4,4a,5,6- | | | | | | | | |
| hexahydrophenanthridin--2,7-diyl | | | | | | | | |
| diacetate | | | | | | | | |
| (2S,3S,4S,4aR)-3,4,7-trihydroxy- | 11 | H | *OAc | OH | OH | H | ═O | OH |
| (1,3)dioxolo(4,5-j)-6-oxo-2,3,4,4a,5,6- | | | | | | | | |
| hexahydrophenanthridin--2-yl acetate | | | | | | | | |
| (2S,3S,4S,4aR)-7-hydroxyl-[2,2- dimethyl (1,3)dioxolo- (4,5-c)- (1,3)dioxolo(4,5-j)-6-oxo-2,3,4,4a,5,6- hexahydrophenanthridin-2-yl benzoate | 12 | H | *OBz | 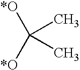 | | H | ═O | OH |
| (2S,3S,4S,4aR)-3,4,7-trihydroxy- | 13 | H | *OBz | OH | OH | H | ═O | OH |
| (1,3)dioxolo(4,5-j)-6-oxo-2,3,4,4a,5,6- | | | | | | | | |
| hexahydrophenanthridin--2-yl benzoate | | | | | | | | |
| (2S,3R,4S,4aR)-7-{[(2R,3R,4S,5R,6R)- | 14 | H | *OAc | *OAc | *OAc | H | ═O | Tetrabenzoyl-α-D- |
| 3,4,5-tribenzyyloxy-6- | | | | | | | | glucosyl* |
| (benzoyloxymethyl)tetrahydro-2H- | | | | | | | | |
| pyran-2-yl]oxy}-(1,3)dioxolo(4,5-j)-6- | | | | | | | | |
| oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-7-{[(2S,3R,4S,5R,6R)- | 15 | H | *OAc | *OAc | *OAc | H | ═O | Tetrabenzoyl-β-D- |
| 3,4,5-tribenzoyloxy-6- | | | | | | | | glucosyl* |
| (benzoyloxymethyl)tetrahydro-2H- | | | | | | | | |
| pyran-2-yl]oxy}--(1,3)dioxolo(4,5-j)-6- | | | | | | | | |
| oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-2,3,4-trihydroxy-7- | 16 | H | OH | OH | OH | H | ═O | α-D-glucosyl* |
| {[(2R,3R,4S,5S,6R)-3,4,5-trihydroxy-6- | | | | | | | | |
| (hydroxymethyl)tetrahydro-2H-pyran-2- | | | | | | | | |
| yl]oxy}-(1,3)dioxolo(4,5-j)-3,4,4a,5- | | | | | | | | |
| tetrahydrophenanthridine-6(2H)-one | | | | | | | | |
| (2S,3R,4S,4aR)-2,3,4-trihydroxy-7- | 17 | H | OH | OH | OH | H | ═O | β-D-glucosyl* |
| {[(2S,3R,4S,5S,6R)-3,4,5-trihydroxy-6- | | | | | | | | |
| (hydroxymethyl)tetrahydro-2H-pyran-2- | | | | | | | | |
| yl]oxy}-(1,3)dioxolo(4,5-j)-3,4,4a,5- | | | | | | | | |
| tetrahydrophenanthridine-6(2H)-one | | | | | | | | |
| (2S,3S,4S,4aR)-7-hydroxy-2,2-dimethyl (1,3)dioxolo-(4,5-c)-(1,3)dioxolo(4,5-j)- 6-oxo-2,3,4,4a,5,6- hexahydrophenanthridin-2-yl ethylcarbamate | 18 | H | 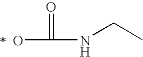 | 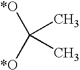 | | H | ═O | OH |
| (2S,3S,4S,4aR)-3,4,7-trihydroxy- (1,3)dioxolo(4,5-j)-6-oxo-2,3,4,4a,5,6- hexahydrophenanthridin-2-yl propionate | 19 | H | 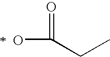 | OH | OH | H | ═O | OH |
| (2S,3S,4S,4aR)-3,4,7-trihydroxy- (1,3)dioxolo(4,5-j)-6-oxo-2,3,4,4a,5,6- hexahydrophenanthridin-2-yl 2- methylpropanoate | 20 | H | 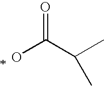 | OH | OH | H | ═O | OH |
| (2S,3S,4S,4aR)-2-(phenylcarbonyloxy)- | 21 | H | *OBz | OH | OH | H | ═O | OBz |
| 3,4-dihydroxy-(1,3)dioxolo(4,5-j)-6-oxo- | | | | | | | | |
| 2,3,4,4a,5,6-hexahydrophenanthridin-7- | | | | | | | | |
| yl benzoate | | | | | | | | |
| (2S,3S,4S,4aR)-2- {terbutyldimethylsilyloxy}-7-hydroxy- (1,3)dioxolo(4,5-j)-[2,2-dimethyl (1,3)dioxolo-(4,5-c)]-3,4,4a,5- tetrahydrophenanthridine-6(2H)-one | 22 | H | 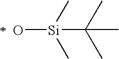 | 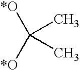 | | H | ═O | OH |
| (2S,3S,4S,4aR)-2-terbutyldimethylsilyl- (1,3)dioxolo(4,5-j)-[2,2-dimethyl (1,3)dioxolo-(4,5-c)]-2,3,4,4a,5,6- hexahydrophenanthridine-7-ol | 23 | H | 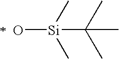 | 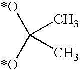 | | H | H | OH |
| (2S,3S,4S,4aR)-(1,3)dioxolo(4,5-j)-[2,2- dimethyl (1,3)dioxolo-(4,5-c)]- 2,3,4,4a,5,6-hexahydrophenanthridine- 2,7-diol | 24 | H | OH | 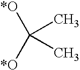 | | H | H | OH |
| (2S,3R,4S,4aR)-(1,3)dioxolo(4,5-j)- | 25 | H | OH | OH | OH | H | H | OH |
| 2,3,4,4a,5,6-hexahydrophenanthridine- | | | | | | | | |
| 2,3,4,7-tetrol | | | | | | | | |
| (2S,3R,4S,4aR)- 2(terbutyldimethylsilyloxy)-[2,2-dimethyl (1,3)dioxolo-(4,5-c)]-7-hydroxy- (1,3)dioxolo(4,5-j)-N-(trichloroacetyl)- 2,4,4a,6-tetrahydrophenanthridine- 5(3H)-carboxamide | 26 | H | 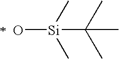 | 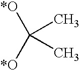 | | 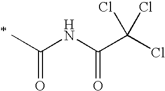 | H | OH |
| (2S,3R,4S,4aR)-2,3,4,7-tetrahydroxy- (1,3)dioxolo(4,5-j)-2,4,4a,6- tetrahydrophenanthridine-5(3H)- carboxamide | 27 | H | OH | OH | OH | 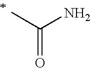 | H | OH |
| (2S,3R,4S,4aR)-5-acetyl- (1,3)dioxolo(4,5-j)-2,3,4,4a,5,6- hexahydrophenanthridine-2,3,4,7-tetrol | 28 | H | OH | OH | OH | 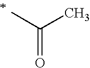 | H | OH |
| (2S,3R,4S,4aR)-5-methyl- | 29 | H | OH | OH | OH | Me | H | OH |
| (1,3)dioxolo(4,5-j)-2,3,4,4a,5,6- | | | | | | | | |
| hexahydrophenanthridine-2,3,4,7-tetrol | | | | | | | | |
| methyl (2S,3R,4S,4aR)-2,3,4,7- tetrahydroxy-6-oxo-(1,3)dioxolo(4,5-j)- 2,4,4a,6-tetrahydrophenanthridine- 5(3H)-carboxylate | 30 | H | OH | OH | OH | 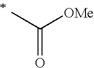 | ═O | OH |
| (2S,3R,4S,4aR)-2,3,7-trihydroxy-4- | 31 | H | *OH | *OH | *OCF₃ | H | ═O | *OH |
| (trifluoromethoxy)-(1,3)dioxolo(4,5-j)- | | | | | | | | |
| 3,4,4a,5-tetrahydrophenanthridin-6(2H)- | | | | | | | | |
| one | | | | | | | | |
| (2S,3R,4S,4aR)-4-(2- naphthylmethyloxy)-2,3,7-trihydroxy- (1,3)dioxolo(4,5-j)-3,4,4a,5- tetrahydrophenanthridin-6(2H)-one | 32 | H | *OH | *OH | 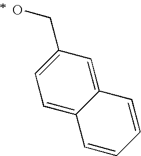 | H | ═O | *OH |
| methyl {(2S,3S,4S,4aS)-2,3-dihydroxy- 7-[4-methylbenzyloxy]-6-oxo- (1,3)dioxolo(4,5-j)-2,3,4,4a,5,6- hexahydrophenanthridin-4-yl}acetate | 33 | H | OH | OH | 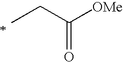 | H | ═O | 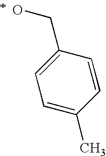 |
| [(2S,3S,4S,4aS)-2,3,7-trihydroxy-6-oxo- (1,3)dioxolo(4,5-j)-2,3,4,4a,5,6- hexahydrophenanthridin-4-yl]acetic acid | 34 | H | OH | OH | 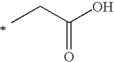 | H | ═O | *OH |
| (2S,3S,4S,4aS)-4-[2- (dimethylamino)ethyl]-2,3,7-trihydroxy-- (1,3)dioxolo(4,5-j)-3,4,4a,5- tetrahydrophenanthridin-6(2H)-one | 35 | H | OH | OH | 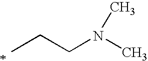 | H | ═O | *OH |
| (2S,3R,4S,4aR)-(1,3)dioxolo(4,5-j)-7- hydroxy-1-phenyl-6-oxo-2,3,4,4a,5,6- hexahydrophenanthridine-2,3,4-triyl triacetate | 36 | 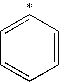 | *OAc | *OAc | *OAc | H | ═O | OH |
| (2S,3R,4S,4aR)-2,3,4,7-tetrahydroxy- (1,3)dioxolo(4,5-j)-1-phenyl-3,4,4a,5- tetrahydrophenanthridin-6(2H)-one | 37 | 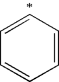 | OH | OH | OH | H | ═O | OH |
| (2S,3R,4S,4aR)-(1,3)dioxolo(4,5-j)-7- | 38 | *CH₃ | *OAc | *OAc | *OAc | H | ═O | OH |
| hydroxy-1-methyl-6-oxo-2,3,4,4a,5,6- | | | | | | | | |
| hexahydrophenanthridin--2,3,4-triyl | | | | | | | | |
| triacetate | | | | | | | | |
| (2S,3R,4S,4aR)-2,3,4,7-tetrahydroxy- | 39 | *CH₃ | OH | OH | OH | H | ═O | OH |
| (1,3)dioxolo(4,5-j)-1-methyl-3,4,4a,5- | | | | | | | | |
| tetrahydrophenanthridin-6(2H)-one | | | | | | | | |
| methyl {[(2S,3R,4S,4aR)-2,3,4- | 40 | H | *OAc | *OAc | *OAc | H | ═O | *OCH₂CO₂Me |
| tris[(methylcarbonyl)oxy]- | | | | | | | | |
| (1,3)dioxolo(4,5-j)-6-oxo-2,3,4,4a,5,6- | | | | | | | | |
| hexahydrophenanthridin-7- | | | | | | | | |
| yl]oxy}acetate | | | | | | | | |
| ethyl {[(2S,3R,4S,4aR)-2,3,4-trihydroxy- | 41 | H | OH | OH | OH | H | ═O | *OCH₂CO₂Et |
| (1,3)dioxolo(4,5-j)-6-oxo-2,3,4,4a,5,6- | | | | | | | | |
| hexahydrophenanthridin-7- | | | | | | | | |
| yl]oxy}acetate | | | | | | | | |
| ethyl {[(2S,3R,4S,4aR)-2,3,4- | 42 | H | *OAc | *OAc | *OAc | H | ═O | *OCH₂CO₂Et |
| tris[(methylcarbonyl)oxy]- | | | | | | | | |
| (1,3)dioxolo(4,5-j)-6-oxo-2,3,4,4a,5,6- | | | | | | | | |
| hexahydrophenanthridin--7- | | | | | | | | |
| yl]oxy}acetate | | | | | | | | |
| {[(2S,3R,4S,4aR)-2,3,4- | 43 | H | *OAc | *OAc | *OAc | H | ═O | *OCH₂CO₂H |
| tris[(methylcarbonyl)oxy]- | | | | | | | | |
| (1,3)dioxolo(4,5-j)-6-oxo-2,3,4,4a,5,6- | | | | | | | | |
| hexahydrophenanthridin-7-yl]oxy}acetic | | | | | | | | |
| acid | | | | | | | | |
| (2S,3R,4S,4aR)-7-(benzyloxy)-2,3,4- trihydroxy-(1,3)dioxolo(4,5-j)-3,4,4a,5- tetrahydrophenanthridin-6(2H)-one | 44 | H | OH | OH | OH | H | ═O | 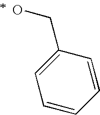 |
| (2S,3R,4S,4aR)-7-(benzyloxy)- (1,3)dioxolo(4,5-j)-6-oxo-2,3,4,4a,5,6- hexahydrophenanthridin-2,3,4-triyl triacetate | 45 | H | *OAc | *OAc | *OAc | H | ═O | 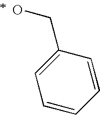 |
| (2S,3R,4S,4aR)-7-ethoxy-2,3,4- | 46 | H | OH | OH | OH | H | ═O | *OCH₂—CH₃ |
| trihydroxy-(1,3)dioxolo(4,5-j)-3,4,4a,5- | | | | | | | | |
| tetrahydrophenanthridin-6(2H)-one | | | | | | | | |
| (2S,3R,4S,4aR)-7-ethoxy- | 47 | H | *OAc | *OAc | *OAc | H | ═O | OCH₂—CH₃ |
| (1,3)dioxolo(4,5-j)--6-oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-2,3,4-trihydroxy- | 48 | H | OH | OH | OH | H | ═O | *OCH═CH₂ |
| (1,3)dioxolo(4,5-j)-7-(vinyloxy)-3,4,4a,5- | | | | | | | | |
| tetrahydrophenanthridin-6(2H)-one | | | | | | | | |
| (2S,3R,4S,4aR)-(1,3)dioxolo(4,5-j)-7- | 49 | H | *OAc | *OAc | *OAc | H | ═O | *OCH═CH₂ |
| (vinyloxy)-6-oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-7-[(3-fluorobenzyl)oxy]- 2,3,4-trihydroxy-(1,3)dioxolo(4,5-j)- 3,4,4a,5-tetrahydrophenanthridin-6(2H)- one | 50 | H | OH | OH | OH | H | ═O | 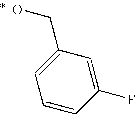 |
| (2S,3R,4S,4aR)-7-[(3-fluorobenzyl)oxy]- (1,3)dioxolo(4,5-j)-6-oxo-2,3,4,4a,5,6- hexahydrophenanthridin-2,3,4-triyl triacetate | 51 | H | *OAc | *OAc | *OAc | H | ═O | 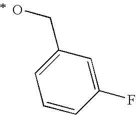 |
| (2S,3R,4S,4aR)-2,3,4-trihydroxy-7-[(4- nitrobenzyl)oxy]-(1,3)dioxolo(4,5-j)- 3,4,4a,5-tetrahydrophenanthridin-6(2H)- one | 52 | H | OH | OH | OH | H | ═O | 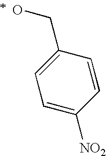 |
| (2S,3R,4S,4aR)-7-[(4-nitrobenzyl)oxy]- (1,3)dioxolo(4,5-j)-6-oxo-2,3,4,4a,5,6- hexahydrophenanthridin-2,3,4-triyl triacetate | 53 | H | *OAc | *OAc | *OAc | H | ═O | 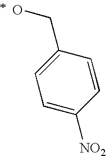 |
| (2S,3R,4S,4aR)-2,3,4-trihydroxy-7-[(4- methylbenzyl)oxy]-(1,3)dioxolo(4,5-j)- 3,4,4a,5-tetrahydrophenanthridin-6(2H)- one | 54 | H | OH | OH | OH | H | ═O | 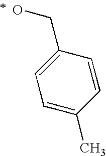 |
| (2S,3R,4S,4aR)-7-[(4- methylbenzyl)oxy]-(1,3)dioxolo(4,5-j)-- 6-oxo-2,3,4,4a,5,6- hexahydrophenanthridin-2,3,4-triyl triacetate | 55 | H | *OAc | *OAc | *OAc | H | ═O | 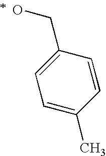 |
| (2S,3R,4S,4aR)-2,3,4-trihydroxy-7-[2- (2,5-dimethoxyphenyl)-2-oxoethoxy]- (1,3)dioxolo(4,5-j)-3,4,4a,5- tetrahydrophenanthridin-6(2H)-one | 56 | H | OH | OH | OH | H | ═O | 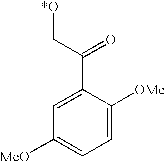 |
| (2S,3R,4S,4aR)-7-[2-(2,5- dimethoxyphenyl)-2-oxoethoxy]- (1,3)dioxolo(4,5-j)-6-oxo-2,3,4,4a,5,6- hexahydrophenanthridin-2,3,4-triyl triacetate | 57 | H | *OAc | *OAc | *OAc | H | ═O | 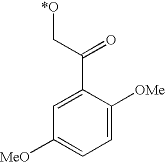 |
| (2S,3R,4S,4aR)-2,3,4-trihydroxy-7-[2- (3-nitrophenyl)-2-oxoethoxy]- (1,3)dioxolo(4,5-j)-3,4,4a,5- tetrahydrophenanthridin-6(2H)-one | 58 | H | OH | OH | OH | H | ═O | 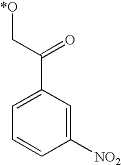 |
| (2S,3R,4S,4aR)-7-[2-(3-nitrophenyl)-2- oxoethoxy]-(1,3)dioxolo(4,5-j)-6-oxo- 2,3,4,4a,5,6-hexahydrophenanthridin- 2,3,4-triyl triacetate | 59 | H | *OAc | *OAc | *OAc | H | ═O | 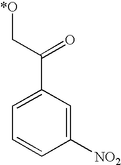 |
| sodium (2S,3R,4S,4aR)-2,3,4- | 60 | H | OH | OH | OH | H | ═O | *OSO₃Na |
| trihydroxy-(1,3)dioxolo(4,5-j)-6-oxo- | | | | | | | | |
| 2,3,4,4a,5,6-hexahydrophenanthridin-7- | | | | | | | | |
| yl sulfate | | | | | | | | |
| sodium (2S,3R,4S,4aR)-2,3,4- | 61 | H | *OAc | *OAc | *OAc | H | ═O | *OSO₃Na |
| tris[(methylcarbonyl)oxy]- | | | | | | | | |
| (1,3)dioxolo(4,5-j)-6-oxo-2,3,4,4a,5,6- | | | | | | | | |
| hexahydrophenanthridin-7-yl sulfate | | | | | | | | |
| pyridinium (2S,3R,4S,4aR)-2,3,4- trihydroxy-(1,3)dioxolo(4,5-j)-6-oxo- 2,3,4,4a,5,6-hexahydrophenanthridin-7- yl sulfate | 62 | H | OH | OH | OH | H | ═O | 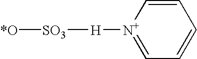 |
| pyridinium (2S,3R,4S,4aR)-2,3,4- tris[(methylcarbonyl)oxy]- (1,3)dioxolo(4,5-j)-6-oxo-2,3,4,4a,5,6- hexahydrophenanthridin-7-yl sulfate | 63 | H | *OAc | *OAc | *OAc | H | ═O | 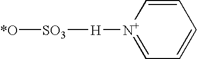 |
| (2S,3R,4S,4aR)-7-trifluoromethoxy- | 64 | H | OH | OH | OH | H | ═O | *OCF₃ |
| 2,3,4-trihydroxy-(1,3)dioxolo(4,5-j)- | | | | | | | | |
| 3,4,4a,5-tetrahydrophenanthridin-6(2H)- | | | | | | | | |
| one | | | | | | | | |
| (2S,3S,4S,4aR)-3,4,7-trihydroxy- (1,3)dioxolo(4,5-j)-6-oxo-2,3,4,4a,5,6- hexahydrophenanthridin-2-yl isonicotinate | 65 | H | 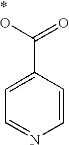 | OH | OH | H | ═O | OH |
| (2S,3S,4S,4aR)-3,4,7-trihydroxy- (1,3)dioxolo(4,5-j)-6-oxo-2,3,4,4a,5,6- hexahydrophenanthridin-2-yl morpholine-4-carboxylate | 66 | H | 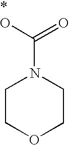 | OH | OH | H | ═O | OH |
| (2S,3S,4S,4aR)-3,4,7-trihydroxy- (1,3)dioxolo(4,5-j)-6-oxo-2,3,4,4a,5,6- hexahydrophenanthridin-2-yl cyclopentanecarboxylate | 67 | H | 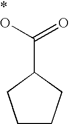 | OH | OH | H | ═O | OH |
| (2S,3S,4S,4aR)-7-(acetyloxy)-3 4- | 68 | H | *OCOEt | OH | OH | H | ═O | *OCOMe |
| dihydroxy-(1,3)dioxolo(4,5-j)-6-oxo- | | | | | | | | |
| 2,3,4,4a,5,6-hexahydrophenanthridin-2- | | | | | | | | |
| yl propionate | | | | | | | | |
| (2S,3S,4S,4aR)-2-(phenylcarbonyloxy)- [2,2-dimethyl (1,3)dioxolo-(4,5-c)]- (1,3)dioxolo(4,5-j)-6-oxo-2,3,4,4a,5,6- hexahydrophenanthridin-7-yl benzoate | 69 | H | *OBz | 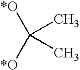 | | H | ═O | OBz |
| (2S,3R,4S,4aR)-2-amino-3,4,7- | 70 | H | *NH₂ | OH | OH | H | ═O | OH |
| trihydroxy-(1,3)dioxolo(4,5-j)-3,4,4a,5- | | | | | | | | |
| tetrahydrophenanthridin-6(2H)-one | | | | | | | | |
| (2S,3R,4S,4aR)-2-dimethylamino-3,4,7- | 71 | H | *N(CH₃)₂ | OH | OH | H | ═O | OH |
| trihydroxy-(1,3)dioxolo(4,5-j)-3,4,4a,5- | | | | | | | | |
| tetrahydrophenanthridin-6(2H)-one | | | | | | | | |
| (2S,3R,4S,4aR)-2-methylamino-3,4,7- | 72 | H | *NH(CH₃) | OH | OH | H | ═O | OH |
| trihydroxy-(1,3)dioxolo(4,5-j)-3,4,4a,5- | | | | | | | | |
| tetrahydrophenanthridin-6(2H)-one | | | | | | | | |
| (2S,3R,4S,4aR)-2-anilino-3,4,7- trihydroxy-(1,3)dioxolo(4,5-j)-3,4,4a,5- tetrahydrophenanthridin-6(2H)-one | 73 | H | 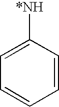 | OH | OH | H | ═O | OH |
| (2S,3R,4S,4aR)-2- [methyl(phenyl)amino]-3,4,7-trihydroxy- (1,3)dioxolo(4,5-j)-3,4,4a,5- tetrahydrophenanthridin-6(2H)-one | 74 | H | 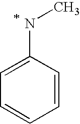 | OH | OH | H | ═O | OH |
| (2S,3R,4S,4aR)-2,3,7-trihydroxy- (1,3)dioxolo(4,5-j)-4-benzyloxy- 3,4,4a,5-tetrahydrophenanthridin-6(2H)- one | 75 | H | OH | OH | 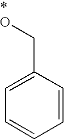 | H | ═O | OH |
| (2S,3R,4S,4aR)-2,3,7-trihydroxy- (1,3)dioxolo(4,5-j)-4-(4- fluoro)benzyloxy-3,4,4a,5- tetrahydrophenanthridin-6(2H)-one | 76 | H | OH | OH | 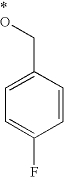 | H | ═O | OH |
| (2S,3R,4S,4aR)-2,3,7-trihydroxy- | 77 | H | OH | OH | *OMe | H | ═O | OH |
| (1,3)dioxolo(4,5-j)-4-methoxy-3,4,4a,5- | | | | | | | | |
| tetrahydrophenanthridin-6(2H)-one | | | | | | | | |
| (2S,3R,4S,4aR)-2,3,7-trihydroxy- | 78 | H | OH | OH | *OPr | H | ═O | OH |
| (1,3)dioxolo(4,5-j)-4-propoxy-3,4,4a,5- | | | | | | | | |
| tetrahydrophenanthridin-6(2H)-one | | | | | | | | |
| (2S,3R,4S,4aR)-2,3,4,7-tetrahydroxy- | 79 | H | OH | OH | OH | *CH₃ | ═O | OH |
| (1,3)dioxolo(4,5-j)-5-methyl-3,4,4a,5- | | | | | | | | |
| tetrahydrophenanthridin-6(2H)-one | | | | | | | | |
| (2S,3R,4S,4aR)-2,3,4,7-tetrahydroxy- | 80 | H | OH | OH | OH | *CH₂—CH₃ | ═O | OH |
| (1,3)dioxolo(4,5-j)-5-ethyl-3,4,4a,5- | | | | | | | | |
| tetrahydrophenanthridin-6(2H)-one | | | | | | | | |
| methyl [(2S,3R,4S,4aR)-2,3,4,7- | 81 | H | OH | OH | OH | *CH₂CO₂Me | ═O | OH |
| tetrahydroxy-(1,3)dioxolo(4,5-j)-6-oxo- | | | | | | | | |
| 2,4,4a,6-tetrahydrophenanthridin-5(3H)- | | | | | | | | |
| yl]acetate | | | | | | | | |
| methyl 3-[(2S,3R,4S,4aR)-2,3,4,7- | 82 | H | OH | OH | OH | *(CH₂)₂CO₂Me | ═O | OH |
| tetrahydroxy-(1,3)dioxolo(4,5-j)-6-oxo- | | | | | | | | |
| 2,4,4a,6-tetrahydrophenanthridin-5(3H)- | | | | | | | | |
| yl]propanoate | | | | | | | | |
| ethyl 4-[(2S,3R,4S,4aR)-2,3,4,7- | 83 | H | OH | OH | OH | *(CH₂)₃—CO₂Et | ═O | OH |
| tetrahydroxy-(1,3)dioxolo(4,5-j)-6-oxo- | | | | | | | | |
| 2,4,4a,6-tetrahydrophenanthridin-5(3H)- | | | | | | | | |
| yl]butanoate | | | | | | | | |
| (2S,3R,4S,4aR)-2,3,4,7-tetrahydroxy- | 84 | H | OH | OH | OH | H | ═S | OH |
| (1,3)dioxolo(4,5-j)-3,4,4a,5- | | | | | | | | |
| tetrahydrophenanthridine-6(2H)-thione | | | | | | | | |
| (2S,3S,4S,4aR)-2,3,4- | 85 | H | *OAc | *OAc | *OAc | H | ═S | *OAc |
| tris[(methylcarbonyl)oxy]- | | | | | | | | |
| (1,3)dioxolo(4,5-j)-6-thioxo-- | | | | | | | | |
| 2,3,4,4a,5,6-hexahydrophenanthridine- | | | | | | | | |
| 7-yl acetate | | | | | | | | |
| (2S,3R,4S,4aR)-7-hydroxy- | 86 | H | *OAc | *OAc | *OAc | H | ═S | OH |
| (1,3)dioxolo(4,5-j)-6-thioxo-- | | | | | | | | |
| 2,3,4,4a,5,6-hexahydrophenanthridine- | | | | | | | | |
| 2,3,4-triyl triacetate | | | | | | | | |
| (2S,3R,4S,4aR)-2,3,4-trihydroxy-7- methoxy-(1,3)dioxolo(4,5-j)-1-phenyl- 3,4,4a,5-hexahydrophenanthridin 6(2H)-one | 87 | 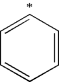 | OH | OH | OH | H | ═O | *OCH₃ |
| (2S,3R,4S,4aR)-(1,3)dioxolo(4,5-j)-7- methoxy-1-phenyl-6-oxo-2,3,4,4a,5,6- hexahydrophenanthridine-2,3,4-triyl triacetate | 88 | 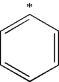 | *OAc | *OAc | *OAc | H | ═O | *OCH₃ |
| (2S,3R,4S,4aR)-(1,3)dioxolo(4,5-j)-7- | 89 | *CH₃ | *OAc | *OAc | *OAc | H | ═O | *OCH₃ |
| methoxy-1-methyl-6-oxo-2,3,4,4a,5,6- | | | | | | | | |
| hexahydrophenanthridine-2,3,4-triyl | | | | | | | | |
| triacetate | | | | | | | | |
| (2S,3R,4S,4aR)-2,3,4-trihydroxy-7- | 90 | *CH₃ | OH | OH | OH | H | ═O | *OCH₃ |
| methoxy-(1,3)dioxolo(4,5-j)-1-methyl- | | | | | | | | |
| 2,3,4,4a,5,6-hexahydrophenanthridin- | | | | | | | | |
| one | | | | | | | | |
| (2S,3S,4S,4aR)-3,4-dihydroxy-7- | 91 | H | *OAc | OH | OH | H | ═O | *OCH₃ |
| methoxy-(1,3)dioxolo(4,5-j)-6-oxo- | | | | | | | | |
| 2,3,4,4a,5,6-hexahydrophenanthridin-2- | | | | | | | | |
| yl acetate | | | | | | | | |
| (2S,3S,4S,4aR)-3,4-dihydroxy-7- | 92 | H | *OBz | OH | OH | H | ═O | *OCH₃ |
| methoxy-(1,3)dioxolo(4,5-j)-6-oxo- | | | | | | | | |
| 2,3,4,4a,5,6-hexahydrophenanthridin-2- | | | | | | | | |
| yl benzoate | | | | | | | | |
| (2S,3S,4S,4aR)-7-methoxy-[2,2- dimethyl (1,3)dioxolo-(4,5-c)]- (1,3)dioxolo(4,5-j)-6-oxo-2,3,4,4a,5,6- hexahydrophenanthridin-2-yl benzoate | 93 | H | *OBz | 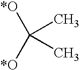 | | H | ═O | *OCH₃ |
| (2S,3R,4S,4aR)-2-amino-3,4-dihydroxy- | 94 | H | *NH₂ | OH | OH | H | ═O | *OCH₃ |
| 7-methyl-(1,3)dioxolo(4,5-j)-3,4,4a,5- | | | | | | | | |
| tetrahydrophenanthridin-6(2H)-one | | | | | | | | |
| (2S,3R,4S,4aR)-2-dimethylamino-3,4- | 95 | H | *N(CH₃)₂ | OH | OH | H | ═O | *OCH₃ |
| diydroxy-7-methoxy-(1,3)dioxolo(4,5-j)- | | | | | | | | |
| 3,4,4a,5-tetrahydrophenanthridin-6(2H)- | | | | | | | | |
| one | | | | | | | | |
| (2S,3R,4S,4aR)-2-methylamino-3,4- | 96 | H | *NH(CH₃) | OH | OH | H | ═O | *OCH₃ |
| dihydroxy-7-methoxy-(1,3)dioxolo(4,5- | | | | | | | | |
| j)-3,4,4a,5-tetrahydrophenanthridin- | | | | | | | | |
| 6(2H)-one | | | | | | | | |
| (2S,3R,4S,4aR)-2-anilino-3,4- dihydroxy-7-methoxy-(1,3)dioxolo(4,5- j)-3,4,4a,5-tetrahydrophenanthridin- 6(2H)-one | 97 | H | 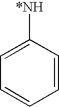 | OH | OH | H | ═O | *OCH₃ |
| (2S,3R,4S,4aR)-2- [methyl(phenyl)amino]-3,4-dihydroxy-7- methoxy-(1,3)dioxolo(4,5-j)-3,4,4a,5- tetrahydrophenanthridin-6(2H)-one | 98 | H | 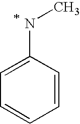 | OH | OH | H | ═O | *OCH₃ |
| (2S,3R,4S,4aR)-2,3-dihydroxy-7- | 99 | H | OH | OH | *OSO₃Na | H | ═O | *OCH₃ |
| methoxy-(1,3)dioxolo(4,5-j)-6-oxo- | | | | | | | | |
| 2,3,4,4a,5,6-hexahydrophenanthridin-4- | | | | | | | | |
| yl sodium sulfate | | | | | | | | |
| (2S,3R,4S,4aR)-2,3-dihydroxy-7- methoxy-(1,3)dioxolo(4,5-j)-4- benzyloxy-3,4,4a,5- tetrahydrophenanthridin-6(2H)-one | 100 | H | OH | OH | 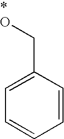 | H | ═O | *OCH₃ |
| (2S,3R,4S,4aR)-2,3-dihydroxy-7- methoxy-(1,3)dioxolo(4,5-j)-4-(4- fluoro)benzyloxy-3,4,4a,5- tetrahydrophenanthridin-6(2H)-one | 101 | H | OH | OH | 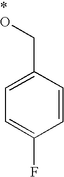 | H | ═O | *OCH₃ |
| (2S,3R,4S,4aR)-2,3-dihydroxy-7- | 102 | H | OH | OH | *OMe | H | ═O | *OCH₃ |
| methoxy-(1,3)dioxolo(4,5-j)-4-methoxy- | | | | | | | | |
| 3,4,4a,5-tetrahydrophenanthridin-6(2H)- | | | | | | | | |
| one | | | | | | | | |
| (2S,3R,4S,4aR)--2,3-dihydroxy-7- | 103 | H | OH | OH | *OPr | H | ═O | *OCH₃ |
| methoxy-(1,3)dioxolo(4,5-j)-4-propoxy- | | | | | | | | |
| 3,4,4a,5-tetrahyd rophenanthridin-6(2H)- | | | | | | | | |
| one | | | | | | | | |
| (2S,3R,4S,4aR)-2,3,4-trihydroxy-7- | 104 | H | OH | OH | OH | *CH₃ | ═O | *OCH₃ |
| methoxy-(1,3)dioxolo(4,5-j)-5-methyl- | | | | | | | | |
| 3,4,4a,5-tetrahydrophenanthridin-6(2H)- | | | | | | | | |
| one | | | | | | | | |
| (2S,3R,4S,4aR)-2,3,4-trihydroxy-7- | 105 | H | OH | OH | OH | *CH₂—CH₃ | ═O | *OCH₃ |
| methoxy-(1,3)dioxolo(4,5-j)-5-ethyl- | | | | | | | | |
| 3,4,4a,5-tetrahydrophenanthridin-6(2H)- | | | | | | | | |
| one | | | | | | | | |
| methyl [(2S,3R,4S,4aR)-2,3,4- | 106 | H | OH | OH | OH | *CH₂—CO₂Me | ═O | *OCH₃ |
| trihydroxy-7-methoxy-(1,3)dioxolo(4,5- | | | | | | | | |
| j)-6-oxo-2,4,4a,6- | | | | | | | | |
| tetrahydrophenanthridin-5(3H)- | | | | | | | | |
| yl]acetate | | | | | | | | |
| methyl 3-[(2S,3R,4S,4aR)-2,3,4- | 107 | H | OH | OH | OH | *(CH₂)₂CO₂Me | ═O | *OCH₃ |
| trihydroxy-7-methoxy-(1,3)dioxolo(4,5- | | | | | | | | |
| j)-6-oxo-2,4,4a,6- | | | | | | | | |
| tetrahydrophenanthridin-5(3H)- | | | | | | | | |
| yl]propanoate | | | | | | | | |
| ethyl 4-[(2S,3R,4S,4aR)-2,3,4- | 108 | H | OH | OH | OH | *(CH₂)₃—CO₂Et | ═O | *OCH₃ |
| trihydroxy-7-methoxy-(1,3)dioxolo(4,5- | | | | | | | | |
| j)-6-oxo-2,4,4a,6- | | | | | | | | |
| tetrahydrophenanthridin-5(3H)- | | | | | | | | |
| yl]butanoate | | | | | | | | |
| (2S,3R,4S,4aR)-2,3,4-trihydroxy-7- | 109 | H | OH | OH | OH | H | ═S | *OCH₃ |
| methoxy-(1,3)dioxolo(4,5-j)-3,4,4a,5- | | | | | | | | |
| tetrahydrophenanthridine-6(2H)-thione | | | | | | | | |
| (2S,3R,4S,4aR)-3,4- | 110 | H | *OAc | *OAc | *OAc | H | ═S | *OCH₃ |
| di[(methylcarbonyl)oxy]-7-methoxy- | | | | | | | | |
| (1,3)dioxolo(4,5-j)-6-thioxo-2,3,4,4a,5,6- | | | | | | | | |
| hexahydrophenanthridin-2-yl acetate | | | | | | | | |
| (2S,3R,4S,4aR)-7-{[(2R,3R,4R,5S,6S)- | 111 | H | *OAc | *OAc | *OAc | H | ═O | tribenzoyl β-L- |
| 3,4,5-tribenzoyloxy-6-methyltetrahydro- | | | | | | | | Rhamnosyl |
| 2H-pyran-2-yl]oxy}-(1,3)dioxolo(4,5-j)-6- | | | | | | | | |
| oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-2,3,4-trihydroxy-7- | 112 | H | OH | OH | OH | H | ═O | β-L-Rhamnosyl |
| {[(2R,3R,4R,5R,6S)-3,4,5-trihydroxy-6- | | | | | | | | |
| methyltetrahydro-2H-pyran-2-yl]oxy}- | | | | | | | | |
| (1,3)dioxolo(4,5-j)-3,4,4a,5- | | | | | | | | |
| tetrahydrophenanthridine-6(2H)-one | | | | | | | | |
| (2S,3R,4S,4aR)-7-{[(2R,3R,4S,5S,6R)- | 113 | H | *OAc | *OAc | *OAc | H | ═O | Tetrabenzoyl β-D- |
| 3,4,5-tribenzyloxy-6- | | | | | | | | Mannosyl |
| (benzoyloxymethyl)tetrahydro-2H- | | | | | | | | |
| pyran-2-yl]oxy}-(1,3)dioxolo(4,5-j)-6- | | | | | | | | |
| oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-2,3,4-trihydroxy-7-7- | 114 | H | OH | OH | OH | H | ═O | β-D-Mannosyl |
| {[(2S,3S,4S,5S,6R)-3,4,5-trihydroxy-6- | | | | | | | | |
| (hydroxymethyl)tetrahydro-2H-pyran-2- | | | | | | | | |
| yl]oxy}-(1,3)dioxolo(4,5-j)-3,4,4a,5- | | | | | | | | |
| tetrahydrophenanthridine-6(2H)-one | | | | | | | | |
| (2S,3R,4S,4aR)-7-7- | 115 | H | *OAc | *OAc | *OAc | H | ═O | tribenzoyl β-L- |
| {[(2R,3S,4R,5R,6S)-3,4,5- | | | | | | | | Fucosyl |
| tribenzoyloxy-6-methyltetrahydro-2H- | | | | | | | | |
| pyran-2-yl]oxy}-(1,3)dioxolo(4,5-j)-6- | | | | | | | | |
| oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-2,3,4-trihydroxy-7- | 116 | H | OH | OH | OH | H | ═O | β-L-Fucosyl |
| {[(2R,3R,4R,5S,6S)-3,4,5-trihydroxy-6- | | | | | | | | |
| methyltetrahydro-2H-pyran-2-yl]oxy}- | | | | | | | | |
| (1,3)dioxolo(4,5-j)-3,4,4a,5- | | | | | | | | |
| tetrahydrophenanthridine-6(2H)-one | | | | | | | | |
| (2S,3R,4S,4aR)-7-{[(2S,3S,4S,5R,6R)- | 117 | H | *OAc | *OAc | *OAc | H | ═O | heptabenzoyl β-D- |
| 3,4,5-tribenzoyloxy-6- | | | | | | | | Isomaltosyl |
| ({[(2S,3R,4S,5R,6R)-3,4,5- | | | | | | | | |
| tribenzoyloxy-6- | | | | | | | | |
| (benzoyloxymethyl)tetrahydro-2H- | | | | | | | | |
| pyran-2-yl]oxy}methyl)tetrahydro-2H- | | | | | | | | |
| pyran-2-yl]oxy}-(1,3)dioxolo(4,5-j)-6- | | | | | | | | |
| oxo-2,3,4,4a,5,6- | | | | | | | | |
| hexahydrophenanthridin-2,3,4-triyl | | | | | | | | |
| triacetate | | | | | | | | |
| (2S,3R,4S,4aR)-2,3,4-trihydroxy-7- | 118 | H | OH | OH | OH | H | ═O | β-D-Isomaltosyl |
| {[(2R,3S,4S,5R,6S)-3,4,5-trihydroxy-6- | | | | | | | | |
| ({[(2R,3S,4S,5R,6S)-3,4,5-trihydroxy-6- | | | | | | | | |
| (hydroxymethyl)tetrahydro-2H-pyran-2- | | | | | | | | |
| yl]oxy}methyl)tetrahydro-2H-pyran-2- | | | | | | | | |
| yl]oxy}-(1,3)dioxolo(4,5-j)-3,4,4a,5- | | | | | | | | |
| tetrahydrophenanthridine-6(2H)-one | | | | | | | | |
| (2S,3S,4S,4aR)-2,7- bis{[(2S,3R,4S,5R,6R)-3,4,5- tribenzoyloxy-6- (benzoyloxymethyl)tetrahydro-2H- pyran-2-yl]oxy}-(1,3)dioxolo(4,5-j)-[2,2- dimethyl (1,3)dioxolo-(4,5-c)]-3,4,4a,5- tetrahydrophenanthridine-6(2H)-one | 119 | H | Tetrabenzoyl O-β-D-Glucosyl | 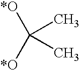 | | H | ═O | Tetrabenzoyl β-D-Glucosyl |
| (2S,3S,4S,4aR)-2,7- | 120 | H | Tetrabenzoyl | OH | OH | H | ═O | Tetrabenzoyl |
| bis{[(2S,3R,4S,5R,6R)-3,4,5- | | | O-β-D-Glucosyl | | | | | β-D-Glucosyl |
| tribenzoyloxy-6- | | | | | | | | |
| (benzoyloxymethyl)tetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)-one | | | | | | | | |
| (2S,3S,4S,4aR)-2,7- | 121 | H | O-β-D-Glucosyl | OH | OH | H | ═O | β-D-Glucosyl |
| bis{[(2S,3R,4S,5S,6R)-3,4,5-trihydroxy- | | | | | | | | |
| 6-(hydroxymethyl)tetrahydro-2H-pyran- | | | | | | | | |
| 2-yl]oxy}-3,4-dihydroxy- | | | | | | | | |
| (1,3)dioxolo(4,5-j)-3,4,4a,5- | | | | | | | | |
| tetrahydrophenanthridine-6(2H)-one | | | | | | | | |
| (2S,3S,4S,4aR)-2,7- bis{[(2R,3R,4R,5S,6S)-3,4,5- tribenzoyloxy-6-methyltetrahydro-2H- pyran-2-yl]oxy}-(1,3)dioxolo(4,5-j)-[2,2- dimethyl (1,3)dioxolo-(4,5-c)]-3,4,4a,5- tetrahydrophenanthridine-6(2H)-one | 122 | H | Tribenzoyl O-β-L- Rhamnosyl | 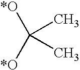 | | H | ═O | Tribenzoyl β-L- Rhamnosyl |
| (2S,3S,4S,4aR)-2,7- | 123 | H | Tribenzoyl O-β-L- | OH | OH | H | ═O | Tribenzoyl β-L- |
| bis{[(2R,3R,4R,5S,6S)-3,4,5- | | | Rhamnosyl | | | | | Rhamnosyl |
| tribenzoyloxy-6-methyltetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)-one | | | | | | | | |
| (2S,3S,4S,4aR)-2,7- | 124 | H | O-β-L-Rhamnosyl | OH | OH | H | ═O | β-L-Rhamnosyl |
| bis{[(2R,3R,4R,5R,6S)-3,4,5-trihydroxy- | | | | | | | | |
| 6-methyltetrahydro-2H-pyran-2-yl]oxy}- | | | | | | | | |
| 3,4-dihydroxy-(1,3)dioxolo(4,5-j)- | | | | | | | | |
| 3,4,4a,5-tetrahydrophenanthridine- | | | | | | | | |
| 6(2H)-one | | | | | | | | |
| (2S,3S,4S,4aR)-2,7- bis{[(2R,3R,4S,5S,6R)-3,4,5- tribenzyloxy-6- (benzoyloxymethyl)tetrahydro-2H- pyran-2-yl]oxy}-(1,3)dioxolo(4,5-j)-[2,2- dimethyl (1,3)dioxolo-(4,5-c)]-3,4,4a,5- tetrahydrophenanthridine-6(2H)-one | 125 | H | Tetrabenzoyl O-β-D- Mannosyl | 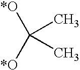 | | H | ═O | Tetrabenzoyl β-D-Mannosyl |
| (2S,3S,4S,4aR)-2,7- | 126 | H | Tetrabenzoyl O-β-D- | OH | OH | H | ═O | Tetrabenzoyl β-D- |
| bis{[(2R,3R,4S,5S,6R)-3,4,5- | | | Mannosyl | | | | | Mannosyl |
| tribenzyloxy-6- | | | | | | | | |
| (benzoyloxymethyl)tetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)-one | | | | | | | | |
| (2S,3S,4S,4aR)-2,7- | 127 | H | O-β-D-Mannosyl | OH | OH | H | ═O | β-D-Mannosyl |
| bis{[(2S,3S,4S,5S,6R)-3,4,5-trihydroxy- | | | | | | | | |
| 6-(hydroxymethyl)tetrahydro-2H-pyran- | | | | | | | | |
| 2-yl]oxy}-3,4-dihydroxy- | | | | | | | | |
| (1,3)dioxolo(4,5-j)-3,4,4a,5- | | | | | | | | |
| tetrahydrophenanthridine-6(2H)-one | | | | | | | | |
| (2S,3S,4S,4aR)-2,7- bis{[(2R,3S,4R,5R,6S)-3,4,5- tribenzoyloxy-6-methyltetrahydro-2H- pyran-2-yl]oxy}-(1,3)dioxolo(4,5-j)-[2,2- dimethyl (1,3)dioxolo-(4,5-c)]-3,4,4a,5- tetrahydrophenanthridine-6(2H)-one | 128 | H | Tribenzoyl O-β-L- Fucosyl | 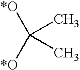 | | H | ═O | Tribenzoyl β-L- Fucosyl |
| (2S,3S,4S,4aR)-2,7- | 129 | H | Tribenzoyl O-β-L- | OH | OH | H | ═O | Tribenzoyl β-L- |
| bis{[(2R,3S,4R,5R,6S)-3,4,5- | | | Fucosyl | | | | | Fucosyl |
| tribenzoyloxy-6-methyltetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)-one | | | | | | | | |
| (2S,3S,4S,4aR)-2,7- | 130 | H | O-β-L-Fucosyl | OH | OH | H | ═O | β-L-Fucosyl |
| bis{[(2R,3R,4R,5S,6S)-3,4,5-trihydroxy- | | | | | | | | |
| 6-methyltetrahydro-2H-pyran-2-yl]oxy}- | | | | | | | | |
| 3,4-dihydroxy-(1,3)dioxolo(4,5-j)- | | | | | | | | |
| 3,4,4a,5-tetrahydrophenanthridine- | | | | | | | | |
| 6(2H)-one | | | | | | | | |
| (2S,3S,4S,4aR)-2,7- bis{[(2S,3S,4S,5R,6R)-3,4,5- tribenzoyloxy-6-({[(2S,3R,4S,5R,6R)- 3,4,5-tribenzoyloxy-6- (benzoyloxymethyl)tetrahydo-2H- pyran-2-yl]oxy}methyl)tetrahydro-2H- pyran-2-yl]oxy}-(1,3)dioxolo(4,5-j)-[2,2- dimethyl (1,3)dioxolo-(4,5-c)]-3,4,4a,5- tetrahydrophenanthridine-6(2H)-one | 131 | H | heptabenzoyl O-β-D- Isomaltosyl | 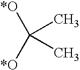 | | H | ═O | heptabenzoyl β-D- Isomaltosyl |
| (2S,3S,4S,4aR)-2,7- | 132 | H | heptabenzoyl O-β-D- | OH | OH | H | ═O | heptabenzoyl β-D- |
| bis{[(2S,3S,4S,5R,6R)-3,4,5- | | | Isomaltosyl | | | | | Isomaltosyl |
| tribenzoyloxy-6-({[(2S,3R,4S,5R,6R)- | | | | | | | | |
| 3,4,5-tribenzoyloxy-6- | | | | | | | | |
| (benzoyloxymethyl)tetrahydro-2H- | | | | | | | | |
| pyran-2-yl]oxy}methyl)tetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)-one | | | | | | | | |
| (2S,3S,4S,4aR)-2,7- | 133 | H | O-β-D-Isomaltosyl | OH | OH | H | ═O | β-D-Isomaltosyl |
| bis{[(2R,3S,4S,5R,6S)-3,4,5-trihydroxy- | | | | | | | | |
| 6-({[(2R,3S,4S,5R,6S)-3,4,5-trihydroxy- | | | | | | | | |
| 6-(hydroxymethyl)tetrahydro-2H-pyran- | | | | | | | | |
| 2-yl]oxy}methyl)tetrahydro-2H-pyran-2- | | | | | | | | |
| yl]oxy}-3,4-dihydroxy-(1,3)dioxolo(4,5- | | | | | | | | |
| j)-3,4,4a,5-tetrahydrophenanthridine- | | | | | | | | |
| 6(2H)-one | | | | | | | | |
| (2S,3S,4S,4aR)-2,7-bis{N- methylaminocarbonyloxy}-3,4- dihydroxy-(1,3)dioxolo(4,5-j)-3,4,4a,5- tetrahydrophenanthridine-6(2H)-one | 134 | H | 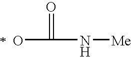 | OH | OH | H | ═O | 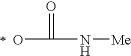 |
| (2S,3S,4S,4aR)-2-{N- methylaminocarbonyloxy}-3,4,7- trihydroxy-(1,3)dioxolo(4,5-j)-3,4,4a,5- tetrahydrophenanthridine-6(2H)-one | 135 | H | 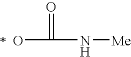 | OH | OH | H | ═O | OH |
| (2S,3S,4S,4aR)-2,7-bis{N- methylaminocarbonyloxy}- (1,3)dioxolo(4,5-j)-[2,2-dimethyl (1,3)dioxolo-(4,5-c)]-3,4,4a,5- tetrahydrophenanthridine-6(2H)-one | 136 | H | 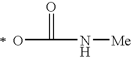 | 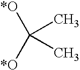 | | H | ═O | 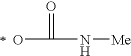 |
| (2S,3S,4S,4aR)-2,7-bis(N- ethylaminocarbonyloxy)-3,4-dihydroxy- (1,3)dioxolo(4,5-j)-3,4,4a,5- tetrahydrophenanthridine-6(2H)-one | 137 | H | 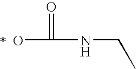 | OH | OH | H | ═O | 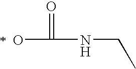 |
| (2S,3S,4S,4aR)-2-{N- ethylaminocarbonyloxy}-3,4,7- trihydroxy-(1,3)dioxolo(4,5-j)-3,4,4a,5- tetrahydrophenanthridine-6(2H)-one | 138 | H | 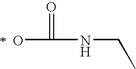 | OH | OH | H | ═O | OH |
| (2S,3S,4S,4aR)-2,7-bis{N- ethylaminocarbonyloxy}- (1,3)dioxolo(4,5-j)-[2,2-dimethyl (1,3)dioxolo-(4,5-c)]-3,4,4a,5- tetrahydrophenanthridine-6(2H)-one | 139 | H | 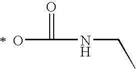 | 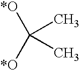 | | H | ═O | 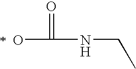 |
| (2S,3S,4S,4aR)-2,7-bis(N- isopropylaminocarbonyloxy)-3,4- dihydroxy-(1,3)dioxolo(4,5-j)-3,4,4a,5- tetrahydrophenanthridine-6(2H)-one | 140 | H | 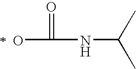 | OH | OH | H | ═O | 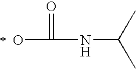 |
| (2S,3S,4S,4aR)-2,7-bis{N- isopropylaminocarbonyloxy}- (1,3)dioxolo(4,5-j)-[2,2-dimethyl (1,3)dioxolo-(4,5-c)]-3,4,4a,5- tetrahydrophenanthridine-6(2H)-one | 141 | H | 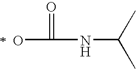 | 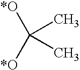 | | H | ═O | 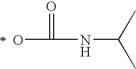 |
| (2S,3S,4S,4aR)-2-{N- isopropylaminocarbonyloxy}-3,4,7- trihydroxy-(1,3)dioxolo(4,5-j)-3,4,4a,5- tetrahydrophenanthridine-6(2H)-one | 142 | H | 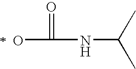 | OH | OH | H | ═O | OH |
| (2S,3S,4S,4aR)-2,7-bis(N- phenylaminocarbonyloxy)-3,4- dihydroxy-(1,3)dioxolo(4,5-j)-3,4,4a,5- tetrahydrophenanthridine-6(2H)-one | 143 | H | 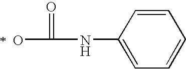 | OH | OH | H | ═O | 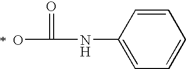 |
| (2S,3S,4S,4aR)-2,7-bis{N- phenylaminocarbonyloxy}- (1,3)dioxolo(4,5-j)-[2,2-dimethyl (1,3)dioxolo-(4,5-c)]-3,4,4a,5- tetrahydrophenanthridine-6(2H)-one | 144 | H | 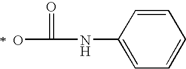 | 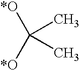 | | H | ═O | 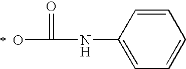 |
| (2S,3S,4S,4aR)-2-{N- phenylaminocarbonyloxy}-3,4,7- trihydroxy-(1,3)dioxolo(4,5-j)-3,4,4a,5- tetrahydrophenanthridine-6(2H)-one | 145 | H | 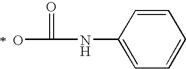 | OH | OH | H | ═O | OH |
| (2S,3S,4S,4aR)-2,7- bis(ethylcarbonyloxy)-3,4-dihydroxy- (1,3)dioxolo(4,5-j)-3,4,4a,5- tetrahydrophenanthridine-6(2H)-one | 146 | H | 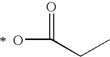 | OH | OH | H | ═O | 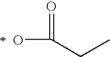 |
| (2S,3S,4S,4aR)-2,7- bis{ethylcarbonyloxy}-(1,3)dioxolo(4,5- j)-[2,2-dimethyl (1,3)dioxolo-(4,5-c)]- 3,4,4a,5-tetrahydrophenanthridine- 6(2H)-one | 147 | H | 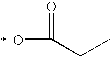 | 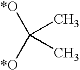 | | H | ═O | 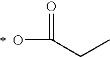 |
| (2S,3S,4S,4aR)-2-{N- ethylcarbonyloxy}-3,4,7-trihydroxy- (1,3)dioxolo(4,5-j)-3,4,4a,5- tetrahydrophenanthridine-6(2H)-one | 148 | H | 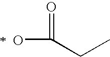 | OH | OH | H | ═O | OH |
| (2S,3S,4S,4aR)-2,7- bis(isopropylcarbonyloxy)-3,4- dihydroxy-(1,3)dioxolo(4,5-j)-3,4,4a,5- tetrahydrophenanthridine-6(2H)-one | 149 | H | 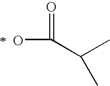 | OH | OH | H | ═O | 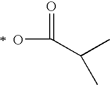 |
| (2S,3S,4S,4aR)-2,7- bis{isopropylcarbonyloxy}- (1,3)dioxolo(4,5-j)-[2,2-dimethyl (1,3)dioxolo-(4,5-c)]-3,4,4a,5- tetrahydrophenanthridine-6(2H)-one | 150 | H | 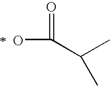 | 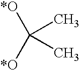 | | H | ═O | 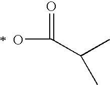 |
| (2S,3S,4S,4aR)-2- {isopropylcarbonyloxy}-3,4,7-trihydroxy- (1,3)dioxolo(4,5-j)-3,4,4a,5- tetrahydrophenanthridine-6(2H)-one | 151 | H | 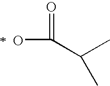 | OH | OH | H | ═O | OH |
| (2S,3S,4S,4aR)-2,7-bis(prop-1-en- carbonyloxy)-3,4-dihydroxy- (1,3)dioxolo(4,5-j)-3,4,4a,5- tetrahydrophenanthridine-6(2H)-one | 152 | H | 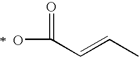 | OH | OH | H | ═O | 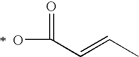 |
| (2S,3S,4S,4aR)-2,7-bis{prop-1-en- carbonyloxy}-(1,3)dioxolo(4,5-j)-[2,2- dimethyl (1,3)dioxolo-(4,5-c)]-3,4,4a,5- tetrahydrophenanthridine-6(2H)-one | 153 | H | 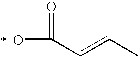 | 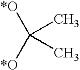 | | H | ═O | 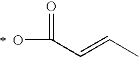 |
| (2S,3S,4S,4aR)-2-{prop-1-en- carbonyloxy}-3,4,7-trihydroxy- (1,3)dioxolo(4,5-j)-3,4,4a,5- tetrahydrophenanthridine-6(2H)-one | 154 | H | 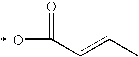 | OH | OH | H | ═O | OH |
| (2S,3S,4S,4aR)-2,7-bis(2- methylpropan-2-carbonyloxy)-3,4- dihydroxy-(1,3)dioxolo(4,5-j)-3,4,4a,5- tetrahydrophenanthridine-6(2H)-one | 155 | H | 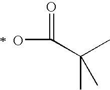 | OH | OH | H | ═O | 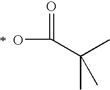 |
| (2S,3S,4S,4aR)-2,7-bis{2- methylpropan-2-carbonyloxy}- (1,3)dioxolo(4,5-j)-[2,2-dimethyl (1,3)dioxolo-(4,5-c)]-3,4,4a,5- tetrahydrophenanthridine-6(2H)-one | 156 | H | 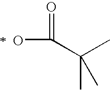 | 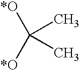 | | | | 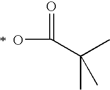 |
| (2S,3S,4S,4aR)-2-{2-methylpropan-2- carbonyloxy}-3,4,7-trihydroxy- (1,3)dioxolo(4,5-j)-3,4,4a,5- tetrahydrophenanthridine-6(2H)-one | 157 | H | 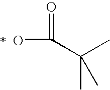 | OH | OH | H | ═O | OH |
| (2S,3S,4S,4aR)-2,7-bis(N- methylaminothiocarbonyloxy)-3,4- dihydroxy-(1,3)dioxolo(4,5-j)-3,4,4a,5- tetrahydrophenanthridine-6(2H)-one | 158 | H | 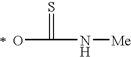 | OH | OH | H | ═O | 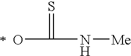 |
| (2S,3S,4S,4aR)-2-{N- methylaminothiocarbonyloxy}-3,4,7- trihydroxy-(1,3)dioxolo(4,5-j)-3,4,4a,5- tetrahydrophenanthridine-6(2H)-one | 159 | H | 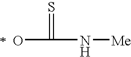 | OH | OH | H | ═O | OH |
| (2S,3S,4S,4aR)-2,7-bis{N- methylaminothiocarbonyloxy}- (1,3)dioxolo(4,5-j)-[2,2-dimethyl (1,3)dioxolo-(4,5-c)]-3,4,4a,5- tetrahydrophenanthridine-6(2H)-one | 160 | H | 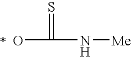 | 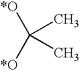 | | H | ═O | 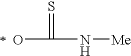 |
| (2S,3S,4S,4aR)-2,7-bis(N- ethylaminothiocarbonyloxy)-3,4- dihydroxy-(1,3)dioxolo(4,5-j)-3,4,4a,5- tetrahydrophenanthridine-6(2H)-one | 161 | H | 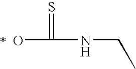 | OH | OH | H | ═O | 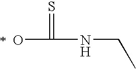 |
| (2S,3S,4S,4aR)-2-{N- ethylaminothiocarbonyloxy}-3,4,7- trihydroxy-(1,3)dioxolo(4,5-j)-3,4,4a,5- tetrahydrophenanthridine-6(2H)-one | 162 | H | 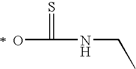 | OH | OH | H | ═O | OH |

| | | | | | | | |
|---|---|---|---|---|---|---|---|
| (2S,3S,4S,4aR)-2,7-bis{N- ethylaminothiocarbonyloxy}- (1,3)dioxolo(4,5-j)-[2,2-dimethyl (1,3)dioxolo-(4,5-c)]-3,4,4a,5- tetrahydrophenanthridine-6(2H)-one | 163 | H | 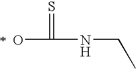 | 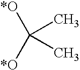 | H | ═O | 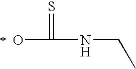 |

TABLE 2

| | |
|---|---|
| | IV |
| 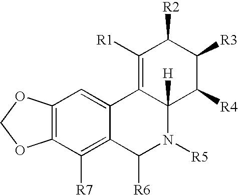 | |

| Name | Compound | R¹ | R² | R³ | R⁴ | R⁵ | R⁶ | R⁷ |
|---|---|---|---|---|---|---|---|---|
| (2R,3R,4S,4aR)-2-amino-3,4,7- | 164 | H | *NH₂ | OH | OH | H | ═O | OH |
| trihydroxy-(1,3)dioxolo(4,5-j)- | | | | | | | | |
| 3,4,4a,5- | | | | | | | | |
| tetrahydrophenanthridin-6(2H)- | | | | | | | | |
| one | | | | | | | | |
| (2R,3R,4S,4aR)-2- | 165 | H | *N(CH₃)₂ | OH | OH | H | ═O | OH |
| dimethylamino-3,4,7-trihydroxy- | | | | | | | | |
| (1,3)dioxolo(4,5-j)-3,4,4a,5- | | | | | | | | |
| tetrahydrophenanthridin-6(2H)- | | | | | | | | |
| one | | | | | | | | |
| (2R,3R,4S,4aR)-2-methylamino- | 166 | H | *NH(CH₃) | OH | OH | H | ═O | OH |
| 3,4,7-trihydroxy- | | | | | | | | |
| (1,3)dioxolo(4,5-j)-3,4,4a,5- | | | | | | | | |
| tetrahydrophenanthridin-6(2H)- | | | | | | | | |
| one | | | | | | | | |
| (2R,3R,4S,4aR)-2-anilino-3,4,7- trihydroxy-(1,3)dioxolo(4,5-j)- 3,4,4a,5- tetrahydrophenanthridin-6(2H)- one | 167 | H | 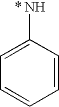 | OH | OH | H | ═O | OH |
| (2R,3R,4S,4aR)-2- [methyl(phenyl)amino]-3,4,7- trihydroxy-(1,3)dioxolo(4,5-j)- 3,4,4a,5- tetrahydrophenanthridin-6(2H)- one | 168 | H | 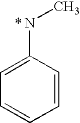 | OH | OH | H | ═O | OH |
| (2R,3S,4S,4aR)-7-methoxy- [2,2-dimethyl(1,3)dioxolo-(4,5- c)]-(1,3)dioxolo(4,5-j)-6-oxo- 3,4,4a,5- tetrahydrophenanthridin-2-yl benzoate | 169 | H | *OBz | 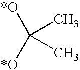 | | H | ═O | *OCH₃ |
| (2R,3R,4S,4aR)-2-amino-3,4- | 170 | H | *NH₂ | OH | OH | H | ═O | *OCH₃ |
| dihydroxy-7-methoxy- | | | | | | | | |
| (1,3)dioxolo(4,5-j)-3,4,4a,5- | | | | | | | | |
| tetrahydrophenanthridin-6(2H)- | | | | | | | | |
| one | | | | | | | | |
| (2R,3R,4S,4aR)-2- | 171 | H | *N(CH₃)₂ | OH | OH | H | ═O | *OCH₃ |
| dimethylamino-3,4-diydroxy-7- | | | | | | | | |
| methoxy-(1,3)dioxolo(4,5-j)- | | | | | | | | |
| 3,4,4a,5- | | | | | | | | |
| tetrahydrophenanthridin-6(2H)- | | | | | | | | |
| one | | | | | | | | |
| (2R,3R,4S,4aR)-2-methylamino- | 172 | H | *NH(CH₃) | OH | OH | H | ═O | *OCH₃ |
| 3,4-dihydroxy-7-methoxy- | | | | | | | | |
| (1,3)dioxolo(4,5-j)-3,4,4a,5- | | | | | | | | |
| tetrahydrophenanthridin-6(2H)- | | | | | | | | |
| one | | | | | | | | |
| (2R,3R,4S,4aR)-2-anilino-3,4- dihydroxy-7-methoxy- (1,3)dioxolo(4,5-j)-3,4,4a,5- tetrahydrophenanthridin-6(2H)- one | 173 | H | 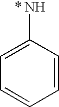 | OH | OH | H | ═O | *OCH₃ |
| (2R,3R,4S,4aR)-2- [methyl(phenyl)amino]-3,4- dihydroxy-7-methoxy- (1,3)dioxolo(4,5-j)-3,4,4a,5- tetrahydrophenanthridin-6(2H)- one | 174 | H | 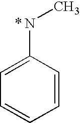 | OH | OH | H | ═O | *OCH₃ |
| methyl {[(2R,3R,4S,4aR)-2,3,4- | 175 | H | OH | OH | OH | H | ═O | *OCH₂CO₂Me |
| trihydroxy-(1,3)dioxolo(4,5-j)-6- | | | | | | | | |
| oxo-3,4,4a,5,- | | | | | | | | |
| tetrahydrophenanthridin-7- | | | | | | | | |
| yl]oxy}acetate | | | | | | | | |
| methyl {[(2R,3R,4S,4aR)-2,3,4- | 176 | H | *OAc | *OAc | *OAc | H | ═O | *OCH₂CO₂Me |
| tris[(methylcarbonyl)oxy]- | | | | | | | | |
| (1,3)dioxolo(4,5-j)-6-oxo- | | | | | | | | |
| 3,4,4a,5,- | | | | | | | | |
| tetrahydrophenanthridin-7- | | | | | | | | |
| yl]oxy}acetate | | | | | | | | |
| ethyl {[(2R,3R,4S,4aR)-2,3,4- | 177 | H | OH | OH | OH | H | ═O | *OCH₂CO₂Et |
| trihydroxy-(1,3)dioxolo(4,5-j)-6- | | | | | | | | |
| oxo-3,4,4a,5,- | | | | | | | | |
| tetrahydrophenanthridin-7- | | | | | | | | |
| yl]oxy}acetate | | | | | | | | |
| ethyl {[(2R,3R,4S,4aR)-2,3,4- | 178 | H | *OAc | *OAc | *OAc | H | ═O | *OCH₂CO₂Et |
| tris[(methylcarbonyl)oxy]- | | | | | | | | |
| (1,3)dioxolo(4,5-j)-6-oxo- | | | | | | | | |
| 3,4,4a,5,- | | | | | | | | |
| tetrahydrophenanthridin-7- | | | | | | | | |
| yl]oxy}acetate | | | | | | | | |
| {[(2R,3R,4S,4aR)-2,3,4- | 179 | H | OH | OH | OH | H | ═O | *OCH₂CO₂H |
| trihydroxy-(1,3)dioxolo(4,5-j)-6- | | | | | | | | |
| oxo-3,4,4a,5,- | | | | | | | | |
| tetrahydrophenanthridin-7- | | | | | | | | |
| yl]oxy}acetic acid | | | | | | | | |
| {[(2R,3R,4S,4aR)-2,3,4- | 180 | H | *OAc | *OAc | *OAc | H | ═O | *OCH₂CO₂H |
| tris[(methylcarbonyl)oxy]- | | | | | | | | |
| (1,3)dioxolo(4,5-j)-6-oxo- | | | | | | | | |
| 3,4,4a,5,- | | | | | | | | |
| tetrahydrophenanthridin-7- | | | | | | | | |
| yl]oxy}acetic acid | | | | | | | | |
| (2R,3R,4S,4aR)-7-(benzyloxy)- 2,3,4-trihydroxy- (1,3)dioxolo(4,5-j)-3,4,4a,5- tetrahydrophenanthridin-6(2H)- one | 181 | H | OH | OH | OH | H | ═O | 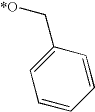 |
| (2R,3R,4S,4aR)-7-(benzyloxy)- 2,3,4-tris[(methylcarbonyl)oxy]- (1,3)dioxolo(4,5-j)-3,4,4a,5- tetrahydrophenanthridin-6(2H)- one | 182 | H | *OAc | *OAc | *OAc | H | ═O | 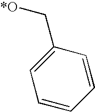 |
| (2R,3R,4S,4aR)-7-ethoxy-2,3,4- | 183 | H | OH | OH | OH | H | ═O | *OCH₂—CH₃ |
| trihydroxy-(1,3)dioxolo(4,5-j)- | | | | | | | | |
| 3,4,4a,5- | | | | | | | | |
| tetrahydrophenanthridin-6(2H)- | | | | | | | | |
| one | | | | | | | | |
| (2R,3R,4S,4aR)-7-ethoxy-2,3,4- | 184 | H | *OAc | *OAc | *OAc | H | ═O | *OCH₂—CH₃ |
| tris[(methylcarbonyl)oxy]- | | | | | | | | |
| (1,3)dioxolo(4,5-j)-3,4,4a,5- | | | | | | | | |
| tetrahydrophenanthridin-6(2H)- | | | | | | | | |
| one | | | | | | | | |
| (2R,3R,4S,4aR)-2,3,4- | 185 | H | OH | OH | OH | H | ═O | *OCH₂═CH₂ |
| trihydroxy-(1,3)dioxolo(4,5-j)-7- | | | | | | | | |
| (vinyloxy)-3,4,4a,5- | | | | | | | | |
| tetrahydrophenanthridin-6(2H)- | | | | | | | | |
| one | | | | | | | | |
| (2R,3R,4S,4aR)-2,3,4- | 186 | H | *OAc | *OAc | *OAc | H | ═O | *OCH₂═CH₂ |
| tris[(methylcarbonyl)oxy]- | | | | | | | | |
| (1,3)dioxolo(4,5-j)-7-(vinyloxy)- | | | | | | | | |
| 3,4,4a,5- | | | | | | | | |
| tetrahydrophenanthridin-6(2H)- | | | | | | | | |
| one | | | | | | | | |
| (2R,3R,4S,4aR)-7-[(3- fluorobenzyl)oxy]-2,3,4- trihydroxy-(1,3)dioxolo(4,5-j)- 3,4,4a,5- tetrahydrophenanthridin-6(2H)- one | 187 | H | OH | OH | OH | H | ═O | 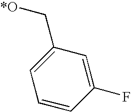 |
| (2R,3R,4S,4aR)-7-[(3- fluorobenzyl)oxy]-2,3,4- tris[(methylcarbonyl)oxy]- (1,3)dioxolo(4,5-j)-3,4,4a,5- tetrahydrophenanthridin-6(2H)- one | 188 | H | *OAc | *OAc | *OAc | H | ═O | 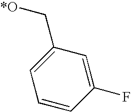 |
| (2R,3R,4S,4aR)-2,3,4- trihydroxy-7-[(4- nitrobenzyl)oxy]- (1,3)dioxolo(4,5-j)-3,4,4a,5- tetrahydrophenanthridin-6(2H)- one | 189 | H | OH | OH | OH | H | ═O | 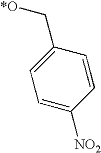 |
| (2R,3R,4S,4aR)-2,3,4- tris[(methylcarbonyl)oxy]-7-[(4- nitrobenzyl)oxy]- (1,3)dioxolo(4,5-j)-3,4,4a,5- tetrahydrophenanthridin-6(2H)- one | 190 | H | *OAc | *OAc | *OAc | H | ═O | 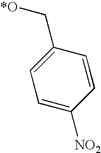 |
| (2R,3R,4S,4aR)-2,3,4- trihydroxy-7-[(2- naphthylmethoxy]- (1,3)dioxolo(4,5-j)-3,4,4a,5- tetrahydrophenanthridin-6(2H)- one - benzene (1:1) | 191 | H | OH | OH | OH | H | ═O | 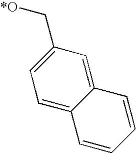 |
| (2R,3R,4S,4aR)-2,3,4- tris[(methylcarbonyl)oxy]-7-[(2- naphthylmethoxy]- (1,3)dioxolo(4,5-j)-3,4,4a,5- tetrahydrophenanthridin-6(2H)- one - benzene (1:1) | 192 | H | *OAc | *OAc | *OAc | H | ═O | 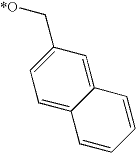 |
| (2R,3R,4S,4aR)-2,3,4- trihydroxy-7-[(4- methylbenzyl)oxy]- (1,3)dioxolo(4,5-j)-3,4,4a,5- tetrahydrophenanthridin-6(2H)- one | 193 | H | OH | OH | OH | H | ═O | 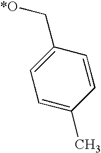 |
| (2R,3R,4S,4aR)-2,3,4- tris[(methylcarbonyl)oxy]-7-[(4- methylbenzyl)oxy]- (1,3)dioxolo(4,5-j)-3,4,4a,5- tetrahydrophenanthridin-6(2H)- one | 194 | H | *OAc | *OAc | *OAc | H | ═O | 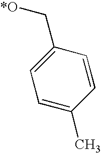 |
| (2R,3R,4S,4aR)-2,3,4- trihydroxy-7-[2-(2,5- dimethoxyphenyl)-2-oxoethoxy]- (1,3)dioxolo(4,5-j)-3,4,4a,5- tetrahydrophenanthridin-6(2H)- one | 195 | H | OH | OH | OH | H | ═O | 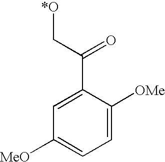 |
| (2R,3R,4S,4aR)-2,3,4- tris[(methylcarbonyl)oxy]-7-[2- (2,5-dimethoxyphenyl)-2- oxoethoxy]-(1,3)dioxolo(4,5-j)- 3,4,4a,5- tetrahydrophenanthridin-6(2H)- one | 196 | H | *OAc | *OAc | *OAc | H | ═O | 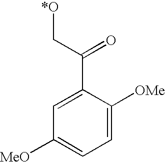 |
| (2R,3R,4S,4aR)-2,3,4- trihydroxy-7-[2-(3-nitrophenyl)- 2-oxoethoxy]-(1,3)dioxolo(4,5-j)- 3,4,4a,5- tetrahydrophenanthridin-6(2H)- one | 197 | H | OH | OH | OH | H | ═O | 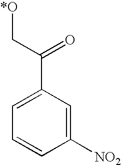 |
| (2R,3R,4S,4aR)-2,3,4- tris[(methylcarbonyl)oxy]-7-[2- (3-nitrophenyl)-2-oxoethoxy]- (1,3)dioxolo(4,5-j)-3,4,4a,5- tetrahydrophenanthridin-6(2H)- one | 198 | H | *OAc | *OAc | *OAc | H | ═O | 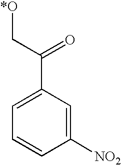 |
| sodium (2R,3R,4S,4aR)-2,3,4- | 199 | H | OH | OH | OH | H | ═O | *OSO₃Na |
| trihydroxy-(1,3)dioxolo(4,5-j)-6- | | | | | | | | |
| oxo-3,4,4a,5,- | | | | | | | | |
| tetrahydrophenanthridin-7-yl | | | | | | | | |
| sulfate | | | | | | | | |
| sodium (2R,3R,4S,4aR)-2,3,4- | 200 | H | *OAc | *OAc | *OAc | H | ═O | *OSO₃Na |
| tris[(methylcarbonyl)oxy]- | | | | | | | | |
| (1,3)dioxolo(4,5-j)-6-oxo- | | | | | | | | |
| 3,4,4a,5,- | | | | | | | | |
| tetrahydrophenanthridin-7-yl | | | | | | | | |
| sulfate | | | | | | | | |
| pyridinium (2R,3R,4S,4aR)- 2,3,4-trihydroxy- (1,3)dioxolo(4,5-j)-6-oxo- 3,4,4a,5,- tetrahydrophenanthridin-7-yl sulfate | 201 | H | OH | OH | OH | H | ═O | 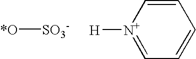 |
| pyridinium (2R,3R,4S,4aR)- 2,3,4-tris[(methylcarbonyl)oxy]- (1,3)dioxolo(4,5-j)-6-oxo- 3,4,4a,5,- tetrahydrophenanthridin-7-yl sulfate | 202 | H | *OAc | *OAc | *OAc | H | ═O | 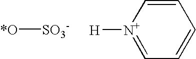 |
| (2R,3R,4S,4aR)-7- | 203 | H | *OAc | *OAc | *OAc | H | ═O | Tetrabenzoyl β-D- |
| {[(2S,3R,4S,5R,6R)-3,4,5- | | | | | | | | Glucosyl |
| tribenzoyloxy-6- | | | | | | | | |
| (benzoyloxymethyl)tetrahydro- | | | | | | | | |
| 2H-pyran-2-yl]oxy}- | | | | | | | | |
| (1,3)dioxolo(4,5-j)-6-oxo- | | | | | | | | |
| 2,3,4,4a,5,6- | | | | | | | | |
| hexahydrophenanthridin-2,3,4- | | | | | | | | |
| triyl triacetate | | | | | | | | |
| (2R,3R,4S,4aR)-2,3,4- | 204 | H | OH | OH | OH | H | ═O | β-D-Glucosyl |
| trihydroxy-7- | | | | | | | | |
| {[(2S,3R,4S,5S,6R)-3,4,5- | | | | | | | | |
| trihydroxy-6- | | | | | | | | |
| (hydroxymethyl)tetrahydro-2H- | | | | | | | | |
| pyran-2-yl]oxy}-(1,3)dioxolo(4,5- | | | | | | | | |
| j)-3,4,4a,5- | | | | | | | | |
| tetrahydrophenanthridine-6(2H)- | | | | | | | | |
| one | | | | | | | | |
| (2R,3R,4S,4aR)-7- | 205 | H | *OAc | *OAc | *OAc | H | ═O | Tribenzoyl β-L- |
| {[(2R,3R,4R,5S,6S)-3,4,5- | | | | | | | | Rhamnosyl |
| tribenzoyloxy-6- | | | | | | | | |
| methyltetrahydro-2H-pyran-2- | | | | | | | | |
| yl]oxy}-(1,3)dioxolo(4,5-j)-6-oxo- | | | | | | | | |
| 2,3,4,4a,5,6- | | | | | | | | |
| hexahydrophenanthridin-2,3,4- | | | | | | | | |
| triyl triacetate | | | | | | | | |
| (2R,3R,4S,4aR)-2,3,4- | 206 | H | OH | OH | OH | H | ═O | β-L-Rhamnosyl |
| trihydroxy-7- | | | | | | | | |
| {[(2R,3R,4R,5R,6S)-3,4,5- | | | | | | | | |
| trihydroxy-6-methyltetrahydro- | | | | | | | | |
| 2H-pyran-2-yl]oxy}- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3R,4S,4aR)-7- | 207 | H | *OAc | *OAc | *OAc | H | ═O | Tetrabenzoyl β-D- |
| {[(2R,3R,4S,5S,6R)-3,4,5- | | | | | | | | Mannosyl |
| tribenzyloxy-6- | | | | | | | | |
| (benzoyloxymethyl)tetrahydro- | | | | | | | | |
| 2H-pyran-2-yl]oxy}- | | | | | | | | |
| (1,3)dioxolo(4,5-j)-6-oxo- | | | | | | | | |
| 2,3,4,4a,5,6- | | | | | | | | |
| hexahydrophenanthridin-2,3,4- | | | | | | | | |
| triyl triacetate | | | | | | | | |
| (2R,3R,4S,4aR)-2,3,4- | 208 | H | OH | OH | OH | H | ═O | β-D-Mannosyl |
| trihydroxy-7- | | | | | | | | |
| {[(2S,3S,4S,5S,6R)-3,4,5- | | | | | | | | |
| trihydroxy-6- | | | | | | | | |
| (hydroxymethyl)tetrahydro-2H- | | | | | | | | |
| pyran-2-yl]oxy}-(1,3)dioxolo(4,5- | | | | | | | | |
| j)-3,4,4a,5- | | | | | | | | |
| tetrahydrophenanthridine-6(2H)- | | | | | | | | |
| one | | | | | | | | |
| (2R, 3R,4S,4aR)-7- | 209 | H | *OAc | *OAc | *OAc | H | ═O | Tribenzoyl β-L- |
| {[(2R,3S,4R,5R,6S)-3,4,5- | | | | | | | | Fucosyl |
| tribenzoyloxy-6- | | | | | | | | |
| methyltetrahydro-2H-pyran-2- | | | | | | | | |
| yl]oxy}-(1,3)dioxolo(4,5-j)-6-oxo- | | | | | | | | |
| 2,3,4,4a,5,6- | | | | | | | | |
| hexahydrophenanthridin-2,3,4- | | | | | | | | |
| triyl triacetate | | | | | | | | |
| (2R,3R,4S,4aR)-2,3,4- | 210 | H | OH | OH | OH | H | ═O | β-L-Fucosyl |
| trihydroxy-7- | | | | | | | | |
| {[(2R,3R,4R,5S,6S)-3,4,5- | | | | | | | | |
| trihydroxy-6-methyltetrahydro- | | | | | | | | |
| 2H-pyran-2-yl]oxy}- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3R,4S,4aR)-7- | 211 | H | *OAc | *OAc | *OAc | H | ═O | heptabenzoyl β-D- |
| {[(2S,3S,4S,5R,6R)-3,4,5- | | | | | | | | Isomaltosyl |
| tribenzoyloxy-6- | | | | | | | | |
| ({[(2S,3R,4S,5R,6R)-3,4,5- | | | | | | | | |
| tribenzoyloxy-6- | | | | | | | | |
| (benzoyloxymethyl)tetrahydro- | | | | | | | | |
| 2H-pyran-2- | | | | | | | | |
| yl]oxy}methyl)tetrahydro-2H- | | | | | | | | |
| pyran-2-yl]oxy}-(1,3)dioxolo(4,5- | | | | | | | | |
| j)-6-oxo-2,3,4,4a,5,6- | | | | | | | | |
| hexahydrophenanthridin-2,3,4- | | | | | | | | |
| triyl triacetate | | | | | | | | |
| (2R,3R,4S,4aR)-2,3,4- | 212 | H | OH | OH | OH | H | ═O | β-D-Isomaltosyl |
| trihydroxy-7- | | | | | | | | |
| {[(2R,3S,4S,5R,6S)-3,4,5- | | | | | | | | |
| trihydroxy-6- | | | | | | | | |
| ({[(2R,3S,4S,5R,6S)-3,4,5- | | | | | | | | |
| trihydroxy-6- | | | | | | | | |
| (hydroxymethyl)tetrahydro-2H- | | | | | | | | |
| pyran-2- | | | | | | | | |
| yl]oxy}methyl)tetrahydro-2H- | | | | | | | | |
| pyran-2-yl]oxy}-(1,3)dioxolo(4,5- | | | | | | | | |
| j)-3,4,4a,5- | | | | | | | | |
| tetrahydrophenanthridine-6(2H)- | | | | | | | | |
| one | | | | | | | | |
| (2R,3S,4S,4aR)-2,7- bis{[(2S,3R,4S,5R,6R))-3,4,5- tribenzoyloxy-6- 2H-pyran-2-yl]oxy}- (benzoyloxymethyl)tetrahydro- (1,3)dioxolo(4,5-j)-[2,2-dimethyl (1,3)dioxolo-(4,5-c)]-3,4,4a,5- tetrahydrophenanthridine-6(2H)- one | 213 | H | Tetrabenzoyl O-β-D-Glucosyl | 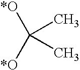 | | H | ═O | Tetrabenzoyl β-D-Glucosyl |
| (2R,3S,4S,4aR)-2,7- | 214 | H | Tetrabenzoyl | OH | OH | H | ═O | Tetrabenzoyl |
| bis{[(2S,3R,4S,5R,6R))-3,4,5- | | | O-β-D-Glucosyl | | | | | β-D-Glucosyl |
| tribenzoyloxy-6- | | | | | | | | |
| (benzoyloxymethyl)tetrahydro- | | | | | | | | |
| 2H-pyran-2-yl]oxy}-3,4- | | | | | | | | |
| dihydroxy-(1,3)dioxolo(4,5-j)- | | | | | | | | |
| 3,4,4a,5- | | | | | | | | |
| tetrahydrophenanthridine-6(2H)- | | | | | | | | |
| one | | | | | | | | |
| (2R,3S,4S,4aR)-2,7- | 215 | H | O-β-D-Glucosyl | OH | OH | H | ═O | β-D-Glucosyl |
| bis{[(2S,3R,4S,5S,6R))-3,4,5- | | | | | | | | |
| trihydroxy-6- | | | | | | | | |
| (hydroxymethyl)tetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3S,4S,4aR)-2,7- bis{[(2R,3R,4R,5S,6S)-3,4,5- tribenzoyloxy-6- methyltetrahydro-2H-pyran-2- yl]oxy}-(1,3)dioxolo(4,5-j)-[2,2- dimethyl(1,3)dioxolo-(4,5-c)]- 3,4,4a,5- tetrahydrophenanthridine-6(2H)- one | 216 | H | Tribenzoyl O-β-L- Rhamnosyl | 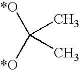 | | H | ═O | Tribenzoyl β-L- Rhamnosyl |
| (2R,3S,4S,4aR)-2,7- | 217 | H | Tribenzoyl O-β-L- | OH | OH | H | ═O | Tetrabenzoyl β-L- |
| bis{[(2R,3R,4R,5S,6S)-3,4,5- | | | Rhamnosyl | | | | | Rhamnosyl |
| tribenzoyloxy-6- | | | | | | | | |
| methyltetrahydro-2H-pyran-2- | | | | | | | | |
| yl]oxy}-3,4-dihydroxy- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3S,4S,4aR)-2,7- | 218 | H | O-β-L-Rhamnosyl | OH | OH | H | ═O | β-L-Rhamnosyl |
| bis{[(2R,3R,4R,5R,6S)-3,4,5- | | | | | | | | |
| trihydroxy-6-methyltetrahydro- | | | | | | | | |
| 2H-pyran-2-yl]oxy}-3,4- | | | | | | | | |
| dihydroxy-(1,3)dioxolo(4,5-j)- | | | | | | | | |
| 3,4,4a,5- | | | | | | | | |
| tetrahydrophenanthridine-6(2H)- | | | | | | | | |
| one | | | | | | | | |
| (2R,3S,4S,4aR)-2,7- bis{[(2R,3R,4S,5S,6R)-3,4,5- tribenzyloxy-6- (benzoyloxymethyl)tetrahydro- 2H-pyran-2-yl]oxy}- (1,3)dioxolo(4,5-j)-[2,2-dimethyl (1,3)dioxolo-(4,5-c)]-3,4,4a,5- tetrahydrophenanthridine-6(2H)- one | 219 | H | Tetrabenzoyl O-β-D- Mannosyl | 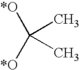 | | H | ═O | Tetrabenzoyl β-D- Mannosyl |
| (2R,3S,4S,4aR)-2,7- | 220 | H | Tetrabenzoyl O-β-D- | OH | OH | H | ═O | Tetrabenzoyl β-D- |
| bis{[(2R,3R,4S,5S,6R)-3,4,5- | | | Mannosyl | | | | | Mannosyl |
| tribenzyloxy-6- | | | | | | | | |
| (benzoyloxymethyl)tetrahydro- | | | | | | | | |
| 2H-pyran-2-yl]oxy}-3,4- | | | | | | | | |
| dihydroxy-(1,3)dioxolo(4,5-j)- | | | | | | | | |
| 3,4,4a,5- | | | | | | | | |
| tetrahydrophenanthridine-6(2H)- | | | | | | | | |
| one | | | | | | | | |
| (2R,3S,4S,4aR)-2,7- | 221 | H | O-β-D-Mannosyl | OH | OH | H | ═O | β-D-Mannosyl |
| bis{[(2S,3S,4S,5S,6R)-3,4,5- | | | | | | | | |
| trihydroxy-6- | | | | | | | | |
| (hydroxymethyl)tetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3S,4S,4aR)-2,7- bis{[(2R,3S,4R,5R,6S)-3,4,5- tribenzoyloxy-6- methyltetrahydro-2H-pyran-2- yl]oxy}-(1,3)dioxolo(4,5-j)-[2,2- dimethyl(1,3)dioxolo-(4,5-c)]- 3,4,4a,5- tetrahydrophenanthridine-6(2H)- one | 222 | H | Tribenzoyl O-β-L- Fucosyl | 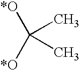 | | H | ═O | Tribenzoyl β-L- Fucosyl |
| (2R,3S,4S,4aR)-2,7- | 223 | H | Tribenzoyl O-β-L- | OH | OH | H | ═O | Tribenzoyl β-L- |
| bis{[(2R,3S,4R,5R,6S)-3,4,5- | | | Fucosyl | | | | | Fucosyl |
| tribenzoyloxy-6- | | | | | | | | |
| methyltetrahydro-2H-pyran-2- | | | | | | | | |
| yl]oxy}-3,4-dihydroxy- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3S,4S,4aR)-2,7- | 224 | H | O-β-L-Fucosyl | OH | OH | H | ═O | β-L-Fucosyl |
| bis{[(2R,3R,4R,5S,6S)-3,4,5- | | | | | | | | |
| trihydroxy-6-methyltetrahydro- | | | | | | | | |
| 2H-pyran-2-yl]oxy}-3,4- | | | | | | | | |
| dihydroxy-(1,3)dioxolo(4,5-j)- | | | | | | | | |
| 3,4,4a,5- | | | | | | | | |
| tetrahydrophenanthridine-6(2H)- | | | | | | | | |
| one | | | | | | | | |
| (2R,3S,4S,4aR)-2,7- bis{[(2S,3S,4S,5R,6R)-3,4,5- tribenzoyloxy-6- ({[(2S,3R,4S,5R,6R)-3,4,5- tribenzoyloxy-6- (benzoyloxymethyl)tetrahydro- 2H-pyran-2- yl]oxy}methyl)tetrahydro-2H- pyran-2-yl]oxy}-(1,3)dioxolo(4,5- j)-[2,2-dimethyl(1,3)dioxolo- (4,5-c)]-3,4,4a,5- tetrahydrophenanthridine-6(2H)- one | 225 | H | heptabenzoyl O-β-D- Isomaltosyl | 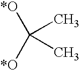 | | H | ═O | heptabenzoyl β-D- Isomaltosyl |
| (2R,3S,4S,4aR)-2,7- | 226 | H | heptabenzoyl O-β-D- | OH | OH | H | ═O | heptabenzoyl β-D- |
| bis{[(2S,3S,4S,5R,6R)-3,4,5- | | | Isomaltosyl | | | | | Isomaltosyl |
| tribenzoyloxy-6- | | | | | | | | |
| ({[(2S,3R,4S,5R,6R)-3,4,5- | | | | | | | | |
| tribenzoyloxy-6- | | | | | | | | |
| (benzoyloxymethyl)tetrahydro- | | | | | | | | |
| 2H-pyran-2- | | | | | | | | |
| yl]oxy}methyl)tetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3S,4S,4aR)-2,7- | 227 | H | O-β-D-Isomaltosyl | OH | OH | H | ═O | β-D-Isomaltosyl |
| bis{[(2R,3S,4S,5R,6S)-3,4,5- | | | | | | | | |
| trihydroxy-6- | | | | | | | | |
| ({[(2R,3S,4S,5R,6S)-3,4,5- | | | | | | | | |
| trihydroxy-6- | | | | | | | | |
| (hydroxymethyl)tetrahydro-2H- | | | | | | | | |
| pyran-2- | | | | | | | | |
| yl]oxy}methyl)tetrahydro-2H- | | | | | | | | |
| pyran-2-yl]oxy}-3,4-dihydroxy- | | | | | | | | |
| (1,3)dioxolo(4,5-j)-3,4,4a,5- | | | | | | | | |
| tetrahydrophenanthridine-6(2H)- | | | | | | | | |
| one | | | | | | | | |
| (2R,3S,4S,4aR)-2,7-bis{N- methylaminocarbonyloxy}-3,4- dihydroxy-(1,3)dioxolo(4,5-j)- 3,4,4a,5- tetrahydrophenanthridine-6(2H)- one | 228 | H | 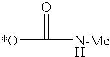 | OH | OH | H | ═O | 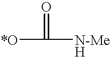 |
| (2R,3S,4S,4aR)-2-{N- methylaminocarbonyloxy}-3,4,7- trihydroxy-(1,3)dioxolo(4,5-j)- 3,4,4a,5- tetrahydrophenanthridine-6(2H)- one | 229 | H | 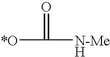 | OH | OH | H | ═O | OH |
| (2R,3S,4S,4aR)-2,7-bis{N- methylaminocarbonyloxy}- (1,3)dioxolo(4,5-j)-[2,2-dimethyl (1,3)dioxolo-(4,5-c)]-3,4,4a,5- tetrahydrophenanthridine-6(2H)- one | 230 | H | 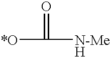 | 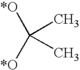 | | H | ═O | 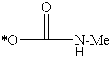 |
| (2R,3S,4S,4aR)-2,7-bis(N- ethylaminocarbonyloxy)-3,4- dihydroxy-(1,3)dioxolo(4,5-j)- 3,4,4a,5- tetrahydrophenanthridine-6(2H)- one | 231 | H | 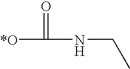 | OH | OH | H | ═O | 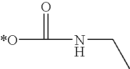 |
| (2R,3S,4S,4aR)-2-{N- ethylaminocarbonyloxy}-3,4,7- trihydroxy-(1,3)dioxolo(4,5-j)- 3,4,4a,5- tetrahydrophenanthridine-6(2H)- one | 232 | H | 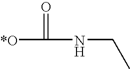 | OH | OH | H | ═O | OH |
| (2R,3S,4S,4aR)-2,7-bis{N- ethylaminocarbonyloxy}- (1,3)dioxolo(4,5-j)-[2,2-dimethyl (1,3)dioxolo-(4,5-c)]-3,4,4a,5- tetrahydrophenanthridine-6(2H)- one | 233 | H | 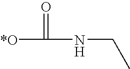 | 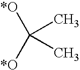 | | H | ═O | 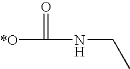 |
| (2R,3S,4S,4aR)-2,7-bis(N- isopropylaminocarbonyloxy)- 3,4-dihydroxy-(1,3)dioxolo(4,5- j)-3,4,4a,5- tetrahydrophenanthridine-6(2H)- one | 234 | H | 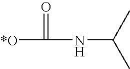 | OH | OH | H | ═O | 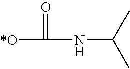 |
| (2R,3S,4S,4aR)-2,7-bis{N- isopropylaminocarbonyloxy}- (1,3)dioxolo(4,5-j)-[2,2-dimethyl (1,3)dioxolo-(4,5-c)]-3,4,4a,5- tetrahydrophenanthridine-6(2H)- one | 235 | H | 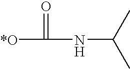 | 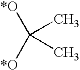 | | H | ═O | 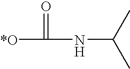 |
| (2R,3S,4S,4aR)-2-{N- isopropylaminocarbonyloxy}- 3,4,7-trihydroxy- (1,3)dioxolo(4,5-j)-3,4,4a,5- tetrahydrophenanthridine-6(2H)- one | 236 | H | 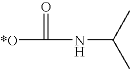 | OH | OH | H | ═O | OH |
| (2R,3S,4S,4aR)-2,7-bis(N- phenylaminocarbonyloxy)-3,4- dihydroxy-(1,3)dioxolo(4,5-j)- 3,4,4a,5- tetrahydrophenanthridine-6(2H)- one | 237 | H | 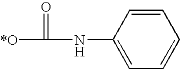 | OH | OH | H | ═O | 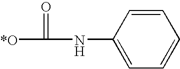 |
| (2R,3S,4S,4aR)-2,7-bis{N- phenylaminocarbonyloxy}- (1,3)dioxolo(4,5-j)-[2,2-dimethyl (1,3)dioxolo-(4,5-c)]-3,4,4a,5- tetrahydrophenanthridine-6(2H)- one | 238 | H | 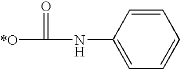 | 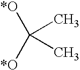 | | H | ═O | 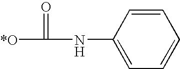 |
| (2R,3S,4S,4aR)-2-{N- phenylaminocarbonyloxy}-3,4,7- trihydroxy-(1,3)dioxolo(4,5-j)- 3,4,4a,5- tetrahydrophenanthridine-6(2H)- one | 239 | H | 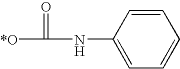 | OH | OH | H | ═O | OH |
| (2R,3S,4S,4aR)-2,7- bis(ethylcarbonyloxy)-3,4- dihydroxy-(1,3)dioxolo(4,5-j)- 3,4,4a,5- tetrahydrophenanthridine-6(2H)- one | 240 | H | 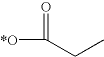 | OH | OH | H | ═O | 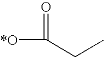 |
| (2R,3S,4S,4aR)-2,7- bis{ethylcarbonyloxy}- (1,3)dioxoio(4,5-j)-[2,2-dimethyl (1,3)dioxolo-(4,5-c)]-3,4,4a,5- tetrahydrophenanthridine-6(2H)- one | 241 | H | 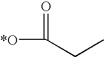 | 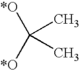 | | H | ═O | 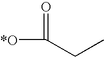 |
| (2R,3S,4S,4aR)-2-{N- ethylcarbonyloxy}-3,4,7- trihydroxy-(1,3)dioxolo(4,5-j)- 3,4,4a,5- tetrahydrophenanthridine-6(2H)- one | 242 | H | 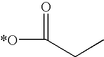 | OH | OH | H | ═O | OH |
| (2R,3S,4S,4aR)-2,7- bis(isopropylcarbonyloxy)-3,4- dihydroxy-(1,3)dioxolo(4,5-j)- 3,4,4a,5- tetrahydrophenanthridine-6(2H)- one | 243 | H | 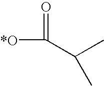 | OH | OH | H | ═O | 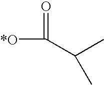 |
| (2R,3S,4S,4aR)-2,7- bis{isopropylcarbonyloxy}- (1,3)dioxolo(4,5-j)-[2,2-dimethyl (1,3)dioxolo-(4,5-c)]-3,4,4a,5- tetrahydrophenanthridine-6(2H)- one | 244 | H | 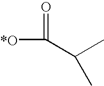 | 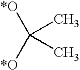 | | H | ═O | 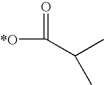 |
| (2R,3S,4S,4aR)-2- {isopropylcarbonyloxy}-3,4,7- trihydroxy-(1,3)dioxolo(4,5-j)- 3,4,4a,5- tetrahydrophenanthridine-6(2H)- one | 245 | H | 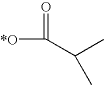 | OH | OH | H | ═O | OH |
| (2R,3S,4S,4aR)-2,7-bis(prop-1- en-carbonyloxy)-3,4-dihydroxy- (1,3)dioxolo(4,5-j)-3,4,4a,5- tetrahydrophenanthridine-6(2H)- one | 246 | H | 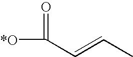 | OH | OH | H | ═O | 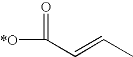 |
| (2R,3S,4S,4aR)-2,7-bis{prop-1- en-carbonyloxy}- (1,3)dioxolo(4,5-j)-[2,2-dimethyl (1,3)dioxolo-(4,5-c)]-3,4,4a,5- tetrahydrophenanthridine-6(2H)- one | 247 | H | 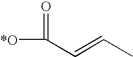 | 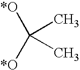 | | H | ═O | 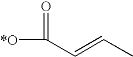 |
| (2R,3S,4S,4aR)-2-{prop-1-en- carbonyloxy}-3,4,7-trihydroxy- (1,3)dioxolo(4,5-j)-3,4,4a,5- tetrahydrophenanthridne-6(2H)- one | 248 | H | 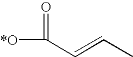 | OH | OH | H | ═O | OH |
| (2R,3S,4S,4aR)-2,7-bis(2- methylpropan-2-carbonyloxy)- 3,4-dihydroxy-(1,3)dioxolo(4,5- j)-3,4,4a,5- tetrahydrophenanthridine-6(2H)- one | 249 | H | 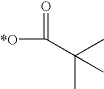 | OH | OH | H | ═O | 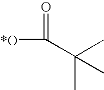 |
| (2R,3S,4S,4aR)-2,7-bis{2- methylpropan-2-carbonyloxy}- (1,3)dioxolo(4,5-j)-[2,2-dimethyl (1,3)dioxolo-(4,5-c)]-3,4,4a,5- tetrahydrophenanthridine-6(2H)- one | 250 | H | 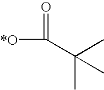 | 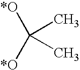 | | | | 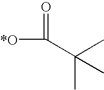 |
| (2R,3S,4S,4aR)-2-{2- methylpropan-2-carbonyloxy}- 3,4,7-trihydroxy- (1,3)dioxolo(3,4,4a,5- tetrahydrophenanthridine-6(2H)- one | 251 | H | 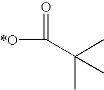 | OH | OH | H | ═O | OH |
| (2R,3S,4S,4aR)-2,7-bis(N- methylaminothocarbonyloxy)- 3,4-dihydroxy-(1,3)dioxolo(4,5- j)-3,4,4a,5- tetrahydrophenanthridine-6(2H)- one | 252 | H | 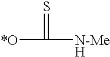 | OH | OH | H | ═O | 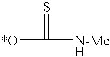 |
| (2R,3S,4S,4aR)-2-{N- methylaminothiocarbonyloxy}- 3,4,7-trihydroxy- (1,3)dioxolo(4,5-j)-3,4,4a,5- tetrahydrophenanthridine-6(2H)- one | 253 | H | 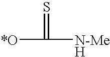 | OH | OH | H | ═O | OH |
| (2R,3S,4S,4aR)-2,7-bis{N- methylaminothiocarbonyloxy}- (1,3)dioxolo(4,5-j)-[2,2-dimethyl (1,3)dioxolo-(4,5-c)]-3,4,4a,5- tetrahydrophenanthridine-6(2H)- one | 254 | H | 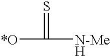 | 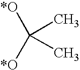 | | H | ═O | 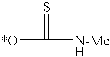 |
| (2R,3S,4S,4aR)-2,7-bis(N- ethylaminothiocarbonyloxy)-3,4- dihydroxy-(1,3)dioxolo(4,5-j)- 3,4,4a,5- tetrahydrophenanthridine-6(2H)- one | 255 | H | 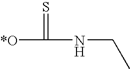 | OH | OH | H | ═O | 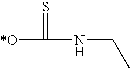 |
| (2R,3S,4S,4aR)-2-{N- ethylaminothiocarbonyloxy}- 3,4,7-trihydroxy- (1,3)dioxolo(4,5-j)-3,4,4a,5- tetrahydrophenanthridine-6(2H)- one | 256 | H | 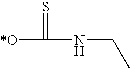 | OH | OH | H | ═O | OH |

| | | | | | | | |
|---|---|---|---|---|---|---|---|
| (2R,3S,4S,4aR)-2,7-bis{N- ethylaminothiocarbonyloxy}- (1,3)dioxolo(4,5-j)-[2,2-dimethyl (1,3)dioxolo-(4,5-c)]-3,4,4a,5- tetrahydrophenanthridine-6(2H)- one | 257 | H | 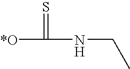 | 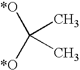 | H | ═O | 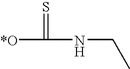 |

TABLE 3

| | |
|---|---|
| | VI |
| 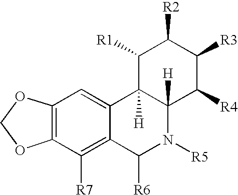 | |

| name | Compound | R¹ | R² | R³ | R⁴ | R⁵ | R⁶ | R⁷ |
|---|---|---|---|---|---|---|---|---|
| (2R,3R,4S,4aR,10bR)-7- hydroxy-[2,2-dimethyl (1,3)dioxolo-(4,5-c)- (1,3)dioxolo(4,5-j)-2-amino- 1,3,4,4a,5,10b- hexahydrophenanthridin-6(2H)- one | 258 | H | NH₂ | 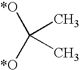 | | H | ═O | OH |
| (2R,3R,4S,4aR,10bR)-3,4,7- | 259 | H | NH₂ | OH | OH | H | ═O | OH |
| trihydroxy-(1,3)dioxolo(4,5-j)-2- | | | | | | | | |
| (amino)-1,3,4,4a,5,10b- | | | | | | | | |
| hexahydrophenanthridin-6(2H)- | | | | | | | | |
| one | | | | | | | | |
| (2R,3R,4S,4aR,10bR)-7- hydroxy-[2,2-dimethyl (1,3)dioxolo-(4,5-c)- (1,3)dioxolo(4,5-j)-2- (propylamino)-1,3,4,4a,5,10b- hexahydrophenanthridin-6(2H)- one | 260 | H | 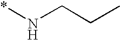 | 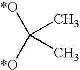 | | H | ═O | OH |
| (2R,3R,4S,4aR,10bR)-3,4,7- trihydroxy-(1,3)d ioxolo(4,5-j)-2- (propylamino)-1,3,4,4a,5,10b- hexahydrophenanthridin-6(2H)- one | 261 | H | 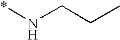 | OH | OH | H | ═O | OH |
| (1S,2S,3S,4S,4aR,10bR)- | 262 | *OAc— | OH | OH | OH | H | ═O | OH |
| 2,3,4,7-tetrahydroxy- | | | | | | | | |
| (1,3)dioxolo(4,5-j)-6-oxo- | | | | | | | | |
| 1,2,3,4,4a,5,6,10b- | | | | | | | | |
| octahydrophenanthridin-1-yl | | | | | | | | |
| acetate | | | | | | | | |
| (1R,2R,3R,4S,4aR,10bR)- (1,3)dioxolo(4,5-j)-7-hydroxy-1- phenyl-6-oxo- 1,2,3,4,4a,5,6,10b- octahydrophenanthridine-2,3,4- triyl triacetate | 263 | 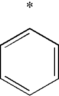 | *OAc | *OAc | *OAc | H | ═O | OH |
| (1R,2R,3R,4S,4aR,10bR)- 2,3,4,7-tetrahydroxy- (1,3)dioxolo(4,5-j)-1-phenyl- 1,3,4,4a,5,10b- hexahydrophenanthridin-6(2H)- one | 264 | 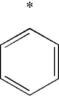 | OH | OH | OH | H | ═O | OH |

TABLE 4

| | |
|---|---|
| | V |
| 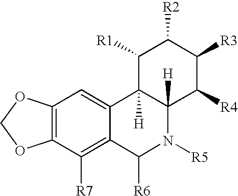 | |

| name | Compound | R¹ | R² | R³ | R⁴ | R⁵ | R⁶ | R⁷ |
|---|---|---|---|---|---|---|---|---|
| (2S,3R,4S,4aR,10bR)-2,3,4,7- | 265 | H | OH | OH | OH | H | ═O | OMe |
| tetrahydroxy-(1,3)dioxolo(4,5-j)- | | | | | | | | |
| 1,3,4,4a,5,10b- | | | | | | | | |
| hexahydrophenanthridin-6(2H)-one | | | | | | | | |
| (2S,3R,4S,4aR,10bR)-7-hydroxy- [2,2-dimethyl(1,3)dioxolo-(4,5-c)- (1,3)dioxolo(4,5-j)-2-oxo- 1,3,4,4a,5,10b- hexahydrophenanthridin-6(2H)-one | 266 | H | ═O | 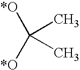 | | H | ═O | OH |
| (2S,3R,4S,4aR,10bR)-7-hydroxy- [2,2-dimethyl(1,3)dioxolo-(4,5-c)- (1,3)dioxolo(4,5-j)-2-amino- 1,3,4,4a,5,10b- hexahydrophenanthridin-6(2H)-one | 267 | H | NH₂ | 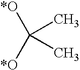 | | H | ═O | OH |
| (2S,3R,4S,4aR,10bR)-3,4,7- | 268 | H | ═O | OH | OH | H | ═O | OH |
| trihydroxy-(1,3)dioxolo(4,5-j)-2-oxo- | | | | | | | | |
| 1,3,4,4a,5,10b- | | | | | | | | |
| hexahydrophenanthridin-6(2H)-one | | | | | | | | |
| (2S,3R,4S,4aR,10bR)-3,4,7- | 269 | H | NH₂ | OH | OH | H | ═O | OH |
| trihydroxy-(1,3)dioxolo(4,5-j)-2- | | | | | | | | |
| (amino)-1,3,4,4a,5,10b- | | | | | | | | |
| hexahydrophenanthridin-6(2H)-one | | | | | | | | |
| (2S,3R,4S,4aR,10bR)-7-hydroxy- [2,2-dimethyl(1,3)dioxolo-(4,5-c)- (1,3)dioxolo(4,5-j)-2-(propylamino)- 1,3,4,4a,5,10b- hexahydrophenanthridin-6(2H)-one | 270 | H | 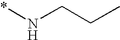 | 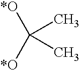 | | H | ═O | OH |
| (2S,3R,4S,4aR,10bR)-3,4,7- trihydroxy-(1,3)dioxolo(4,5-j)-2- (propylamino)-1,3,4,4a,5,10b- hexahydrophenanthridin-6(2H)-one | 271 | H | 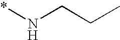 | OH | OH | H | ═O | OH |
| (1S,2R,3S,4S,4aR,10bR)-2,3,4,7- | 272 | *OAc— | OH | OH | OH | H | ═O | OH |
| tetrahydroxy-(1,3)dioxolo(4,5-j)-6- | | | | | | | | |
| oxo-1,2,3,4,4a,5,6,10b- | | | | | | | | |
| octahydrophenanthridin-1-yl | | | | | | | | |
| acetate | | | | | | | | |
| (1R,2S,3R,4S,4aR,10bR)- (1,3)dioxolo(4,5-j)-7-hydroxy-1- phenyl-6-oxo-1,2,3,4,4a,5,6,10b- octahydrophenanthridine-2,3,4-triyl triacetate | 273 | 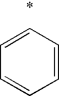 | *OAc | *OAc | *OAc | H | ═O | OH |
| (1R,2S,3R,4S,4aR,10bR)-2,3,4,7- tetrahydroxy-(1,3)dioxolo(4,5-j)-1- phenyl-1,3,4,4a,5,10b- hexahydrophenanthridin-6(2H)-one | 274 | 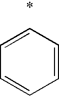 | OH | OH | OH | H | ═O | OH |

TABLE 5

| | | | | | | | | VIII |
|---|---|---|---|---|---|---|---|---|
| 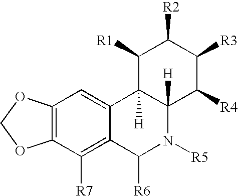 | | | | | | | | |
| name | Compound | R¹ | R² | R³ | R⁴ | R⁵ | R⁶ | R⁷ |
| (1R,2S,3S,4S,4aR,10bR)-2,3,4,7- | 275 | *OAc— | OH | OH | OH | H | ═O | OH |
| tetrahydroxy-(1,3)dioxolo(4,5-j)-6- | | | | | | | | |
| oxo-1,2,3,4,4a,5,6,10b- | | | | | | | | |
| octahydrophenanthridin-1-yl acetate | | | | | | | | |
| (1S,2R,3R,4S,4aR,10bR)- (1,3)dioxolo(4,5-j)-7-hydroxy-1- phenyl-6-oxo-1,2,3,4,4a,5,6,10b- octahydrophenanthridine-2,3,4-triyl triacetate | 276 | 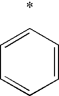 | *OAc | *OAc | *OAc | H | ═O | OH |
| (1S,2R,3R,4S,4aR,10bR)-2,3,4,7- tetrahydroxy-(1,3)dioxolo(4,5-j)-1- phenyl-1,3,4,4a,5,10b- hexahydrophenanthridin-6(2H)-one | 277 | 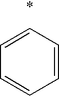 | OH | OH | OH | H | ═O | OH |

TABLE 6

| | |
|---|---|
| | VII |
| 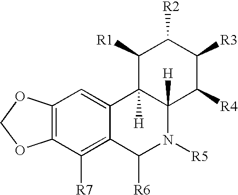 | |

| name | Compound | R¹ | R² | R³ | R⁴ | R⁵ | R⁶ | R⁷ |
|---|---|---|---|---|---|---|---|---|
| (1R,2R,3S,4S,4aR,10bR)-2,3,4,7- | 278 | *OAc— | OH | OH | OH | H | ═O | OH |
| tetrahydroxy-(1,3)dioxolo(4,5-j)-6-oxo- | | | | | | | | |
| 1,2,3,4,4a,5,6,10b- | | | | | | | | |
| octahydrophenanthridin-1-yl acetate | | | | | | | | |
| (1S,2R,3R,4S,4aR,10bR)- (1,3)dioxolo(4,5-j)-7-hydroxy-1-phenyl-6- oxo-1,2,3,4,4a,5,6,10b- octahydrophenanthridine-2,3,4-triyl triacetate | 279 | 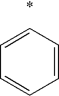 | *OAc | *OAc | *OAc | H | ═O | OH |
| (1S,2S,3R,4S,4aR,10bR)-2,3,4,7- tetrahydroxy-(1,3)dioxolo(4,5-j)-1-phenyl- 1,3,4,4a,5,10b-hexahydrophenanthridin- 6(2H)-one | 280 | 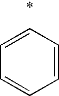 | OH | OH | OH | H | ═O | OH |
| (2S,3R,4S,4aR,10bR)-7-hydroxy- | 281 | H | *OAc | *OAc | *OAc | H | ═S | OH |
| (1,3)dioxolo(4,5-j-6-thioxo- | | | | | | | | |
| 1,2,3,4,4a,5,6,10b- | | | | | | | | |
| octahydrophenanthridine-2,3,4-triyl | | | | | | | | |
| triacetate | | | | | | | | |
| methyl (2S,3R,4S,4aR,10bR)-2,3,4- | 282 | H | OH | OH | OH | H | ═O | *OCH₂CO₂Me |
| trihydroxy-(1,3)dioxolo(4,5-j)-6-oxo- | | | | | | | | |
| 1,2,3,4,4a,5,6,10b- | | | | | | | | |
| octahydrophenanthridin-7-yl carbonate | | | | | | | | |
| methyl {[(2S,3R,4S,4aR,10bR)-2,3,4- | 283 | H | *OAc | *OAc | *OAc | H | ═O | *OCH₂CO₂Me |
| tris[(methylcarbonyl)oxy]- | | | | | | | | |
| (1,3)dioxolo(4,5-j)-6-oxo- | | | | | | | | |
| 1,2,3,4,4a,5,6,10b- | | | | | | | | |
| octahydrophenanthridin-7-yl]oxy}acetate | | | | | | | | |
| ethyl {(2S,3R,4S,4aR,10bR)-2,3,4- | 284 | H | OH | OH | OH | H | ═O | *OCH₂CO₂Et |
| trihydroxy-(1,3)dioxolo(4,5-j)-6-oxo- | | | | | | | | |
| 1,2,3,4,4a,5,6,10b- | | | | | | | | |
| octahydrophenanthridin-7-yl]oxy}acetate | | | | | | | | |
| ethyl {[(2S,3R,4S,4aR,10bR)-2,3,4- | 285 | H | *OAc | *OAc | *OAc | H | ═O | *OCH₂CO₂Et |
| tris[(methylcarbonyl)oxy]- | | | | | | | | |
| (1,3)dioxolo(4,5-j)-6-oxo- | | | | | | | | |
| 1,2,3,4,4a,5,6,10b- | | | | | | | | |
| octahydrophenanthridin-7-yl]oxy}acetate | | | | | | | | |
| {[(2S,3R,4S,4aR,10bR)-2,3,4-trihydroxy- | 286 | H | OH | OH | OH | H | ═O | *OCH₂CO₂H |
| (1,3)dioxolo(4,5-j)-6-oxo- | | | | | | | | |
| 1,2,3,4,4a,5,6,10b- | | | | | | | | |
| octahydrophenanthridin-7-yl]oxy}acetic | | | | | | | | |
| acid | | | | | | | | |
| {[(2S,3R,4S,4aR,10bR)-2,3,4- | 287 | H | *OAc | *OAc | *OAc | H | ═O | *OCH₂CO₂H |
| tris[(methylcarbonyl)oxy]- | | | | | | | | |
| (1,3)dioxolo(4,5-j)-6-oxo- | | | | | | | | |
| 1,2,3,4,4a,5,6,10b- | | | | | | | | |
| octahydrophenanthridin-7-yl]oxy}acetic | | | | | | | | |
| acid | | | | | | | | |
| (2S,3R,4S,4aR,10bR)-7-(benzyloxy)- 2,3,4-trihydroxy-(1,3)dioxolo(4,5-j)- 1,3,4,4a,5,10b-hexahydrophenanthridin- 6(2H)-one | 288 | H | OH | OH | OH | H | ═O | 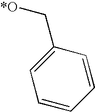 |
| (2S,3R,4S,4aR,10bR)-7-(benzyloxy)- (1,3)dioxolo(4,5-j)-6-oxo- 1,2,3,4,4a,5,6,10b- octahydrophenanthridine-2,3,4-triyl triacetate | 289 | H | *OAc | *OAc | *OAc | H | ═O | 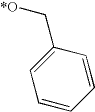 |
| (2S,3R,4S,4aR,10bR)-7-ethoxy-2,3,4- | 290 | H | OH | OH | OH | H | ═O | *OCH₂—CH₃ |
| trihydroxy-(1,3)dioxolo(4,5-j)- | | | | | | | | |
| 1,3,4,4a,5,10b-hexahydrophenanthridin- | | | | | | | | |
| 6(2H)-one | | | | | | | | |
| (2S,3R,4S,4aR,10bR)-7-ethoxy- | 291 | H | *OAc | *OAc | *OAc | H | ═O | *OCH₂—CH₃ |
| (1,3)dioxolo(4,5-j)-6-oxo- | | | | | | | | |
| 1,2,3,4,4a,5,6,10b- | | | | | | | | |
| octahydrophenanthridine-2,3,4-triyl | | | | | | | | |
| triacetate | | | | | | | | |
| (2S,3R,4S,4aR,10bR)-2,3,4-trihydroxy- | 292 | H | OH | OH | OH | H | ═O | *OCH₂═CH₂ |
| (1,3)dioxolo(4,5-j)-7-(vinyloxy)- | | | | | | | | |
| 1,3,4,4a,5,10b-hexahydrophenanthridin- | | | | | | | | |
| 6(2H)-one | | | | | | | | |
| (2R,3S,4S,4aR,10bR)-(1,3)dioxolo(4,5- | 293 | H | *OAc | *OAc | *OAc | H | ═O | *OCH₂═CH₂ |
| j)-7-(vinyloxy)-6-oxo-1,2,3,4,4a,5,6,10b- | | | | | | | | |
| octahydrophenanthridine-2,3,4-triyl | | | | | | | | |
| triacetate | | | | | | | | |
| (2R,3S,4S,4aR,10bR)-7-[(3- fluorobenzyl)oxy]-2,3,4-trihydroxy- (1,3)dioxolo(4,5-j)-1,3,4,4a,5,10b- hexahydrophenanthridin-6(2H)-one | 294 | H | OH | OH | OH | H | ═O | 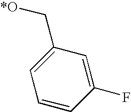 |
| (2R,3S,4S,4aR,10bR)-7-[(3- fluorobenzyl)oxy]-(1,3)dioxolo(4,5-j)-6- oxo-1,2,3,4,4a,5,6,10b- octahydrophenanthridine-2,3,4-triyl triacetate | 295 | H | *OAc | *OAc | *OAc | H | ═O | 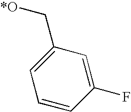 |
| (2R,3S,4S,4aR,10bR)-2,3,4-trihydroxy-7- [(4-nitrobenzyl)oxy]-(1,3)dioxolo(4,5-j)- 1,3,4,4a,5,10b-hexahydrophenanthridin- 6(2H)-one | 296 | H | OH | OH | OH | H | ═O | 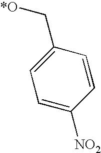 |

The present invention encompasses compounds 1 to 296 and stereoisomers, tautomers, racemates or a pharmaceutically acceptable salt and/or solvate thereof.

### Example 2

### Preparation of the Compounds According to the Invention

The following procedures were used to prepare the compounds described in the present invention.

### Compound 1:

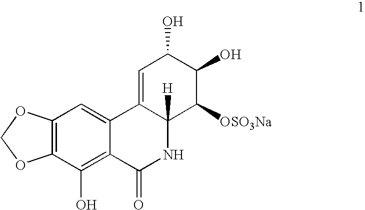

Narciclasine (50 mg, 163 μmol) and Bu₂SnO (41 mg, 163 μmol) were stirred in refluxing MeOH (3 ml of extra dry) under nitrogen for 3 h. The solvent was removed in vacuo and the dry dibutylstannylene complex was dissolved in extra dry DMF (1 ml). Me₃N.SO₃ (45 mg, 326 μmol) was added and the reaction mixture was stirred at room temperature for 28 h. MeOH (1 ml) and a cation exchange resin (Dowex, Na⁺, 1×4 cm) were added and the mixture was stirred for 30 min, filtered and reduced in vacuo. The residue was freeze-dried for 48 h. Chromatography (CH₂Cl₂/MeOH/H₂O 90:10:1 to 70:30:5) gave compound 1 (62 mg, 93%). Rf 0.43 (CH₂Cl₂/MeOH/H₂O 70:30:5).

¹H NMR (DMSO-d6): δ 3.83 (m, 1H), 4.02 (m, 1H), 4.34 (m, 1H), 4.55 (dd, J 8.7, 2.1, 1H), 5.30 (m, 2H), 6.09 (s, 2H), 6.16 (m, 1H), 6.85 (s, 1H), 8.33 (s, 1H), 13.14 (s, 1H). ¹³C NMR (DMSO-d6): δ 51.51, 69.18, 71.10, 74.36, 96.03, 102.03, 105.59, 124.29, 129.39, 131.89, 133.45, 144.66, 152.21, 168.19.

MS (ESI) m/z 386 (M⁻−Na).

### Compound 2 and Compound 3:

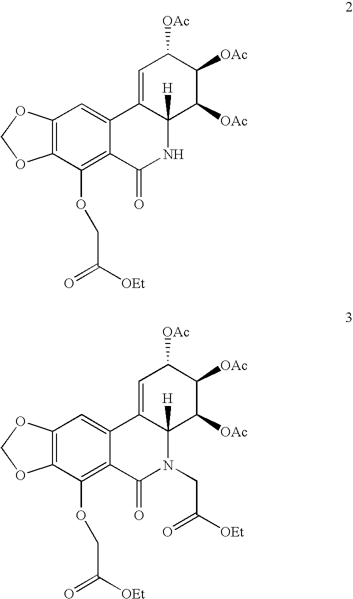

2,3,4-(triacetoxy)-narciclasine (30 mg, 69 μmol) and ethyl bromoacetate (23 mg, 138 μmol) was stirred at room temperature in extra dry DMF then K₂CO₃ (10 mg, 72 μmol) was added. The reaction mixture was then heated to 60° C. for 22 h and then this mixture was evaporated and freeze-dried for 24 h. Chromatography (CH₂Cl₂/MeOH 99:1 to 96:4) gave compound 2 (22 mg, 61%) Rf 0.14 (CH₂Cl₂/MeOH 96:4) and compound 3 (13 mg, 30%) Rf 0.29 (CH₂Cl₂/MeOH 96:4)

compound 2: ¹H NMR (DMSO-d6): δ 1.20 (t, 3H), 1.99 (s, 3H), 2.04 (s, 3H), 2.09 (s, 3H), 4.15 (q, 2H), 4.42 (m, 1H), 4.74 (s, 2H), 4.98 (m, 1H), 5.29 (m, 2H), 6.08 (1s, 1H), 6.13 (s, 1H), 6.27 (m, 1H), 7.11 (s, 1H), 8.31 (s, 1H). ¹³C NMR (DMSO-d6): δ 13.95, 20.39, 20.51, 20.90, 49.10, 60.32, 67.05, 67.70, 69.09, 70.40, 99.95, 102.25, 115.09, 117.40, 131.52, 134.99, 139.45, 141.57, 151.44, 161.86, 168.44, 169.25, 169.47, 169.97.

MS (ESI) m/z 520 (MH⁺).

compound 3: ¹H NMR (DMSO-d6): δ 1.20 (t, 6H), 1.99 (s, 3H), 2.03 (s, 3H), 2.09 (s, 3H), 4.15 (m, 4H), 4.31 (ddABX, 2H), 4.47 (m, 1H), 4.71 (s, 2H), 5.25 (m, 1H), 5.42 (m, 2H), 6.10 (s, 1H), 6.15 (s, 1H), 6.38 (m, 1H), 7.11 (s, 1H). ¹³C NMR (DMSO-d6): δ 13.87, 13.92, 20.41, 20.43, 20.51, 54.35, 60.39, 60.84, 66.72, 67.57, 68.28, 68.95, 99.99, 102.44, 113.82, 120.25, 132.31, 133.93, 139.47, 141.56, 151.93, 162.25, 168.25, 168.86, 169.43, 169.85.

MS (ESI) m/z 606 (MH⁺).

### Compound 4

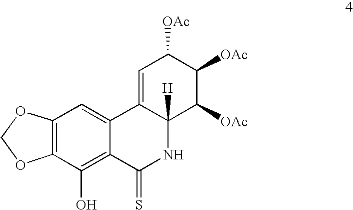

2,3,4,7-(tetraacetoxy)-narciclasine (150 mg, 316 μmol) and Lawesson's reagent (64 mg, 158 μmol) in anhydrous toluene (5 ml) were heated at 110° C. for 6 h. The reaction mixture was evaporated at reduced pressure and the crude product was further purified by column chromatography (cyclohexane/AcOEt 7:3 to 5:5). The product compound 4 was obtained as a yellow solid (33 mg, 23%) Rf 0.36 (cyclohexane/AcOEt 5:5).

¹H NMR (DMSO-d6): δ 1.99 (s, 3H), 2.02 (s, 3H), 2.10 (3H), 3.83 (m, 1H), 4.57 (m, 1H), 5.07 (dd, J 8.7, 2.4, 1H), 5.30 (m, 2H), 6.15 (s, 2H), 6.31 (m, 1H), 6.95 (s, 1H), 10.80 (s, 1H), 13.27 (s, 1H). ¹³C NMR (DMSO-d6): δ 19.85, 20.49, 20.50, 51.23, 66.31, 66.85, 68.29, 96.69, 102.02, 109.67, 118.48, 127.00, 132.56, 135.48, 144.42, 151.35, 168.68, 168.93, 169.32, 187.29.

MS (ESI) m/z 450 (MH⁺).

### Compound 5

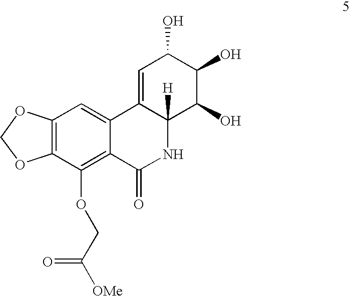

Compound 2 (111 mg, 0.214 mmole) and K₂CO₃ (3.5 mg, 0.009 mmole) were stirred at room temperature in methanol (10 ml) with one drop of water for 15 h. The reaction mixture was evaporated at reduced pressure and the crude product was further purified by column chromatography (CH₂Cl₂/MeOH 8:2). The product compound 5 was obtained as a white solid (62 mg, 74%) Rf 0.55 (CH₂Cl₂/MeOH 8:2).

¹H NMR (DMSO-d6): δ 3.68 (s, 3H), 3.79 (m, 1H), 4.03 (m, 2H), 4.74 (m, 2H), 4.96 (s, 1H), 5.19 (m, 2H), 6.04 (s, 1H), 6.11 (s, 2H), 7.02 (s, 1H), 7.22 (s, 1H). ¹³C NMR (DMSO-d6): δ 51.53, 52.54, 68.97, 69.07, 72.37, 99.70, 102.04, 114.81, 123.76, 131.08, 133.41, 138.68, 141.24, 151.32, 162.02, 168.96.

MS (ESI) m/z 380 (MH⁺).

### Compound 6

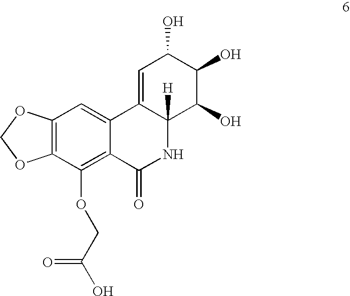

Compound 5 (50 mg, 0.132 mmole) was stirred at 0° C. in dioxane (3 ml) with 1 ml of water. A solution of LiOH 1M (8 eq) was added at 0° C. The reaction mixture was stirred at room temperature for 7 h and the dioxane was evaporated and LiOH was neutralized with citric acid 1M to obtain a pH=5. The solution was extracted with AcOEt and the organic phase was dried with Na₂SO₄ filtrated and evaporated. The crude product was washed with a minimum of AcOEt to obtain a white solid 6 (22 mg, 46%) Rf 0.13 (CH₂Cl₂/MeOH 8:2).

¹H NMR (DMSO-d6): δ 3.70 (s, 1H), 3.82 (s, 1H), 4.08 (m, 2H), 4.84 (s, 2H), 5.02 (s, 1H), 5.15 (s, 1H), 5.25 (s, 1H), 6.1 (s, 1H), 6.19 (m, 2H), 7.08 (s, 1H), 7.97 (s, 1H). ¹³C NMR (DMSO-d6): δ 52.66, 68.70, 68.96, 69.62, 72.34, 100.01, 102.56, 112.39, 124.80, 130.38, 133.70, 137.93, 141.14, 152.38, 170.15, 171.23.

MS (ESI) m/z 366 (MH⁺).

### Compound 7

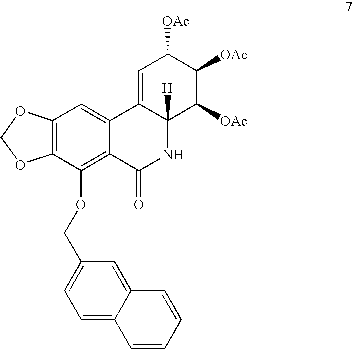

2,3,4-(triacetoxy)-narciclasine (70 mg, 161 μmol) and 2-(bromomethyl)naphtalene (36 mg, 162 μmol) were stirred at ambient temperature in acetonitrile then K₂CO₃ (22 mg, 160 μmol) was added. The reaction mixture was then heated to 60° C. for 22 h and then this mixture was evaporated. Chromatography (cyclohexane/AcOEt 7:3) gave 7 (70 mg, 76%) Rf 0.44 (cyclohexane/AcOEt 1:1).

¹H NMR (DMSO-d6): δ 2.01 (s, 3H), 2.07 (s, 3H), 2.12 (s, 3H), 4.43 (m, 1H), 5.04 (m, 1H), 5.32 (m, 4H), 6.11 (s, 1H), 6.17 (s, 1H), 6.30 (m, 1H), 7.14 (s, 1H), 7.54 (m, 1H), 7.93 (m, 3H), 8.05 (s, 1H), 8.36 (s, 1H).

MS (ESI) m/z 574 (MH⁺).

### Compound 8

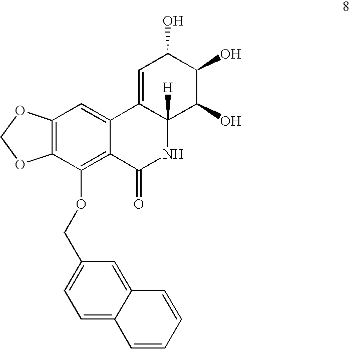

Compound 7 (70 mg, 0.122 mmole) and K₂CO₃ (3.5 mg, 0.025 mmole) were stirred at room temperature in methanol (5 ml) and CH₂Cl₂ (2 ml) with one drop of water for 15 h. The reaction mixture was evaporated at reduced pressure and the crude product was further purified by column chromatography (CH₂Cl₂/MeOH 8:2). The product was obtained as a white solid 8 (44 mg, 81%) Rf 0.62 (CH₂Cl₂/MeOH 8:2).

¹H NMR (DMSO-d6): δ 3.69 (m, 1H), 3.80 (m, 1H), 4.05 (m, 2H), 4.98 (m, 1H), 5.25 (m, 4H), 6.13 (m, 3H), 7.03 (s, 1H), 7.21 (s, 1H), 7.51 (m, 2H), 7.73 (m, 1H), 7.92 (m, 4H).

MS (ESI) m/z 448 (MH⁺).

### Compound 9

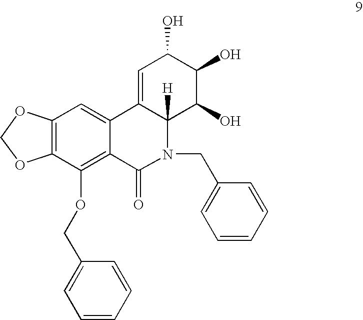

2,3,4-(triacetoxy)-narciclasine (100 mg, 230 μmol) and benzyl bromide (79 mg, 461 μmol) were stirred at ambient temperature in DMF then K₂CO₃ (48 mg, 350 μmol) was added. The reaction mixture was then heated to 60° C. for 22 h and then this mixture was evaporated and freeze-dried for 24 h. Chromatography (cyclohexane/AcOEt 7:3) gave oily product (56 mg, 39%) Rf 0.48 (cyclohexane/AcOEt 1:1). This product (50 mg, 0.081 mmole) and K₂CO₃ (3.5 mg, 0.009 mmole) were stirred at room temperature in methanol (5 ml) with one drop of water for 17 h. The reaction mixture was evaporated at reduced pressure and the crude product was further purified by column chromatography (CH₂Cl₂/MeOH 96:4 to 94:6). The product was obtained as a white solid 9 (30 mg, 74%) Rf 0.62 (CH₂Cl₂/MeOH 9:1).

¹H NMR (DMSO-d6): δ 3.58 (m, 1H), 4.07 (m, 2H), 4.16 (m, 1H), 4.94 (m, 2H), 5.12 (m, 4H), 6.15 (m, 3H), 6.99 (s, 1H), 7.31 (m, 11H).

¹³C NMR (DMSO-d6): δ 45.10, 54.78, 58.25, 66.64, 67.48, 72.81, 74.06, 99.48, 101.91, 115.87, 125.30, 125.96, 126.03, 127.64, 127.66, 127.99, 131.93, 133.90, 137.26, 139.33, 139.87, 141.84, 151.09, 162.44.

MS (ESI) m/z 488 (MH⁺).

### Compound 10

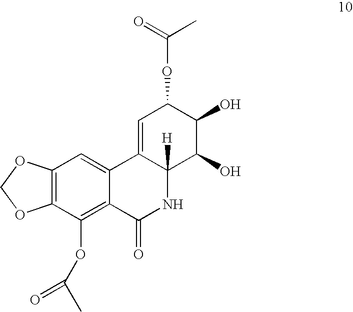

2,7-(diacetoxy)-3,4-(acetonide) narciclasine (98 mg, 227 μmol) was dissolved in 1 ml of THF and cooled to 0° C. A cooled solution of trifluoroacetic acid and water (2/1) was added at 0° C. The reaction mixture was stirred at 0° C. and then allowed to warm to room temperature in 1.4 h. The solvent was removed in vacuo. Chromatography (AcOEt) afforded 10 (52 mg, 59%).

¹H NMR (DMSO-d6): δ 2.06 (s, 3H), 2.27 (s, 3H), 3.70-3.78 (m, 2H), 4.16 (d, J 7.5, 1H), 5.25 (t, J 3.6, 1H), 5.38 (d, J 5.4, 1H), 5.50 (d, J 3.9, 1H), 6.16 (m, 1H), 6.20 (s, 2H), 7.26 (s, 1H), 7.38 (s, 1H).

MS (ESI) m/z 392 (MH⁺).

### Compound 11

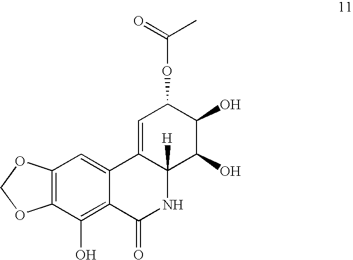

Compound 10 (46 mg, 118 μmol) was dissolved in 2 ml of pyridine and 1 ml of water and heated to reflux for 2 h. The solvent was removed in vacuo and the residue was freeze-dried for 48 h. Chromatography (Cyclohexane/AcOEt 100:0 to 0:100) afforded 11 (27 mg, 66%). Rf 0.49 (AcOEt).

¹H NMR (DMSO-d6): δ 2.06 (s, 3H), 3.75 (dd, J 2.1, 8.4, 1H), 3.80 (s, 1H), 4.28 (d, J 8.4, 1H), 5.23 (m, 1H), 5.65 (br, 2H), 6.12 (s, 1H), 6.16 (m, 2H), 6.92 (s, 1H), 8.09 (s, 1H), 13.24 (s, 1H).

MS (ESI) m/z 350 (MH⁺).

### Compound 12

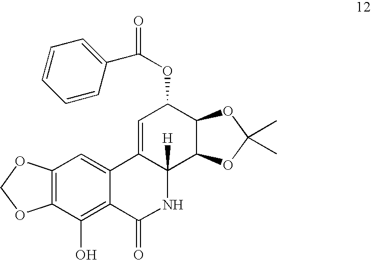

Benzoyl chloride (43 μl, 1 eq) was added to 3,4-(acetonide)-narciclasine (117 mg, 337 μmol) in 7 ml of dry pyridine under argon. The reaction mixture was stirred at room temperature for 1 day and two portions of benzoyl chloride (22 μl and 10 μl) were further added for complete consumption of starting material. The solvent was evaporated in vacuo and freeze-dried overnight. Chromatography (Cyclohexane/AcOEt 70:30) afforded compound 12 (80 mg, 53%). Rf 0.61 (cyclohexane/AcOEt 1:1).

¹H NMR (DMSO-d6): δ 1.38 (s, 3H), 1.51 (s, 3H), 4.32 (d, J 7.8, 1H), 4.41-4.50 (m, 2H), 5.60 (m, 1H), 6.10 (m, 2H), 6.71 (m, 1H), 7.14 (s, 1H), 7.58-7.65 (m, 3H), 7.97 (m, 2H), 9.02 (s, 1H), 13.75 (s, 1H).

MS (ESI) m/z 452 (MH⁺)

### Compound 13

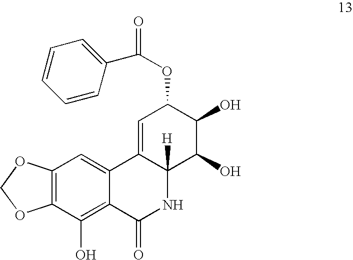

3 ml of a cooled solution of trifluoroacetic acid and water (2/1) is added at 0° C. to a mixture of compound 12 (93 mg, 206 μmol) in 6 ml of THF. After 2.5 h, the reaction mixture is evaporated to dryness. Chromatography (Cyclohexane/AcOEt 100:0 to 30:0) afforded 13 (50 mg, 60%). Rf 0.59 (AcOEt).

¹H NMR (DMSO-d6): δ 3.90-3.95 (m, 2H), 4.36 (d, J 8.1, 1H), 5.44 (br s, 1H), 5.52 (m, 1H), 5.7 (br s, 1H), 6.12 (m, 2H), 6.30 (m, 1H), 6.96 (s, 1H), 7.51-7.59 (m, 2H), 7.65-7.72 (m, 1H), 7.97-8.01 (m, 2H), 8.14 (s, 1H), 13.27 (s, 1H).

¹³C NMR (DMSO-d6): δ 53.33, 69.79, 69.96, 72.24, 96.79, 102.82, 106.38, 119.58, 129.12, 129.37, 129.91, 131.74, 134.13, 134.38, 134.57, 145.45, 152.96, 165.46, 169.47.

MS (ESI) m/z 412 (MH⁺).

### Compound 14 and 15

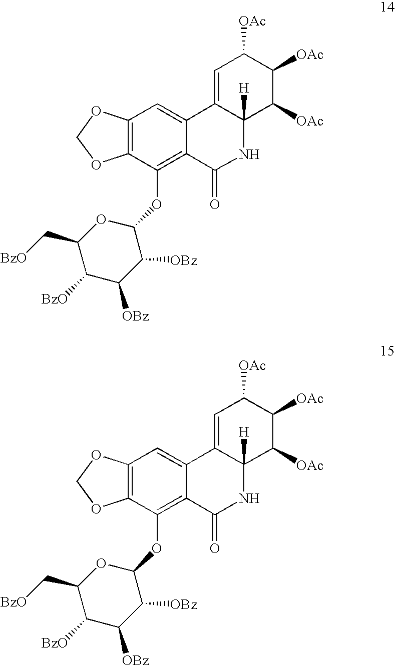

10 ml of dry dichloromethane was added to a mixture of 1-bromo-perbenzoylated α-glucose (455 mg, 0.69 mmol) and 2,3,4-(triacetoxy)-narciclasine (150 mg, 0.35 mmol). The mixture was cooled to ±78° C. and allyltrimethylsilane (2 mmol) was added at once to a solution of silver trifluoromethane sulfonate (0.69 mmol) in 5 ml of dry toluene. We then let the temperature return to room temperature without removing the cooling bath. The mixture was filtrated on celite and evaporated. Purification of the crude mixture by silica gel chromatography (cyclohexane/ethyl acetate 7:3 to 5:5) gave the α-glucosylated derivative 14 (Rf 0.53 (CH₂Cl₂/MeOH 96:4), 143 mg, 41%) and the β-glucosylated derivative 15 (Rf 0.38 (CH₂Cl₂/MeOH 96:4), 92 mg, 26%).

### Compound 14:

¹H NMR (DMSO-d6): δ 2.02 (s, 3H), 2.05 (s, 3H), 2.09 (s, 3H), 4.42 (m, 3H), 4.99 (d, J 9.0, 1H), 5.30 (m, 3H), 5.65 (dd, J 3.9 and 9.9, 1H), 5.70 (dd, J 9.9 and 9.9, 1H), 6.03 (s, 1H), 6.10 (s, 1H), 6.26 (m, 1H), 6.45 (d, J 3.3, 1H), 6.56 (dd, J 9.6 and 9.6, 1H), 7.09 (s, 1H), 7.41-7.90 (m, 20H), 8.54 (s, 1H)

MS (ESI) m/z 1012 (MH+), 1034 (MNa⁺).

### Compound 15:

¹H NMR (DMSO-d6): δ 1.97 (s, 3H), 2.01 (s, 3H), 2.10 (s, 3H), 4.36 (m, 1H), 4.49 (m, 3H), 4.92 (dd, J 1.8 and 8.7, 1H), 5.26 (m, 2H), 5.62 (m, 2H), 6.06 (d, J 7.5, 1H), 6.00 (s, 1H), 6.03 (s, 1H), 6.17 (m, 1H), 7.01 (s, 1H), 7.40-7.90 (m, 20H), 8.22 (s, 1H)

MS (ESI) m/z 1012 (MH+), 1034 (MNa⁺).

### Compound 16:

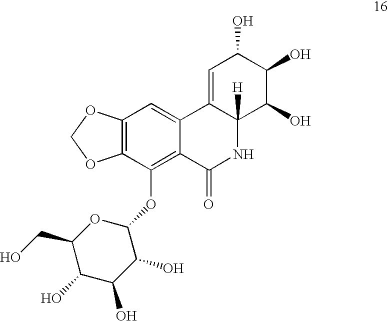

Compound 14 (113 mg, 0.112 mmole) and K₂CO₃ (20 mg, 0.144 mmole) were stirred at room temperature in methanol (15 ml) with few drops of water for 16 h. The reaction mixture was evaporated at reduced pressure and the crude product was further purified by column chromatography (CH₂Cl₂/MeOH/H₂O 70:30:5). The product was obtained as a white solid 16 (44 mg, 84%) Rf 0.60 (CH₂Cl₂/MeOH H₂O 6:4:few drops of water).

¹H NMR (DMSO-d6): δ 3.20-4.11 (m, 17H), 5.25 (d, J 3.6, 1H), 6.08 (s, 1H), 6.11 (s, 1H), 6.17 (m, 1H), 7.13 (s, 1H), 7.72 (s, 1H)

¹³C NMR (DMSO-d6): δ 52.70, 60.16, 68.80, 68.92, 69.05, 71.70, 72.34, 73.77, 74.04, 100.50, 102.24, 103.03, 114.34, 124.25, 130.69, 133.93, 140.12, 141.36, 151.42, 163.36.

MS (ESI) m/z 492 (MNa⁺).

### Compound 17

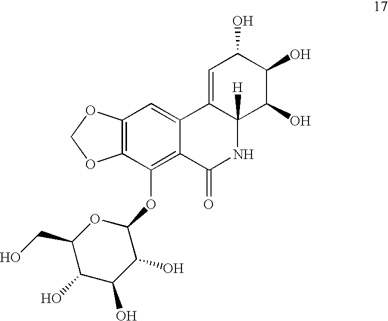

Compound 15 (55 mg, 0.05 mmole) and K₂CO₃ (10 mg, 0.072 mmole) were stirred at room temperature in methanol (7 ml) with few drops of water for 17 h. The reaction mixture was evaporated at reduced pressure and the crude product was further purified by column chromatography (CH₂Cl₂/MeOH/H₂O 70:30:5). The product was obtained as a white solid 17 (24 mg, 94%) Rf 0.65 (CH₂Cl₂/MeOH H₂O 6:4:few drops of water).

¹H NMR (DMSO-d6): δ 3.20-4.03 (m, 11H), 4.48 (m, 1H), 4.80 (d, J 6.6, 1H), 5.06 (m, 3H), 5.23 (m, 1H), 6.06 (s, 1H), 6.14 (s, 1H), 6.15 (m, 1H), 7.05 (s, 1H), 7.79 (s, 1H)

MS (ESI) m/z 492 (MNa⁺).

### Compound 18:

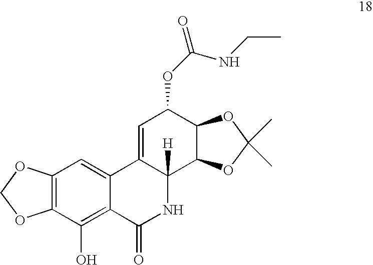

Ethyl isocyanate (40 μl, 4 eq) was added to 3,4-(acetonide)-narciclasine (30 mg, 0.078 mmol) in 2 ml of dry DMF under argon. The reaction mixture was stirred at room temperature for 1 day and ethyl isocyanate (40 μl, 4 eq) were further added for complete consumption of starting material. The solvent was evaporated in vacuo. Chromatography (Cyclohexane/AcOEt 6:4 to 7:3) afforded compound 18 (20 mg, 62%). Rf 0.43 (cyclohexane/AcOEt 1:1).

¹H NMR (DMSO-d6): δ 1.08 (m, 3H), 1.35 (s, 3H), 1.49 (s, 3H), 3.10 (m, 2H), 4.21 (m, 3H), 4.18 (m, 1H), 6.09 (s, 1H), 6.11 (s, 1H), 6.51 (m, 1H), 7.14 (s, 1H), 7.41 (m, 1H), 8.91 (s, 1H), 13.75 (s, 1H).

MS (ESI) m/z 419 (MH⁺)

### Compound 19:

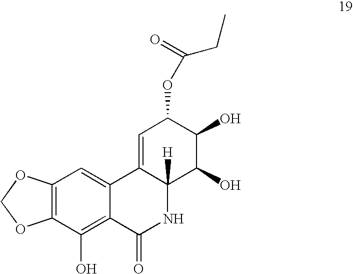

Propionic anhydride (1 ml, 7.7 mmol) was added to 3,4-(acetonide)-narciclasine (150 mg, 0.432 mmol) in 5 ml of dry pyridine under argon. The reaction mixture was stirred at 60° C. for 17 h. The solvent was evaporated in vacuo and freeze-dried overnight. The residue was dissolved with 1 ml of pyridine and 0.5 ml of water and the solution was heated to reflux for 1.5 h. The solution was evaporated to dryness. Chromatography (Cyclohexane/AcOEt 6:4 to 4:6) afforded 158 mg of the 3,4-(acetonide) derivative of 19 (91%) Rf 0.51 (cyclohexane/AcOEt 1:1). This later compound (170 mg, 0.41 mmol) was dissolved with 2 ml of THF and the mixture was cooled at 0° C. and 6 ml of a solution CF₃COOH/H₂O (2:1) was added. The mixture was stirred at ambient temperature for 1.5 h and the solvent was evaporated and freeze-dried overnight. Chromatography (Cyclohexane/AcOEt 1:1 to 1:9) afforded compound 19 (63 mg, 40%) Rf 0.20 (AcOEt).

¹H NMR (DMSO-d6): δ 1.02 (t, J 7.5, 3H), 2.32 (q, J 7.2, 2H), 3.73 (m, 2H), 4.26 (m, 1H), 5.23 (m, 1H), 5.35 (d, J 5.1, 1H), 5.51 (d, J 3.9, 1H), 6.11 (m, 3H), 6.89 (s, 1H), 8.05 (s, 1H), 13.21 (s, 1H).

¹³C NMR (DMSO-d6): δ 8.88, 26.76, 52.63, 69.06, 69.19, 70.78, 96.07, 102.14, 105.66, 119.23, 131.13, 133.19, 133.84, 144.81, 152.31, 168.77, 172.82.

MS (ESI) m/z 364 (MH⁺)

### Compound 20:

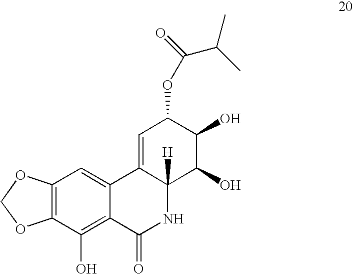

Isobutyric anhydride (1 ml, 6 mmol) was added to 3,4-(acetonide)-narciclasine (110 mg, 0.317 mmol) in 4 ml of dry pyridine under argon. The reaction mixture was stirred at 60° C. for 18 h. The solvent was evaporated in vacuo and freeze-dried overnight. The residue was dissolved with 4 ml of pyridine and 2 ml of water and the solution was heated to reflux for 4 h. The solution was evaporated to dryness. The residue was dissolved with 2 ml of THF and the mixture was cooled at 0° C. and 6 ml of a solution CF₃COOH/H₂O (2:1) was added. The mixture was stirred at ambient temperature for 2 h and the solvent was evaporated and freeze-dried overnight. Chromatography (Cyclohexane/AcOEt 1:1 to 3:7) afforded compound 20 (73 mg, 61%) Rf 0.5 (AcOEt).

¹H NMR (DMSO-d6): δ 1.07 (d, J 6.6, 3H), 1.09 (d, J 6.6, 3H), 2.53 (m, 1H), 3.73 (m, 2H), 4.26 (m, 1H), 5.22 (m, 1H), 5.40 (m, 2H), 6.11 (m, 3H), 6.90 (s, 1H), 8.05 (s, 1H), 13.22 (s, 1H).

¹³C NMR (DMSO-d6): δ 18.55, 18.70, 33.08, 52.65, 69.08, 69.20, 70.69, 96.07, 102.13, 105.65, 119.06, 131.10, 133.26, 133.84, 144.78, 152.30, 168.77, 175.25.

MS (ESI) m/z 400 (MH⁺)

### Compound 21:

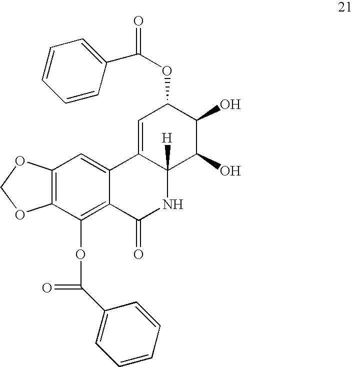

Benzoyl chloride (49 μl, 1 eq) was added to 3,4-(acetonide)-narciclasine (150 mg, 0.432 mmol) in 7 ml of dry pyridine under argon. The reaction mixture was stirred at room temperature for 17 h and one portion of benzoyl chloride (49 μl) were further added for complete consumption of starting material. The solvent was evaporated in vacuo and freeze-dried overnight. Chromatography (Cyclohexane/AcOEt 70:30) afforded 122 mg of the 3,4-(acetonide) derivative of 21 (51%) Rf 0.32 (cyclohexane/AcOEt 1:1). This later compound (122 mg, 0.22 mmol) was dissolved with 2 ml of THF and the mixture was cooled at 0° C. and 6 ml of a solution CF₃COOH/H₂O (2:1) was added. The mixture was stirred at ambient temperature for 4 h and the solvent was evaporated and freeze-dried overnight. Chromatography (Cyclohexane/AcOEt 1:1 to 3:7) afforded compound 21 (59 mg, 52%) Rf 0.54 (AcOEt).

¹H NMR (DMSO-d6): δ 3.90 (m, 2H), 4.25 (m, 1H), 5.41 (d, J 6.0, 1H), 5.54 (m, 1H), 5.61 (d, J 4.2, 1H), 6.20 (s, 2H), 6.32 (m, 1H), 7.32 (s, 1H), 7.34 (s, 1H), 7.50-7.77 (m, 6H), 7.98 (m, 2H), 8.10 (m, 2H).

¹³C NMR (DMSO-d6): δ 52.54, 69.17, 69.57, 71.59, 102.13, 103.21, 114.87, 119.11, 128.73, 128.74, 128.80, 129.26, 129.31, 129.88, 132.16, 132.70, 133.49, 133.79, 134.63, 140.77, 151.64, 161.42, 163.69, 164.84.

MS (ESI) m/z 516 (MH⁺), 538 (MNa⁺).

### Compound 22:

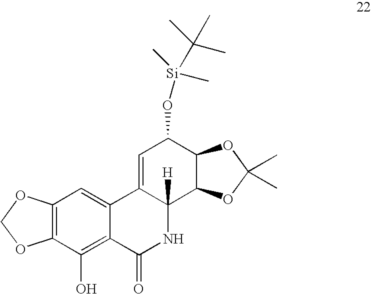

tert-Butylchlorodimethylsilane (547 mg, 3.63 mmol) was added to 3,4-(acetonide)-narciclasine (420 mg, 1.21 mmol) in 30 ml of dry DMF under argon. The reaction mixture was stirred at room temperature for 17 h. The solvent was evaporated in vacuo and freeze-dried overnight. Chromatography (Cyclohexane/AcOEt 9/1) afforded compound 22 (330 mg, 59%). Rf 0.67 (cyclohexane/AcOEt 1:1).

¹H NMR (DMSO-d6): δ 0.13 (s, 3H), 0.15 (s, 3H), 0.93 (s, 9H), 1.32 (s, 3H), 1.46 (s, 3H), 3.95 (dd, J 6.6, 1H), 4.12 (dd, J 8.1 and 7.2, 1H), 4.20 (m, 1H), 4.31 (m, 1H), 6.08 (s, 2H), 6.43 (m, 1H), 7.11 (s, 1H), 8.87 (s, 1H), 13.74 (s, 1H).

¹³C NMR (DMSO-d6): δ −4.98, −4.66, 17.86, 24.80, 25.74, 26.93, 54.47, 73.19, 78.23, 78.79, 94.39, 102.00, 104.25, 109.95, 126.28, 127.47, 128.54, 133.37, 145.12, 152.48, 167.50.

MS (ESI) m/z 462 (MH⁺), 484 (MNa⁺).

### Compound 23:

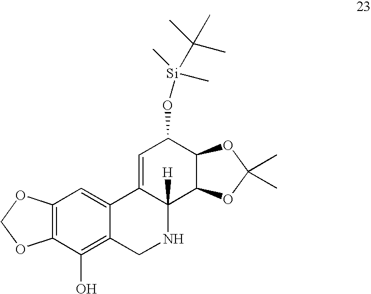

Lithium aluminium hydride (102 mg, 2.68 mmol) was added to compound 22 (300 mg, 0.671 mmol) in 13 ml of dry THF under argon. The reaction mixture was stirred and heated at reflux for 3 h. Then 3 ml of water was added and the solvent was evaporated in vacuo and freeze-dried overnight. Chromatography (CH₂Cl₂/MeOH 9/1) afforded compound 23 (170 mg, 76%). Rf 0.53 (CH₂Cl₂/MeOH 9/1).

¹H NMR (DMSO-d6): δ 0.11 (s, 3H), 0.12 (s, 3H), 0.92 (s, 9H), 1.29 (s, 3H), 1.42 (s, 3H), 3.40 (m, 1H), 3.98 (m, 3H), 4.25 (m, 1H), 5.89 (m, 1H), 5.93 (s, 2H), 6.77 (s, 1H), 9.44 (m, 1H).

MS (ESI) m/z 448 (MH⁺).

### Compound 24:

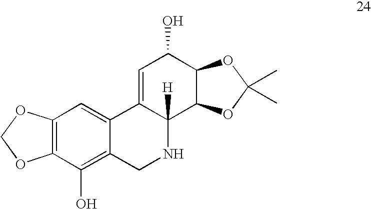

tetrabutylammonium fluoride (1.34 ml of 1M in THF, 2 eq) was added to compound 23 (300 mg, 0.67 mmol) in 10 ml of dry THF under argon. The reaction mixture was stirred at ambient temperature for 18 h. The solvent was evaporated in vacuo and freeze-dried overnight. Chromatography (CH₂Cl₂/MeOH 95/5) afforded compound 24 (157 mg, 70%). Rf 0.35 (CH₂Cl₂/MeOH 9/1).

¹H NMR (DMSO-d6): δ 1.30 (s, 3H), 1.42 (s, 3H), 3.24 (m, 1H), 3.40 (m, 1H), 3.98 (m, 3H), 4.12 (m, 1H), 5.54 (d, J 5.4, 1H), 5.93 (s, 2H), 5.95 (m, 1H), 6.72 (s, 1H), 9.43 (m, 1H).

¹³C NMR (DMSO-d6): δ 24.55, 27.07, 41.22, 57.63, 70.67, 77.65, 80.39, 95.16, 100.31, 108.42, 121.47, 123.01, 125.30, 131.79, 133.50, 135.99, 146.74.

MS (ESI) m/z 334 (MH⁺).

### Compound 25:

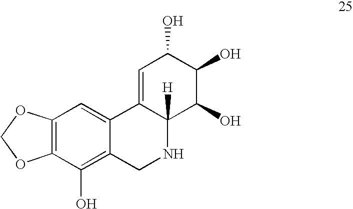

Eight drops of water and four drops of sulfuric acid (98%) were added to compound 24 (130 mg, 0.39 mmol) in 9 ml of THF and 9 ml of CH₂Cl₂. The reaction mixture was stirred at ambient temperature for 20 min. The solution was neutralized with a solution of NaHCO₃ saturated. The solvent was evaporated in vacuo and freeze-dried overnight. Chromatography (CH₂Cl₂/MeOH 8/2 to 5/5) afforded compound 25 (89 mg, 78%). Rf 0.16 (CH₂Cl₂/MeOH 7/3).

¹H NMR (DMSO-d6): δ 3.15 (m, 1H), 3.61 (m, 3H), 3.98 (m, 2H), 4.90 (m, 2H), 5.93 (m, 3H), 6.79 (s, 1H).

¹³C NMR (DMSO-d6): δ 43.46, 55.34, 69.46, 70.09, 73.00, 95.07, 100.30, 116.71, 120.75, 126.11, 133.35, 136.18, 146.61.

MS (ESI) m/z 294 (MH⁺).

### Compound 26:

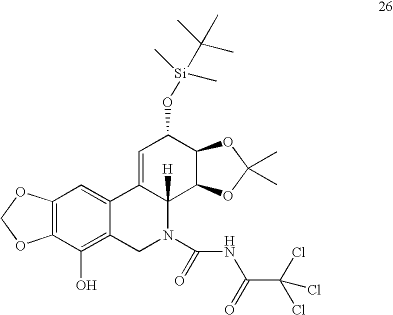

Trichloroacetyl isocyanate (0.16 ml, 1.35 mmol) was added to compound 23 (320 mg, 0.71 mmol) in 40 ml of 2-butanone. The reaction mixture was stirred at 0° C. for 30 min. Then 2 drops of water was added and the solvent was evaporated in vacuo. Chromatography (CH₂Cl₂/MeOH 96/4) afforded compound 26 (719 mg, 100%). Rf 0.75 (CH₂Cl₂/MeOH 9/1).

¹H NMR (DMSO-d6): δ 0.13 (s, 3H), 0.14 (s, 3H), 0.92 (s, 9H), 1.29 (s, 3H), 1.40 (s, 3H), 3.75 (m, 1H), 3.98 (m, 1H), 4.52 (m, 2H), 5.98 (m, 3H), 6.77 (s, 1H), 8.30 (m, 2H), 9.90 (m, 1H).

MS (ESI) m/z 635 (MH⁺).

### Compound 27:

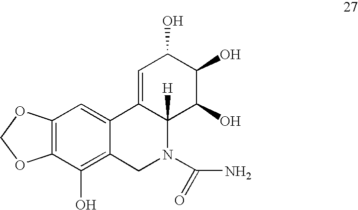

Tetrabutylammonium fluoride (1.31 ml of 1M in THF, 4.42 mmol) was added to compound 26 (700 mg, 1.10 mmol) in 20 ml of dry THF under argon. The reaction mixture was stirred at ambient temperature for 17 h and heated at 50° C. for 3 h. The solvent was evaporated in vacuo. The residue was then dissolved in 10 ml of methanol and 10 ml of K₂CO₃ (5%) was then added. The solution was stirred at 50° C. for 17 h. The solvent was evaporated in vacuo and freeze-dried overnight. The residue was dissolved in 6 ml of THF and 9.6 ml of trifluoroacetic acid with 4.8 ml of water was added. The solution was stirred at 0° C. for 1 h and then freeze-dried overnight. Chromatography (CH₂Cl₂/MeOH 95/5) afforded compound 27 (132 mg, %). Rf 0.25 (CH₂Cl₂/MeOH 9/1).

¹H NMR (DMSO-d6): δ 3.10 (m, 1H), 3.77 (m, 1H), 3.88 (m, 1H), 4.01 (m, 1H), 4.50 (m, 1H), 5.04 (m, 2H), 5.30 (m, 1H), 5.62 (m, 1H), 5.85 (m, 1H), 5.94 (s, 1H), 5.94 (s, 1H), 6.49 (s, 1H), 9.56 (s, 1H).

¹¹³C NMR (DMSO-d6): δ 35.81, 54.73, 67.44, 69.41, 73.69, 97.26, 100.39, 124.08, 133.87, 134.45, 135.78, 146.79, 160.20.

MS (ESI) m/z 337 (MH⁺).

### Compound 266:

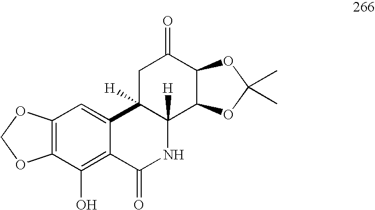

Narciclasine (500 mg, 1.63 mmol) was dissolved in 75 ml of ethanol and 75 ml of CH₂Cl₂. At this solution, 500 mg of Pd/C 10% was added. Then triethylamine (0.996 ml, 7.17 mmol) and formic acid (0.27 ml, 7.17 mmol) were added. This solution was stirred and heated to 80° C. for 6 h. The reaction mixture was filtered and the solvent was removed in vacuo. The residue was dissolved in 20 ml of water and extracted with ethyl acetate (4×200 ml). The organic phase was dried with Na₂SO₄ and evaporated to dryness. This residue was dissolved in DMF, paratoluenesulfonic acid (0.18 g, 0.94 mmol) and 2,2-dimethoxypropane (2.24 ml, 1.90 mmol) were added. The solution was stirred at ambient temperature for 20 h. The solvent was evaporated in vacuo and freeze-dried overnight. Chromatography (cyclohexane/AcOEt 1/1) gave 125 mg of the 2-hydroxy-derivative of 266 (Rf 0.23 (cyclohexane/AcOEt 1/1)) and 99 mg of the cis derivative. A solution of 2 ml of dry THF with DMSO (92 μl, 1.29 mmol) and oxalyl chloride (76 μl, 0899 mmol) was prepared at −80° C. under argon atmosphere and a solution of 2-hydroxy derivative of 266 (205 mg, 0.59 mmole) in 12 ml of THF was added after 5 min. After 10 min, triethylamine (408 μl, 2.93 mmol) was added to the reaction mixture. We then let the temperature return to room temperature. The reaction mixture was evaporated to dryness and chromatography (cyclohexane/AcOEt 1) was performed to obtain compound 266 (155 mg, 31%) Rf 0.40 (cyclohexane/AcOEt 1/2).

¹H NMR (DMSO-d6): δ 1.37 (s, 3H), 1.39 (s, 3H), 2.35 (m, 1H), 3.53 (m, 2H), 4.65 (m, 1H), 4.86 (d, J 8.1, 1H), 6.07 (s, 1H), 6.09 (s, 1H), 6.52 (s, 1H), 8.94 (s, 1H), 13.23 (s, 1H).

¹¹³C NMR (DMSO-d6): δ 25.48, 26.99, 31.77, 40.13, 56.19, 77.56, 78.50, 97.84, 101.85, 105.50, 110.34, 135.76, 145.30, 152.24, 168.96, 205.47.

MS (ESI) m/z 350 (MH⁺).

### Compound 260 or 270:

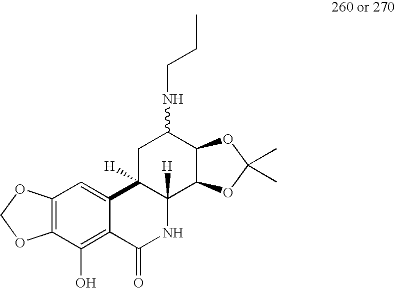

Compound 266 (150 mg, 0.432 mmol) was dissolved in 8 ml of dichloroethane. Few balls of 4A molecular sieves, propylamine (53 μl, 0.648 mmol) then sodium triacetoxyborohydride (145 mg, 0.692 mmol) were added. The solution was stirred at ambient temperature for 17 h. The reaction mixture was evaporated to dryness and chromatography (AcOEt/MeOH 9/1 to 8/2) was performed to obtain compound 260 or 270 (108 mg, 67%) Rf 0.23 (AcOEt/MeOH 9/1).

¹H NMR (DMSO-d6): δ 0.92 (t, J 7.2, 3H), 1.21 (s, 3H), 1.35 (s, 3H), 1.43 (s, 3H), 1.46 (m 2H), 2.41 (m, 1H), 2.65 (m, 2H), 3.02 (m, 1H), 3.25 (m, 1H), 4.11 (m, 1H), 4.41 (dd, J 3.9, 1H), 6.05 (s, 1H), 6.08 (s, 1H), 6.63 (s, 1H), 8.63 (s, 1H), 13.20 (s, 1H).

MS (ESI) m/z 391 (MH⁺).

### Compound 261 or 271:

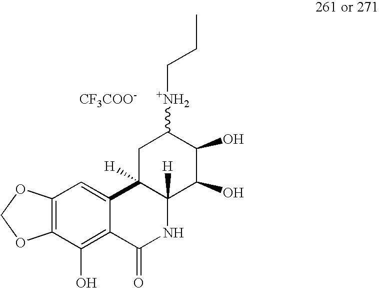

3 ml of a cooled solution of trifluoroacetic acid and water (2/1) is added at 0° C. to a mixture of compound 260 or 270 (106 mg, 0.272 mmol) in 5 ml of THF. After 10 h, the reaction mixture is evaporated in vacuo and freeze-dried overnight. Chromatography (CH₂Cl₂/MeOH 9:1) afforded compound 261 or 271 (128 mg, 60 100%). Rf 0.59 (AcOEt).

¹H NMR (DMSO-d6): δ 0.94 (s, 3H), 1.50 (m 3H), 2.41 (m, 1H), 2.66-2.99 (m, 3H), 3.22 (m, 1H), 3.56 (m, 1H), 4.06 (m, 1H), 5.41 (m, 1H), 6.08 (s, 1H), 6.09 (s, 1H), 6.60 (s, 1H), 7.77 (s, 1H), 13.02 (s, 1H).

MS (ESI) m/z 351 (MH⁺).

Exemplary compounds set out in tabulated form in Tables 1, 2, 3, 4, 5, and 6 are synthesized using similar protocols.

### Example 3

### In Vitro and In Vivo Pharmacological Evaluation (Maximum Tolerated Dose)

### a) In Vitro

MTT tests were performed in order to rapidly, i.e. within 5 days, measure the effect of compounds of this invention on the overall cell growth. The test measured the number of metabolically active living cells that were able to transform the yellow product 3-(4,5-dimethylthiazol-2-yl)-2,5-diphenyl tetrazolium bromide (herein referred as MTT) into the blue product formazan dye by mitochondrial reduction. The amount of formazan obtained at the end of the experiment, measured by means of a spectrophotometer, is directly proportional to the number of living cells. Optical density determination thus enabled a quantitative measurement of the effect of the investigated compounds as compared to the control condition (untreated cells) and/or to other reference compounds such as narciclasine.

Six human cancer cell lines described in table 7 were used in the following MTT tests. These cancer cell lines cover six histological cancer types, being prostate, glioma, pancreas, colon, lung, and breast cancers.

To perform the assay, cells were allowed to grow in 96-well micro-wells with a flat bottom with an amount of 100 μl of cell suspension per well with 1,000 to 4,000 cells/well depending on the cell type used. Each cell line was seeded in a well known MEM 10% serum culture medium.

TABLE 7

| | ATCC | | |
|---|---|---|---|
| Tumor cell lines | code | Tissue | Literature reference |
| PC3 | CRL-1435 | Prostate | Invest. Urol. 17: 16-23, 1979; Cancer Res. 40: 524-534, |
| | | | 1980 |
| U-373MG | HTB-17 | Glioma | Acta Pathol. Microbial. Scand. 74: 465-486, 1968 |
| BxPC3 | CRL-1687 | Pancreas | Cancer Invest. 4: 15-23, 1986; Clin. Lap. Med. 2: |
| | | | 567-578, 1982 |
| LoVo | CCL-229 | Colon | Exp. Cell Res. 101: 414-416, 1976; J. Natl. Cancer |
| | | | Inst. 61: 75-83, 1978; Cancer Res. 39: 2630-2636, |
| | | | 1979 |
| A549 | CCL-185 | Lung | J. Natl. Cancer Inst. 51: 1417-1423, 1973; Int. J. |
| | | | Cancer 17: 62-70, 1976 |
| MCF-7 | HTB-22 | Breast | J. Natl. Cancer Inst. 51: 1409-1416, 1973 |

The detailed experimental procedure was the following: after a 24-hour period of incubation at 37° C., the culture medium was replaced by 100 μl of fresh medium in which the tested compound was previously dissolved, at the following molar concentrations: 10⁻⁹ M, 5.10⁻⁹ M, 10⁻⁹ M, 5.10⁻⁹ M, 10⁻⁷ M, 5.10⁻⁷ M, 10⁻⁶ M, 5.10⁻⁶ M, and 10⁻⁶ M. Each experiment was repeated 6 times.

After 72 hours of incubation at 37° C. with (experimental conditions) or without (control condition) the compound to be tested, the medium was replaced by 100 μl MTT dissolved in RPMI (1640 without phenol red) at a concentration of 1 mg/ml. The micro-wells were subsequently incubated during 3 hours at 37° C. and centrifuged at 400 g during 10 minutes. MTT was removed and formazan crystals formed were dissolved in 100 μl DMSO. The micro-wells were shaken for 5 minutes and read on a spectrophotometer at wavelengths of 570 nm (maximum formazan absorbance) and 630 nm (background noise).

For each experimental condition, the mean optical density was calculated, allowing the determination of the percentage of remaining living cells in comparison to the control.

Table 8 shows for compounds according to the invention, and a reference compound narciclasine, the IC₅₀ obtained for the human cancer cell lines (mean of the six cell lines). The IC₅₀ represents the range of molar concentrations of the compound tested that resulted in a 50% inhibition of overall tumor cells growth.

### b) In Vivo

The maximum tolerated dose (herein after MTD) is defined as the maximum amount of a given drug which can be administered acutely (i.e. in one intraperitoneal, intravenous, subcutaneous or oral single dose) to healthy animals, i.e. animals not grafted with tumors. In this assay, compounds were administered intravenously. The survival times and weights of the animals were recorded up to 14 days post injection. Five different doses of each compound were used for the determination of the MTD index. Each experimental group comprised 3 mice for the determination of the MTD (expressed in mg/kg). Using this methodology, the following data were obtained and reported in table 8:

TABLE 8
IC₅₀ and MTD of compounds according to the invention

| compound | IC₅₀ (M) | MTD (mg/kg) |
|---|---|---|
| narciclasine | 5.10⁻⁸-10⁻⁸ | 10 |
| 1 | 5.10⁻⁶-10⁻⁶ | >80 |
| 2 | >10⁻⁵ | nd |
| 3 | >10⁻⁵ | nd |
| 4 | 5.10⁻⁶-10⁻⁶ | 40 |
| 5 | 10⁻⁵-5.10⁻⁶ | nd |
| 6 | 10⁻⁵-5.10⁻⁶ | nd |
| 7 | 5.10⁻⁶-10⁻⁶ | nd |
| 8 | 5.10⁻⁶-10⁻⁶ | >80 |
| 9 | >10⁻⁵ | 40 |
| 10 | 5.10⁻⁷-10⁻⁷ | >80 |
| 11 | 5.10⁻⁷-10⁻⁷ | >80 |
| 13 | 5.10⁻⁸-10⁻⁸ | 10 |
| 19 | 5.10⁻⁸-10⁻⁸ | 40 |
| 20 | 5.10⁻⁸-10⁻⁸ | 40 |

It can be seen from Table 8, that compounds of the invention are particularly potent antiproliferative agents with IC₅₀ in the micromolar and sub-micromolar range. The compounds of the invention are particularly advantageous since they are as potent as narciclasine in vitro, while being similarly or significantly less toxic in vivo.

### Example 4

### Impairment of Cell Proliferation and Cell Migration Triggered by the Isocarbostyril Alkaloid Derivatives of the Present Invention in Cancer Cells

A cellular imaging approach (Debeir et al., Cytometry 60:29-40, 2004; Debeir et al., IEEE Trans Med Imaging 24:697-711, 2005) was used to investigate whether the isocarbostyril alkaloid derivatives of the present invention were able to impair cell proliferation and cell migration in apoptosis-sensitive (U-373MG—see Table 7) as well as apoptosis-resistant (PC3—see Table 7) cancer cells. Cellular imaging relied on the use of computer-assisted phase-contrast microscopy making possible to film the behavior of living cells in culture dishes for several days.

Cells were seeded in 25-cm² at a low density, treated or not with the compounds of the present invention (at a concentration of 100 nM) and filmed thereafter for a period of 72 h. The analysis of the films was performed by two operators, working independently. The effect on cell migration was characterized as +++ (high effect: cell migration of most cells is drastically altered for most of the duration of the film), ++ (medium effect: compound altering cell migration of an important proportion of the cells although less drastically), + (low effect: compound affecting cell migration of a small part of the cell population, or only for a limited part of the 72 h incubation), − (no effect: as compared to the control conditions, no difference is observed). The effect on the overall growth was measured by counting the number of cells on the first (Oh) and the last image (72 h) of each film. The global growth ratio (GGR) was then deduced by dividing the number of cells on the last image by the number of cells on the first image. The ratio GGR_{treated cells}/GGR_{control cells}, was further calculated thereby obtaining a value that describes the effect of compounds of the present invention on the overall cell growth. The ratio GGR_{treated cells}/GGR_{control cells} was measured for compounds 10, 11 and 13 and is reported in Table 9.

TABLE 9
Cellular imaging

| | Cell line | | | |
|---|---|---|---|---|
| | PC3 | | U-373 MG | |
| | | Ratio | | Ratio |
| compound | effect on cell | GGR_{treated cells}/ | effect on cell | GGR_{treated cells}/ |
| n^{o} | migration | GGR_{control cells} | migration | GGR_{control cells} |
| 10 | + | 1.38 | − | 1.06 |
| 11 | +++ | 0.40 | ++ | 0.42 |
| 13 | +++ | 0.31 | ++ | 0.45 |

The films (not shown) and data obtained clearly show that compounds of the present invention impair both cell proliferation and cell migration of human cancer cells.

1. A compound having the structural formula I or II, stereoisomers, tautomers, racemates, or a pharmaceutically acceptable salt and/or solvate thereof, 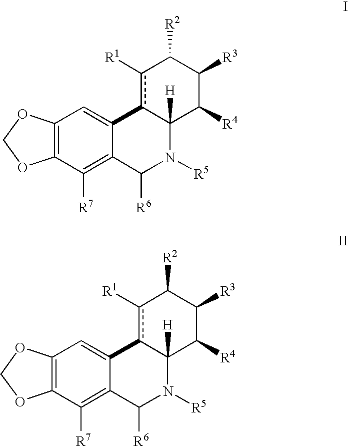
wherein
R¹ is hydrogen or a group selected from hydroxyl, alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, Het¹carbonyloxy, Het²-carbonyloxy, cycloalkylcarbonyloxy, aralkyl, cycloalkyl, Het¹, Het², alkenyl, alkynyl, alkylamino, arylamino, alkylaminoalkyl, alkylcarbonyl, alkylcarbonylamino, amino, cycloalkyloxy, alkylthio, arylthio, formylamino, Het¹alkyl, Het²alkyl, Het¹oxy, Het²oxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, alkenylamino, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkenylcarbonyloxy, Het¹amino, Het²-amino, Het¹carbonyloxy, Het²-carbonyloxy, cycloalkylcarbonyloxy, alkoxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, aryloxy, or saccharyl, each group being optionally substituted with one or more substituents independently selected from halogen, thiol, alkyl, haloalkyl, alkoxy, acyl, carboxyamino, acylamino, acetamido, haloalkoxy, alkylcarbonyl, aryl, aralkyl, Het¹, Het², cycloalkyl, carboxyl, oxo, cyano nitro, amino, aminoalkyl, alkylamino, alkenylamino, alkylamido, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, hydroxyalkyl, or phosphatyl,
R³ is a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²-amino, Het¹ carbonyloxy, Het²-carbonyloxy, cycloalkylcarbonyloxy, alkoxy, aryloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano nitro, aminoalkyl, alkylamino, alkenylamino, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
R⁴ is a group selected from hydroxyl, alkylcarbonyloxy, alkoxy, aryloxy, sulfato, arylalkyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²-amino, Het¹ carbonyloxy, Het²-carbonyloxy, cycloalkylcarbonyloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, alkyloxycarbonylalkyl, carboxyalkyl, alkylaminoalkyl, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, alkenylamino, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
or wherein R³ and R⁴ together with the carbon atoms to which they are attached form a dioxolane ring, said ring being optionally substituted with one or two substituents selected from alkyl or aryl,
R⁵ is hydrogen or a group selected from alkyl, alkyloxycarbonylalkyl, aryloxycarbonylalkyl, aryl, aralkyl, alkenyl, alkynyl, alkylaminoalkyl, alkylcarbonyl, arylcarbonyl, cycloalkyl, Het¹alkyl, Het²alkyl, alkyloxycarbonyl, aminocarbonyl, halo alkylcarbonylaminocarbonyl, alkylcarbonylaminocarbonyl, or cycloalkylalkyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, alkenylamino, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
R⁶ is hydrogen, oxo or thioxo,
R⁷ is hydrogen, hydroxyl or a group selected from alkoxy, alkenyloxy, alkynyloxy, aralkyloxy, alkylcarbonyloxy, arylcarbonyloxy, arylcarbonylalkyloxy, carboxyalkyloxy, alkylaminocarbonyloxy, arylaminocarbonyloxy, alkylaminothiocarbonyloxy, arylaminothiocarbonyloxy, alkyloxycarbonylalkyloxy, aryloxycarbonylalkyloxy, hydroxycarbonylalkyloxy, Het¹carbonyloxy, Het²-carbonyloxy, aryloxy, sulfato, Het¹oxy, Het²oxy, alkylaminoalkyloxy, cycloalkyloxy, cycloalkylalkyloxy, Het¹alkyloxy, Het²alkyloxy, amino, alkylamino, alkenylamino, alkylcarbonylamino, arylcarbonylamino, formylamino, arylamino, cycloalkylamino, Het¹amino, Het²-amino, alkylsulfonylamino, phosphatyl, thiol, alkylthio, arylthio, alkylsulfonyl, arylsulfonyl, alkenylcarbonyloxy, —O—R⁸—O—R⁹, or saccharyl, wherein
R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, acyl, carboxyamino, acylamino, acetamido, haloalkoxy, alkylcarbonyl, arylcarbonyl, aryl, aralkyl, Het¹, Het², cycloalkyl, carboxyl, oxo, cyano, nitro, amino, aminoalkyl, alkylamino, arylamino, alkylamido, Het¹amino, Het²-amino, or phosphatyl,
and wherein the dotted line represents an optional double bond,
with the proviso that:
when R¹ is hydrogen, R² and R⁴ are hydroxyl, R⁵ is hydrogen or methyl, and R⁶ is an oxo, then R⁷ is not hydroxyl when R³ is hydroxyl, when R¹ is hydrogen, R² and R⁴ are hydroxyl, R⁵ is hydrogen or methyl, R⁶ is an oxo and the dotted line represents a double bond, then R⁷ is not hydroxyl or methoxy when R³ is hydroxyl or methoxy,
when R¹, R², R⁴ are acetoxy, R⁵ is hydrogen, R⁶ is an oxo, and R⁷ is hydroxyl, methoxy, or acetoxy, then R³ is not hydroxyl or acetoxy,
when R¹ is hydrogen, R³ and R⁴ together with the carbon atoms to which they are attached form 2,2-dimethyl-1,3-dioxolane, R⁵ is hydrogen, 4-methoxybenzyl, or methyl, and R⁶ is an oxo, then R⁷ is not hydrogen or hydroxyl when R² is hydroxyl, methoxy, ethoxy, acetoxy or benzyloxy, and R⁷ is not acetoxy when R² is acetoxy, and R⁷ is not methoxy when R² is hydroxyl, methoxy, or acetoxy,
when R², R³, and R⁴ are acetoxy, R⁵ is hydrogen, R⁶ is an oxo, and R¹ is hydrogen, benzoyloxy, or acetoxy, then R⁷ is not hydrogen, hydroxyl, acetoxy, or methoxy,
when R², R³, and R⁴ are hydroxyl, R⁵ is hydrogen or 4-methoxybenzyl, R⁶ is an oxo, and R⁷ is hydrogen, methoxy or hydroxyl, then R¹ is not tetrahydropyranyloxy, benzoyloxy, benzyloxy, or 1-O-3-hydroxybutyryl,
when R¹ is hydrogen, R³ and R⁴ are acetoxy, R⁵ is hydrogen, R⁶ is an oxo, and R⁷ is methoxy or acetoxy, then R² is not benzoyloxy or acetoxy,
when R¹ is hydrogen, R³ and R⁴ are methoxy, R⁵ is methyl, R⁶ is an oxo, and R⁷ is methoxy, then R² is not methoxy,
when R², R³, and R⁴ are acetoxy, R⁵ is hydrogen, R⁶ is an oxo, and R⁷ is acetoxy, then R¹ is not hydrogen,
when R², R³, and R⁴ are hydroxyl, R⁵ is hydrogen, R⁶ is an oxo, and R⁷ is hydroxyl or methoxy, then R¹ is not benzoyloxy,
when R¹ is hydroxyl, R³ and R⁴ together with the carbon atoms to which they are attached form 2,2-dimethyl-1,3-dioxolane, R⁵ is hydrogen or 4-methoxybenzyl, and R⁶ is an oxo, then R⁷ is not hydrogen, hydroxyl, methoxy, or acetoxy when R² is hydroxyl or acetoxy,
when R¹ is hydroxyl or hydrogen, R², R³, and R⁴ are hydroxyl, R⁵ is hydrogen, R⁶ is an oxo, then R⁷ is not hydrogen, hydroxyl, methoxy, or benzyloxy,
when R¹ is hydrogen, R², R³, and R⁴ are benzoyloxy, R⁵ is hydrogen, or benzoyl or benzoyloxy, R⁶ is an oxo, then R⁷ is not hydrogen or methoxy,
when R¹ is hydrogen, R² and R³ and R⁴ are benzoyloxy, R⁶ is an oxo, and R⁷ is methoxy, then R³ is not methoxy,
when R¹, R², R³, and R⁴ are benzoyloxy, R⁵ is hydrogen or benzoyl, and R⁶ is an oxo, then R⁷ is not hydrogen,
when R¹ is acetyloxy, R², R³, and R⁴ are benzoyloxy, R⁵ is hydrogen, R⁶ is an oxo, then R⁷ is not hydrogen,
when R¹, R², R³ and R⁴ are hydroxyl or acetyloxy, R⁵ is hydrogen, R⁶ is oxo, R⁷ is not phosphatyl,
when R¹ is hydrogen, R², R³, and R⁴ are acetoxy, R⁵ is hydrogen, R⁶ is an oxo, then R⁷ is not hydrogen, and
when R⁶ is hydrogen, R⁷ is not hydrogen.

2. The compound according to claim 1, having one of the structural formula 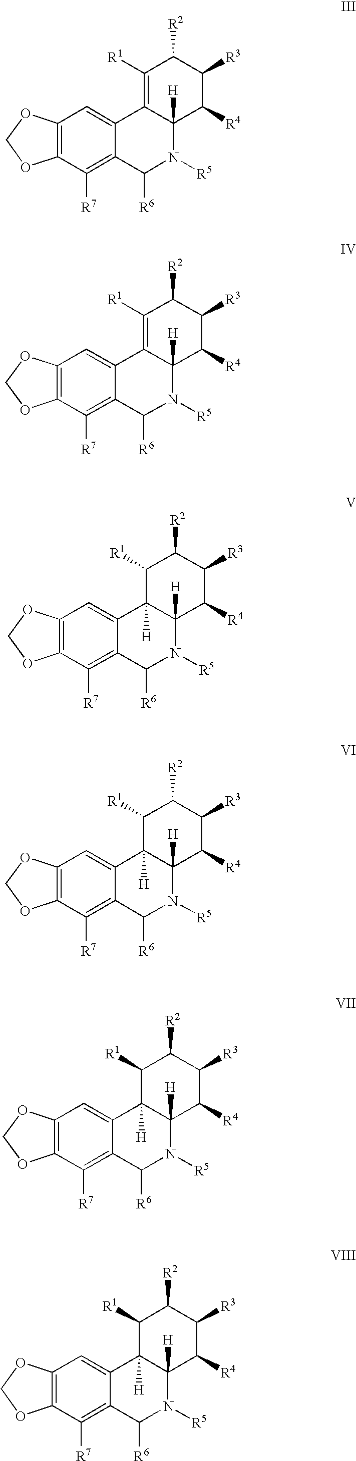
wherein R¹, R², R³, R⁴, R⁵, R⁶, and R⁷ have the same meaning as that defined in claim 1.

3. The compound according to claim 1, wherein
R³ is a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²-amino, Het¹carbonyloxy, Het²-carbonyloxy, cycloalkylcarbonyloxy, alkoxy, aryloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano nitro, aminoalkyl, alkylamino, alkenylamino, arylamino, Het¹amino, Het²amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl,
R⁴ is a group selected from hydroxyl, alkylcarbonyloxy, alkoxy, aryloxy, sulfato, arylalkyloxy, arylcarbonyloxy, amino, alkylamino, arylamino, alkylaminocarbonyloxy, arylaminocarbonyloxy, alkylaminothiocarbonyloxy, arylaminothiocarbonyloxy, Het¹amino, Het²ammo, Het¹carbonyloxy, Het²-carbonyloxy, cycloalkylcarbonyloxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, alkyloxycarbonylalkyl, carboxyalkyl, alkylaminoalkyl, or saccharyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, alkenylamino, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, or hydroxyalkyl, and R¹, R², R⁵, R⁶, and R⁷ have the same meaning as that defined in claim 1.

4. The compound according to claim 1, wherein R³ and R⁴ together with the carbon atoms to which they are attached form a dioxolane ring, said ring being optionally substituted with one or two substituents selected from alky or aryl,
and R¹, R², R⁵, R⁶, and R⁷ have the same meaning as that defined in claim 1.

5. The compound according to claim 1, wherein R¹ is hydrogen or hydroxyl, or a group selected from alkyl, aryl, alkoxy, alkylaminocarbonyloxy, arylaminocarbonyloxy, alkylaminothiocarbonyloxy, arylaminothiocarbonyloxy, arylamino, alkylcarbonyloxy, arylcarbonyloxy, Het¹carbonyloxy, Het²carbonyloxy, cycloalkylcarbonyloxy, aralkyl, aryloxy, cycloalkyl, cycloalkyloxy, Het¹, Het², Het¹alkyl, Het²alkyl, Het¹ oxy, Het²oxy, or saccharyl each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino,
and R², R³, R⁴, R⁵, and R⁶ have the same meaning as that defined in claim 1.

6. The compound according to claim 1, wherein R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkenylcarbonyloxy, Het¹amino, Het²-amino, Het¹carbonyloxy, Het²carbonyloxy, cycloalkylcarbonyloxy, alkoxy, alkenyloxy, alkynyloxy, alkylaminoalkyl, alkylcarbonyl, alkylcarbonylamino, cycloalkyloxy, alkylthio, formylamino, Het¹oxy, Het²oxy, Het¹alkyloxy, Het²alkyloxy, aryloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, glucosaminyl, N-acetyl-glucosaminyl, octylglucopyranosyl, octylribofuranosyl, cyclohexylglucopyranosyl, cyclohexylxylofuranosyl, benzylglucopyranosyl, benzylarabinofuranosyl, N-acetyl-lactosaminyl, acosaminyl, amicetosyl, amylosyl, apiosyl, arcanosyl, ascarylosyl, bacillosaminyl, boivinosyl, cellotriosyl, chacotriosyl, chalcosyl, cladinosyl, colitosyl, cymarosyl, daunosaminyl, desosaminyl, d-glycero-L-gulo-heptosyl, diginosyl, digitalosyl, digitoxosyl, evalosyl, evernitrosyl, forosaminyl, fucosaminyl, garosaminyl, hamamelosyl, isolevoglucosenonyl, kanosaminyl, kansosaminyl, lactosaminyl, lactosediaminyl, fucitolyl, maltulosyl, mannosaminyl, melezitosyl, mycaminosyl, mycarosyl, mycinosyl, mycosaminyl, noviosyl, oleandrosyl, paratosyl, perosaminyl, planteosyl, pneumosaminyl, purpurosaminyl, quinovosaminyl, quinovosyl, rhamnitolyl, rhamnosaminyl, rhodinosyl, rhodosaminyl, sarmentosyl, solatriosyl, stachyosyl, streptosyl, umbelliferosyl, trehalosaminyl, 1,6-anhydro-D-glucopyranosyl, 1-hydroxy-α-D-allopyranosyl, 2,3:5,6-di-O-isopropylidene-D-mannofuranosyl, 2-amino-2-deoxy-D-galactitolyl, 2-deoxy ribosyl, 2-deoxyglucosyl, 5-amino-5-deoxy-D-glucopyranosyl, 6-deoxy-D-galactitolyl, 2-amino-2-deoxy glucosyl, 2-acetamido-2-deoxy-glucosyl, 2-amino-2-deoxy galactosyl, 2-acetamido-2-deoxy-galactosyl, 2-amino-2-deoxy mannosyl, 2-acetamido-2-deoxy-mannosyl, 2-acetamido-2-deoxy-4-O-β-D-galactosyl-D-glucosyl, 2-amino-2-deoxy-4-O-β-D-galactosyl-D-glucosyl, 6′-N-acetylglucosaminyllactosyl, 2-acetamido-2-deoxy-3-O-α-L-fucosyl-D-glucosyl, 6-O(2-acetamido-2-deoxy-β-D-glucosyl)-D-galactosyl, 2-acetamido-2-deoxy-3-O-β-O-D-galactosyl-D-glucosyl, 2′-acetamido-2′-deoxy-3-O-β-D-glucosyl-D-galactosyl, 3-fucosyl-D-lactosyl, or 3-fucosyl-2-acetamido-2-deoxy-4-O-β-D-galactosyl-D-glucosyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thio, alkyl, haloalkyl, alkoxy, acyl, carboxyl, carboxyamino, acylamino, acetamido, haloalkoxy, alkylcarbonyl, aryl, aralkyl, Het¹, Het², cycloalkyl, carboxyl, oxo, cyano nitro, amino, aminoalkyl, alkylamino, alkenylamino, alkylamido, arylamino, Het¹amino, Het²-amino, alkylaminosulfonyl, alkylcarbonylamino, alkyloxyaminoalkenyl, alkyloxycarbonyl, alkylsulfonyl, alkylsulfonylamino, alkylthio, arylcarbonyl, arylcarbonylamino, aryloxy, haloaryl, Het¹alkyl, Het²alkyl, hydroxyalkyl, or phosphatyl,
and R¹, R³, R⁴, R⁵, and R⁶ have the same meaning as that defined in claim 1.

7. The compound according to claim 1, wherein R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkenylcarbonyloxy, Het¹amino, Het²-amino, Het¹carbonyloxy, Het²-carbonyloxy, cycloalkylcarbonyloxy, alkoxy, cycloalkyloxy, Het¹oxy, Het²oxy, Het¹alkyloxy, Het²alkyloxy, aryloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemarmosyl, or glucosaminyl, L or D isomers thereof, α or β form thereof, pyranuronic or furanuronic form thereof, pyranose or furanose form thereof, combination thereof, deoxy derivatives thereof; hydroxyl-protected acetate or benzoyl derivatives thereof, amino derivatives thereof; amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, arylcarbonyl, aryl, aralkyl, Het¹, Het², cycloalkyl, carboxyl, acyl, carboxyamino, acylamino, acetamido, alkylamido, thiol, oxo, cyano, phosphatyl, nitro, or amino,
and R¹, R³, R⁴, R⁵, and R⁶ have the same meaning as that defined in claim 1.

8. The compound according to claim 1, wherein R⁵ is hydrogen or a group selected from alkyl, alkyloxycarbonylalkyl, aryloxycarbonylalkyl, aryl, aralkyl, alkenyl, alkynyl, alkylaminoalkyl, alkylcarbonyl, arylcarbonyl, cycloalkyl, Het¹alkyl, Het²alkyl, alkyloxycarbonyl, aminocarbonyl, halo alkylcarbonylaminocarbonyl, alkylcarbonylaminocarbonyl, or cycloalkylalkyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, or amino,
and R¹, R², R³, R⁴, R⁷, and R⁶ have the same meaning as that defined in claim 1.

9. The compound according to claim 1, wherein R⁷ is hydrogen, hydroxyl, or a group selected from alkoxy, alkenyloxy, alkynyloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, alkylaminocarbonyloxy, arylaminocarbonyloxy, alkylaminothiocarbonyloxy, arylaminothiocarbonyloxy, hydroxycarbonylalkyloxy, Het¹carbonyloxy, Het²-carbonyloxy, aryloxy, sulfato, Het¹oxy, Het²oxy, alkylaminoalkyloxy, cycloalkyloxy, cycloalkylalkyloxy, Het¹alkyloxy, Het²alkyloxy, amino, alkylamino, alkenylamino, alkylcarbonylamino, arylcarbonylamino, formylamino, arylamino, Het¹amino, Het²-amino, alkylsulfonylamino, cycloalkylamino, sulfato, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof; amido derivatives thereof, thio derivatives thereof, di-, tri-, oligo-, or polysaccharides thereof, wherein is R⁸ is alkylene or alkyl, and R⁹ is selected from hydrogen, alkyl, aryl, Het¹, Het², or cycloalkyl; each group being optionally substituted with one or more substituents independently selected from halogen, thiol, alkyl, haloalkyl, alkoxy, acyl, carboxyamino, acylamino, acetamido, haloalkoxy, alkylcarbonyl, arylcarbonyl, aryl, aralkyl, Het¹, Het², cycloalkyl, carboxyl, oxo, cyano, phosphatyl, nitro, amino, aminoalkyl, alkylamino, arylamino, alkylamido, Het¹amino, or Het²-amino,
and R¹, R², R³, R⁴, R⁵, and R⁶ have the same meaning as that defined in claim 1.

10. The compound according to claim 1, wherein R⁷ is hydrogen, hydroxyl, or a group selected from alkoxy, alkenyloxy, alkynyloxy, aryloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, alkylaminocarbonyloxy, arylaminocarbonyloxy, sulfato, alkylaminothiocarbonyloxy, arylaminothiocarbonyloxy, Het¹carbonyloxy, Het²-carbonyloxy, Het¹oxy, Het²oxy, cycloalkyloxy, cycloalkylalkyloxy, Het¹alkyloxy, Het²alkyloxy, amino, alkylamino, alkenylamino, alkylcarbonylamino, arylcarbonylamino, formylamino, arylamino, Het¹amino, Het²amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, mannoheptulosyl, sedoheptulosyl, abequosyl, isomaltosyl, kojibiosyl, laminaribiosyl, nigerosyl, primeverosyl, rutinosyl, tyvelosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, maltopentaosyl, maltohexaosyl, maltoheptaosyl, sicosyl, panosyl, isopanosyl, inosyl, N-acetylgalactosaminyl, mannotriosyl, globotriosyl, erlosyl, neotrehalosyl, chitobiosyl, chitobiosemannosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, Het¹, Het², cycloalkyl, carboxyl, oxo, cyano, nitro, phosphatyl, amino, aminoalkyl, alkylamino, arylamino, acyl, carboxyamino, acylamino, acetamido, alkylamido, Het¹amino, or Het²-amino,
and R¹, R², R³, R⁴, R⁵, and R⁶ have the same meaning as that defined in claim 1.

11. A compound according to claim 1, having one of the structural formula 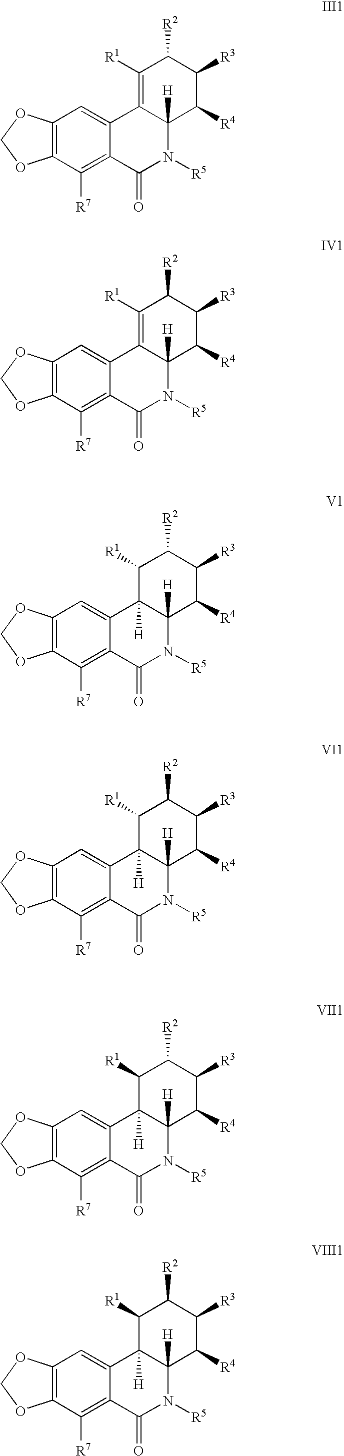 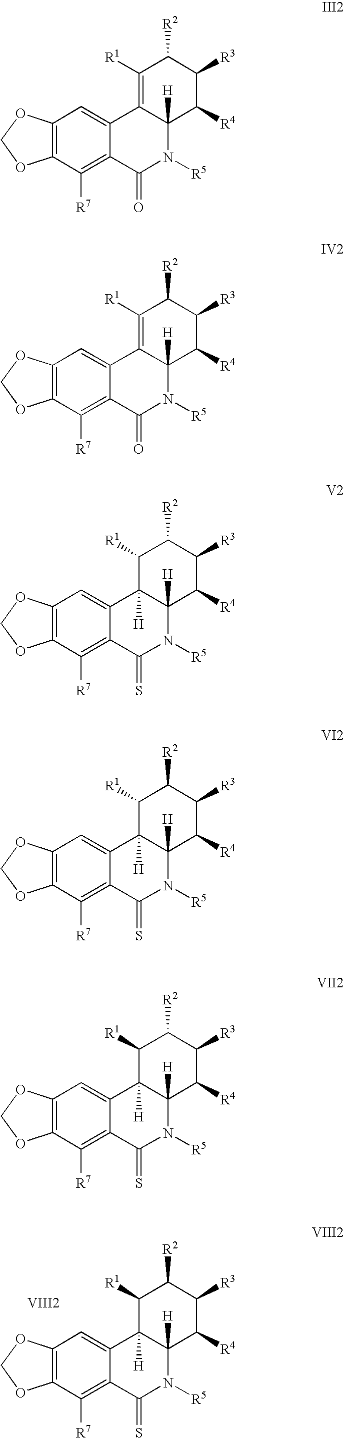 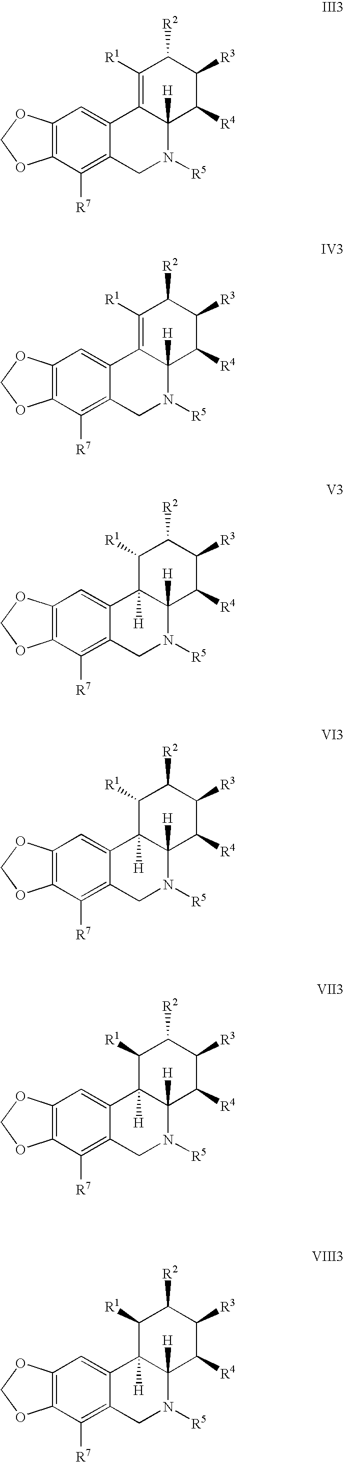
wherein R¹, R², R³, R⁴, R⁵, and R⁷ have the same meaning as that defined in claim 1.

12. The compound according to claim 11, wherein
R¹ is hydrogen, hydroxyl, or a group selected from alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, Het¹carbonyloxy, Het²carbonyloxy, cycloalkylcarbonyloxy, aralkyl, cycloalkyl, arylamino, Het¹, Het², glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino,
R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkoxy, aryloxy, alkenylcarbonyloxy, Het¹amino, Het²-amino, Het¹carbonyloxy, Het²-carbonyloxy, cycloalkylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, Het¹, Het², cycloalkyl, carboxyl, oxo, cyano, phosphatyl, nitro, or amino,
R³ is a group selected from hydroxyl, alkylcarbonyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkoxy, aryloxy, alkenyloxy, alkynyloxy, cycloalkyloxy, Het¹oxy, Het²oxy, Het¹alkyloxy, Het²alkyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof; di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, or nitro,
R⁴ is a group selected from hydroxyl, alkylcarbonyloxy, alkoxy, aryloxy, sulfato, arylalkyloxy, arylcarbonyloxy, alkylaminocarbonyloxy, arylaminocarbonyloxy, alkylaminothiocarbonyloxy, arylaminothiocarbonyloxy, Het¹carbonyloxy, Het²-carbonyloxy, cycloalkylcarbonyloxy, alkenyloxy, alkynyloxy, cycloalkyloxy, Het¹oxy, Het²oxy, Het¹alkyloxy, Het²alkyloxy, alkyloxycarbonylalkyl, carboxyalkyl, alkylaminoalkyl, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino,
R⁵ is hydrogen or a group selected from alkyl, alkyloxycarbonylalkyl, aryloxycarbonylalkyl, aryl, aralkyl, alkenyl, alkynyl, cycloalkyl, Het¹alkyl, Het²alkyl, alkylcarbonyl, arylcarbonyl, alkyloxycarbonyl, aminocarbonyl, halo alkylcarbonylaminocarbonyl, alkylcarbonylaminocarbonyl, or cycloalkylalkyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, or amino, and
R⁷ is hydrogen, hydroxyl or a group selected from alkoxy, alkenyloxy, alkynyloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, alkylaminocarbonyloxy, arylaminocarbonyloxy, alkylaminothiocarbonyloxy, arylaminothiocarbonyloxy, Het¹carbonyloxy, Het²-carbonyloxy, aryloxy, Het¹oxy, Het²oxy, cycloalkyloxy, cycloalkylalkyloxy, Het¹alkyloxy, Het²alkyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, Het¹, Het², cycloalkyl, carboxyl, acyl, carboxyamino, acylamino, acetamido, acetamido, oxo, cyano, phosphatyl, nitro, or amino.

13. The compound according to claim 11, wherein
R¹ is hydrogen, hydroxyl, or a group selected from alkyl, aryl, alkoxy, aryloxy, alkylaminocarbonyloxy, arylaminocarbonyloxy, alkylaminothiocarbonyloxy, arylaminothiocarbonyloxy, alkylcarbonyloxy, arylcarbonyloxy, Het¹carbonyloxy, Het²-carbonyloxy, cycloalkylcarbonyloxy, aralkyl, cycloalkyl, arylamino, Het¹, Het², glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, amino, aminoalkyl, alkylamino, arylamino, Het¹amino, or Het²-amino,
R² is oxo or a group selected from hydroxyl, alkylcarbonyloxy, arylcarbonyloxy, amino, alkylamino, arylamino, alkylaminocarbonyloxy, arylaminocarbonyloxy, alkylaminothiocarbonyloxy, arylaminothiocarbonyloxy, alkoxy, aryloxy, alkenylcarbonyloxy, Het¹amino, Het²-amino, Het¹carbonyloxy, Het²-carbonyloxy, cycloalkylcarbonyloxy, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, each group being optionally substituted with one or more substituents independently selected from halogen, thiol, alkyl, haloalkyl, alkoxy, haloalkoxy, alkylcarbonyl, arylcarbonyl, aryl, aralkyl, acyl, carboxyamino, acylamino, acetamido, alkylamido, Het¹, Het², cycloalkyl, carboxyl, oxo, cyano nitro, or amino,
R³ and R⁴ together with the carbon atoms to which they are attached form a dioxolane ring, said ring being optionally substituted with one or two substituents selected from alkyl or aryl,
R⁵ is hydrogen or a group selected from alkyl, alkyloxycarbonylalkyl, aryloxycarbonylalkyl, aryl, aralkyl, alkenyl, alkynyl, cycloalkyl, Het¹alkyl, Het²alkyl, cycloalkylalkyl, alkylcarbonyl, alkyloxycarbonyl, aminocarbonyl, halo alkylcarbonylaminocarbonyl, alkylcarbonylaminocarbonyl, or arylcarbonyl, each group being optionally substituted with one or more substituents independently selected from halogen, alkyl, haloalkyl, alkoxy, haloalkoxy, alkylcarbonyl, aryl, aralkyl, Het¹, Het², cycloalkyl, carboxyl, oxo, cyano, nitro, or amino, and
R⁷ is hydrogen, hydroxyl, or a group selected from alkoxy, alkenyloxy, alkynyloxy, aralkyloxy, alkylcarbonyloxy, arylcarbonyloxy, arylcarbonylalkyloxy, carboxyalkyloxy, alkyloxycarbonylalkyloxy, aryloxycarbonylalkyloxy, hydroxycarbonylalkyloxy, alkylaminocarbonyloxy, arylaminocarbonyloxy, alkylaminothiocarbonyloxy, arylaminothiocarbonyloxy, Het¹carbonyloxy, Het²-carbonyloxy, aryloxy, Het¹oxy, Het²oxy, cycloalkyloxy, cycloalkylalkyloxy, Het¹alkyloxy, Het²alkyloxy, amino, alkylamino, alkenylamino, alkylcarbonylamino, arylcarbonylamino, formylamino, arylamino, Het¹amino, Het²-amino, alkylsulfonylamino, cycloalkylamino, phosphatyl, thiol, alkylthio, arylthio, alkylsulfonyl, arylsulfonyl, alkenylcarbonyloxy, —O—R⁸—O—R⁹, glucosyl, fructosyl, galactosyl, mannosyl, ribosyl, ribulosyl, xylulosyl, erythrosyl, erythrulosyl, rhamnosyl, threosyl, sorbosyl, psicosyl, tagatosyl, fucosyl, arabinosyl, xylofuranosyl, lyxosyl, talosyl, psicosyl, idosyl, gulosyl, altrosyl, allosyl, isomaltosyl, maltosyl, lactosyl, sucrosyl, cellobiosyl, trehalosyl, gentiobiosyl, melibiosyl, turanosyl, sophorosyl, isosucrosyl, raffinosyl, palatinosyl, lactulosyl, gentianosyl, 3-mannobiosyl, 6-mannobiosyl, 3-galactobiosyl, 4-galactobiosyl, maltotriosyl, maltotetraosyl, isomaltotriosyl, inosyl, mannotriosyl, globotriosyl, erlosyl, neotrehalosyl, or glucosaminyl, L or D isomers thereof, α or β form thereof, pyranuronic or furanuronic form thereof, pyranose or furanose form thereof, combination thereof, deoxy derivatives thereof, hydroxyl-protected acetate or benzoyl derivatives thereof, amino derivatives thereof, amido derivatives thereof, thio derivatives thereof, di-, tri-, oligo-, or polysaccharides thereof, wherein is R⁸ is alkylene or alkenylene and R⁹ is selected from hydrogen, alkyl, aryl, Het¹, Het², or cycloalkyl; each group being optionally substituted with one or more substituents independently selected from halogen, thiol, alkyl, haloalkyl, alkoxy, haloalkoxy, alkylcarbonyl, arylcarbonyl, aryl, aralkyl, Het¹, Het², cycloalkyl, carboxyl, acyl, carboxyamino, acylamino, acetamido, acetamido, oxo, cyano, nitro, or amino.

14. (canceled)

15. A pharmaceutical composition comprising a pharmaceutically acceptable excipient and a therapeutically effective amount of at least one compound according to claim 1.

16. A method of treating cancer comprising administering a pharmaceutical composition according to claim 15 to a patient in need thereof.

17. A method for the prevention and/or treatment of cancer, and/or for preventing, treating, and/or alleviating complications, and/or symptoms, and/or inflammatory responses associated therewith comprising administering a compound according to claim 1 to a patient in need thereof.

18. The method according to claim 17, wherein said cancer is selected from leukemia, non-small cell lung cancer, small cell lung cancer, CNS cancer, melanoma, ovarian cancer, kidney cancer, prostate cancer, breast cancer, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, or lymphoma.

19. The method according to claim 16, wherein said cancer is selected from non-small cell lung cancer, CNS cancer, melanoma, ovarian cancer, kidney cancer, prostate cancer, breast cancer, colon cancer, bladder cancer, sarcoma, pancreatic cancer, colorectal cancer, head and neck cancer, liver cancer, stomach cancer, oesophageal cancer, or lymphoma.

20. A method for treating and/or preventing cancer comprising administrating to an individual in need of such a treatment a therapeutically effective amount of at least one compound according to claim 1.

21. The method of claim 20, wherein the cancer is treated.

22. The method according to claim 20, wherein said cancer is selected from leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, or lymphoma.

23. The method according to claim 22, wherein said cancer is selected from non-small cell lung cancer, CNS cancer, melanoma, ovarian cancer, kidney cancer, prostate cancer, breast cancer, colon cancer, bladder cancer, sarcoma, pancreatic cancer, colorectal cancer, head and neck cancer, liver cancer, stomach cancer, oesophageal cancer, or lymphoma.
